SEQUENCE LISTING (2 sequences)

SEQ ID NO: 1 - protein, 643 aa, Artificial Sequence
  MPK38 amino acid sequence
MKDYDELLKYYELYETIGTGGFAKVKLACHVLTGEMVAIKIMDKNALGSDLPRVKTEIDALKSLRHQHICQLYHVLETKNKIFMVLEYCPGGELFDYIISQDRLSEEETRVVFRQILSAVAYVHSQGYAHRDLKPENLLFDENHKLKLIDFGLCAKPKGNKDYHLQTCCGSLAYAAPELIQGKSYLGSEADVWSMGILLYVLMCGFLPFDDDNVMALYKKIMRGKYEVPKWLSPSSILLLQQMLQVDPKKRISMRNLLNHPWVMQDYSCPVEWQSKTPLTHLDEDCVTELSVHHRSSRQTMEDLISSWQYDHLTATYLLLLAKKARGKPARLQLLSFSCGTASTTPKSKNLSLEDMSTSDDNCVAGLIDYELCEDKLLAPKTPQVTKHLAESNHAASKSPAPGVRRAVANKLMDKENVCTPKSSVKNEEQFVFSEPKIPVSKNQYKREIPASPTRFPTPAKARAQCLREAPVRTPGNSAGADTLTTGVISPERRCRSMDVDLNQAHMEDTPKKKGTNVFGSLERGLDKVLTALTRNKKKGSARDGPRKRKLHYNVTTTRLVNPDQLLSEIMAILPKKNVDFVQKGYTLKCQTQSDFGKVTMQFELEVCQLQRPDVVGIRRQRLKGDAWVYKRLVEDILSGCKM

SEQ ID NO: 2 - DNA, 2581 bp, Artificial Sequence
  MPK38 amino acid sequence
aagaggtaatccatctatgtcgggtggtcgacaagaggtaatccctttggcgggtggaagctgctggaacttgaatcctgcagaaattcccaccgacttgttccggctttgctagactctgcagccggctgcttcaccttctgtcctgttcggtcctgttcggtccttgctgggcccgctgcccttcaggttctggttgtcattttgaataaccttccaggtggactatgaaagattatgacgaactccttaaatactatgaactatatgaaacgattgggacaggtggctttgcaaaggtcaaactggcctgccatgtcctcactggagagatggtagctataaaaatcatggataagaatgcgctagggagtgatttgccccgagtcaaaactgagatcgatgcgctgaagagtctgagacatcagcacatatgtcagctctaccatgtgctggagacaaagaacaaaatattcatggttctggagtactgtccaggaggagagctgtttgactacataatctcccaggatcgcctgtcggaagaggagacccgggtcgtcttccgtcagatactgtctgcagttgcgtatgtccacagccagggctatgcccacagggacctcaaaccagaaaatttattatttgatgaaaatcataagctaaagctgattgactttggtctttgtgcaaaacccaagggcaacaaggactaccatctgcagacgtgctgtgggagccttgcttatgcagctcctgaactaatacaagggaagtcgtaccttggatcagaggcagatgtttggagcatgggcatcctcctgtatgtgctcatgtgtggatttctaccatttgatgatgataatgtcatggctttgtacaagaagataatgagagggaaatacgaagttcctaagtggctctctcccagtagcattctgcttctccagcagatgttgcaggtggacccaaagaaacggatttctatgagaaatctcctgaaccatccctgggtcatgcaagattacagctgtcccgtggagtggcaaagcaagactcctttgactcacctcgatgaggattgcgtgacagagctttctgtacatcaccgcagcagcaggcagacaatggaggatttaatttcgtcgtggcggtacgatcacctcacagccacctaccttctgcttctagccaagaaggcccgggggaagccggctcgtctacagctcctgtccttctcttgtggaaccgccagcaccactccaaagtcaaagaatctgagcctggaagatatgagcacaagtgatgataactgtgtggctggattgatagactatgaattgtgtgaagataaattattagctcccaagacgccacaggttaccaaacacttggcagaatcaaatcacgcagcatctaaatcaccagcgccaggggtacgcagagcagtggcaaataaattaatggacaaagaaaatgtgtgcactcccaagtcttctgtgaagaatgaagagcagtttgtattttctgagccgaagattccagttagtaagaaccagtataagagagaaatacccgcctcaccaacccgtttcccaacacctgcaaaagctagagcccagtgcctgagagaagccccggttagaacaccagggaattccgcaggagcagacacactaacgacaggtgtcattagccccgagaggaggtgccgttcaatggacgtggatctcaaccaggcacacatggaggataccccgaaaaagaaaggaaccaatgtgtttgggagccttgagagaggactggataaggttctcactgcgctcacaaggaacaagaagaagggctctgccagagatggaccaagaaagcgaaagctgcactacaatgtgactacaactcgcctggtgaaccccgaccagctcctgagcgaaatcatggctattcttccaaagaagaacgtggacttcgtacagaaaggttacactctaaagtgtcaaacgcagtctgattttggcaaagtgacaatgcagtttgaactggaagtgtgccagctgcagagacctgacgtggtaggcatccggagacagcggctgaagggtgatgcctgggtttacaagagattagtggaagatatcttgtctggctgcaagatgtgaccgatgacagtgtccgtctgcccggatgaatgtgggtgtgatgttgcacacaggaagactgtgagattggttccatttgggggccattggatctatggaactacaagctcatttaaatagccatcttcaagaccaatgtatttttgctttttaaacaaaaatttttactttgtggctgaatccaaggcaactgttctgtcattatcaattactctctgttttaatcatgggcttggtatgttagtaactgtttttatattcacttccatcaggggatgccagctcttcactgtgactcattttgatgtacagtttctttctgaactaaaaccatttgtgaatatatcaagctctttttttgtatctgattttgatccaaataaaacctcgaatggcttcctgactgttaaaaaaaaaaaaaa

(19) United States
(12) Patent Application Publication
(10) Pub. No.: US 2022/0241382 A1
(43) Pub. Date: Aug. 4, 2022

(54) NOVEL USE OF MPK38/MELK AS THERAPEUTIC AGENT FOR OBESITY OR METABOLIC DISEASES SPECIFIC TO MIDDLE-AGED MEN

(71) Applicant: Chungbuk National University Industry-Academic Cooperation Foundation, Chungcheongbuk-do (KR)

(72) Inventors: Hyunjung HA, Chungcheongbuk-do (KR); Hyun-A SEONG, Chungcheongbuk-do (KR)

(21) Appl. No.: 17/346,057

(22) Filed: Jun. 11, 2021

(30) Foreign Application Priority Data
Feb. 3, 2021 (KR) 10-2021-0015637

(51) Int. Cl. A61K 38/45; A61P 3/04; A61P 3/08

(52) U.S. Cl. CPC A61K 38/45; A61P 3/08; A61P 3/04

(57) ABSTRACT

Disclosed is a novel use of MPK38/MELK as a therapeutic agent for obesity or metabolic diseases specific to middle-aged men. Provided are a pharmaceutical composition and health functional food for preventing or treating obesity or a metabolic disease specific to middle-aged men, containing a murine protein serine-threonine kinase 38 (MPK38) protein or a gene encoding the same as an active ingredient. Obesity and metabolic disorders were found to occur in MPK38-knockout middle-aged male mice. As a result of inducing the expression of MPK38 in MPK38-knockout middle-aged male mice, MPK38 was found to have effects of decreasing the size of adipocytes, suppressing the expression of adipogenic genes, reducing glucose and insulin in the blood, enhancing insulin sensitivity, reducing triglycerides and total cholesterol in the blood, promoting ketone formation, and promoting the production of testosterone, a male hormone, in middle-aged male mice. In particular, it was found that the effects of the MPK38 are specific to middle-aged men and that MPK38 is useful as a therapeutic agent and health functional food for treating obesity or metabolic diseases specific to middle-aged men.

### BACKGROUND OF THE INVENTION

### Field of the Invention

The present invention relates to a novel use of MPK38/MELK as a therapeutic agent for obesity or metabolic diseases specific to middle-aged men.

### Description of the Related Art

Both men and women experience aging-related physical and mental decline starting at middle age. Like women, men start to undergo menopause after their 40s, and the likelihood of onset thereof increases with age, and adrenal secretion function, sperm fertility, the function of Leydig cells, and serum testosterone decrease, accompanied by obesity, various metabolic diseases, and andropause symptoms. Middle-aged men show aging-related symptoms of decreased muscle mass and strength, decreased body mass index, decreased body hair, skin changes, deteriorated intellectual capability and spatial perception, fatigue, decreased sexual desire and erectile dysfunction, and mood swings, accompanied by depression and mental instability, as well as complications such as decreased bone density and increased visceral fat.

In particular, in the case of middle-aged obesity, there is a difference in the cause of onset between men and women. One of the main causes of male middle-aged obesity and metabolic diseases is a decrease in male hormones. The male hormone testosterone is the main circulating androgen in men, is mainly secreted by the Leydig cells in the testis, and is mainly involved in the development and maintenance of male secondary sexual characteristics.

Therefore, male hormone therapy is used as a method for treating male obesity and metabolic diseases, but has a problem of occurrence of unexpected side effects, and is limited in that it is not a treatment of the fundamental causes thereof.

Therefore, it is necessary to identify more fundamental causes and mechanisms of action with regard to obesity and metabolic diseases specific to middle-aged men and thus to develop a novel therapeutic agent capable of effectively preventing or treating the obesity and metabolic diseases.

Meanwhile, murine protein serine-threonine kinase 38 (MPK38) (also known as maternal embryonic leucine zipper kinase (MELK) is an AMP-activated protein kinase (AMPK) that is highly phylogenetically conserved, and is known to have a tumor-promoting function due to anti-apoptotic activity and to be involved in metabolic functions due to the pro-apoptotic action thereof. However, the effects of MPK38/MELK on obesity and metabolic diseases specific to middle-aged men have not been studied yet.

Accordingly, the present inventors found that abnormalities in obesity, sugar, lipid and energy metabolism occur in MPK38/MELK-deficient middle-aged male mice, and in particular that these symptoms occur specifically in middle-aged males, and injection of MPK38/MELK and induction of overexpression using adenovirus alleviate obesity symptoms in middle-aged male mice and affect the production of testosterone hormone. As a result, the present inventors identified that MPK38/MELK can be used as a therapeutic agent for preventing, ameliorating or treating obesity and metabolic diseases specific to middle-aged men. Based on this finding, the present invention has been completed.

### PRIOR ART

(Patent Document 1) Korean Patent No. 10-2170090

(Patent Document 2) Korean Patent No. 10-1682083

### SUMMARY OF THE INVENTION

Accordingly, it is one object of the present invention to provide a pharmaceutical composition for preventing or treating obesity or metabolic diseases specific to middle-aged men, containing an MPK38 (murine protein serine-threonine kinase 38)/MELK (maternal embryonic leucine zipper kinase) protein or a gene encoding the same as an active ingredient.

It is another object of the present invention to provide a health functional food for preventing or ameliorating obesity or metabolic diseases specific to middle-aged men containing an MPK38/MELK protein or a gene encoding the same as an active ingredient.

It is another object of the present invention to provide a composition for promoting testosterone production containing an MPK38/MELK protein or a gene encoding the same as an active ingredient.

In accordance with the present invention, the above and other objects can be accomplished by the provision of a pharmaceutical composition for preventing or treating obesity or a metabolic disease specific to middle-aged men, containing an MPK38/MELK protein or a gene encoding the same as an active ingredient.

The protein may have an amino acid sequence of SEQ ID NO: 1.

The gene may have a nucleotide sequence of SEQ ID NO: 2.

The MPK38 gene having the nucleotide sequence of SEQ ID NO: 2 may be inserted into an expression vector.

The MPK38 gene may be specific to middle-aged men and may reduce a size of adipocytes, inhibits expression of C/EBPα (CCAAT-enhancer-binding protein α) and PPARγ (peroxisome proliferator-activated receptor gamma and FABP4) as adipogenic genes, may improve insulin sensitivity, may reduce blood glucose and insulin, may reduce blood triglyceride, total cholesterol, HDL-C and LDL-C levels, and may increase ketone body formation.

The MPK38 may enhance testosterone production.

The metabolic disease may be selected from the group consisting of diabetes, hyperlipidemia, arteriosclerosis, high blood pressure, cardiovascular diseases, fatty liver, obesity-derived inflammatory diseases, obesity-derived autoimmune diseases, and obesity-derived cancer.

In accordance with another aspect of the present invention, provided is a health functional food for preventing or ameliorating obesity or a metabolic disease specific to middle-aged men, containing an MPK38 protein or a gene encoding the same as an active ingredient.

In accordance with another aspect of the present invention, provided is a composition for promoting testosterone production containing an MPK38 protein or a gene encoding the same as an active ingredient.

### BRIEF DESCRIPTION OF THE DRAWINGS

The above and other objects, features and other advantages of the present invention will be more clearly understood from the following detailed description taken in conjunction with the accompanying drawings, in which:

FIGS. 1A-1L show the results of observation of the occurrence of obesity, glucose and lipid metabolism abnormalities, and inflammation in MPK38-knockout middle-aged male mice, wherein FIG. 1A shows the appearance, magnetic resonance images and respective organ morphologies regarding 7-month-old MPK38^{+/+}, MPK38^{+/-} and MPK38^{-/-} male mice; FIG. 1B shows the adipose tissue volume of each mouse experimental group; FIG. 1C shows the body weight (g) and food intake (%) of each mouse experimental group; FIG. 1D shows the glucose and insulin resistance of each mouse experimental group; FIG. 1E shows the blood glucose and insulin concentration under feeding and fasting conditions; FIG. 1F shows 14C-2-deoxy-glucose uptake; FIG. 1G shows the mRNA expression level in the liver of the gluconeogenic gene and the blood glucose concentration; FIG. 1H shows free fatty acid, triglycerides, total cholesterol, HDL-C and LDL-C levels; FIG. 1I shows the lipogenic capacity of adipocytes and the mRNA expression levels of lipogenic genes in white adipose tissue (WAT) and in the liver; FIG. 1J is a graph showing the size distribution of adipocytes and an image showing hematoxylin-eosin-stained epididymal WAT tissue; FIG. 1K shows the concentrations of proinflammatory cytokines; and FIG. 1L shows macrophages in the adipose stromal vascular fraction (SVF) of MPK38^{+/+} and MPK38^{-/-} male mice, detected using flow cytometry, wherein the counts of cells (%) in M1 and M2 are shown;

FIGS. 2A-2D show a process for producing MPK38-knockout mice and abnormalities in lipid metabolisms thereof, wherein FIG. 2A is a schematic diagram showing a process for producing MPK38^{-/-} mice using a gene trap embryonic stem (ES) cell line AR081 (Mutant Mouse Regional Resource Centers of UC Davis, USA); FIG. 2B is an image showing the livers of 7-month-old normal-diet MPK38^{+/+}, MPK38^{+/-} and MPK38^{-/-} male mouse groups stained with hematoxylin-eosin; FIG. 2C shows the activity of AST and ALT; and FIG. 2D shows the mRNA expression levels of adipogenic regulators in the epididymal WAT;

FIGS. 3A-3E show energy consumption of MPK38^{+/+} and MPK38^{-/-} male mice raised under light and dark conditions, wherein FIG. 3A shows body weight and energy consumption; FIG. 3B shows an oxygen consumption profile; FIG. 3C shows a carbon dioxide generation profile; FIG. 3D shows an RER profile; and FIG. 3E shows the mRNA expression levels of thermogenic regulators in BAT;

FIGS. 4A-4Q show the exacerbation of diet-induced metabolic disorders due to MPK38 deficiency in middle-aged male mice, wherein FIG. 4A shows the appearance, weight and respective organ sizes regarding 7-month-old MPK38^{+/+}, MPK38^{+/-}, and MPK38^{-/-} male mice; FIG. 4B shows free fatty acid, triglycerides, AST, ALT, glucose, insulin, leptin and adiponectin in serum; FIG. 4C shows hematoxylin-eosin-stained liver and epididymal WAT tissues (left) and liver triglycerides (right); FIG. 4D shows the mRNA expression levels of the adipogenic regulators in the epididymal WAT; FIG. 4E shows blood glucose and insulin concentrations under feeding and fasting conditions; FIG. 4F shows glucose and insulin resistance; FIG. 4G shows the level of 14C-2-deoxy-glucose uptake in the epididymal WAT and muscle and the expression levels of IRS-PI3K-signaling-pathway-related factors, detected by immunoblotting after injection of insulin into the inferior vena cava; FIG. 4H shows the mRNA expression levels of gluconeogenic genes in the liver; FIG. 4I shows the mRNA expression levels of the lipogenic genes in the epididymal WAT (top) and the lipogenic capacity in adipocytes (bottom); FIG. 4J shows the mRNA expression level of the oxidation gene of fatty acid in the epididymal WAT (top) and the lipolysis of adipocytes by isoproterenol-stimulation (bottom); FIG. 4K shows β-oxidation of the liver, measured using 14C-labeled palmitate; FIG. 4L shows total cholesterol, HDL-C and LDL-C concentrations; FIG. 4M shows the total ketone body in the blood under feeding and fasting conditions; FIG. 4N shows the phosphorylation level of S6 (Ser240/244) (left) in liver lysates derived from the freely fed group, the fasted group, and the high-fat-diet-fed group, and activation of the mTORC1 signaling pathway (right); FIG. 4O shows the mRNA expression levels of ketogenic genes (PPARα, CPT1 and 3-hydroxy-3-methylglutaryl-CoA synthase 2 (HMGCS2)) in the liver; FIG. 4P shows the concentrations of proinflammatory cytokines; and FIG. 4Q shows the macrophages in the adipose stromal vascular fraction (SVF) derived from 7-month-old MPK38^{+/+} and MPK38^{-/-} male mice, detected by flow cytometry, and the counts of cells (%) in M1 and M2, wherein the M1 surface marker is F4/80⁺CD11c⁺CD206⁻ and the M2 surface marker is F4/80⁺CD11c⁺CD206⁺;

FIGS. 5A-50 show that MPK38 has no effect on diet-induced metabolic disorders in high-fat-diet-fed female mice, wherein FIG. 5A is a graph showing adipocyte cell size distribution of high-fat-diet-fed MPK38^{+/+}, MPK38^{+/-} and MPK38^{-/-} female mice (left) and an image showing hematoxylin-eosin-stained epididymal WAT tissue (right); FIG. 5B shows the mRNA levels of adipogenic regulators in the epididymal WAT; FIGS. 5C and 5D show blood glucose and insulin concentrations under feeding and fasting conditions and glucose and insulin resistance, respectively; FIG. 5E shows the degree of 14C-2-deoxy-glucose uptake in vitro upon treatment or non-treatment with human insulin (100 nM) (left) and the expression levels of IRS-PI3K-signaling-related factors upon treatment with insulin, detected by immunoblotting, (right);

FIG. 5F shows the expression levels of the gluconeogenic genes in the liver; FIG. 5G shows the concentration of glucose in the blood; FIG. 5H shows the mRNA expression levels of lipogenic genes in the liver and epididymal WAT, free fatty acids, liver triglycerides and the lipogenic capacity of adipocytes; FIG. 5I shows total cholesterol, HDL-C and LDL-C concentrations; FIG. 5J shows β-oxidation in the liver using 14C-labeled palmitate; FIG. 5K shows the mRNA expression level of the fatty acid oxidation gene in the epididymal WAT, the triglyceride concentration, and the extent of lipolysis upon stimulation with isoproterenol; FIG. 5L shows hematoxylin-eosin-stained liver tissue; FIG. 5M shows the total ketone body in the blood under feeding and fasting conditions; FIG. 5N shows the mRNA expression level of the ketogenic gene in the liver; and FIG. 5O shows the phosphorylation level of S6 (Ser240/244) and activation of the mTORC1 signaling pathway in liver lysates derived from the freely fed group, the fasted group and the high-fat-diet-fed group of MPK38^{+/+}, MPK38^{+/-} and MPK38^{-/-} female mice;

FIGS. 6A-6G show that MPK38 ameliorates obesity-related disorders in the diet-induced obese male mice, wherein FIG. 6A shows the downregulation of ASK1/TGF-β/p53 signaling in the epididymal WAT derived from 7-month-old MPK38^{-/-} male mice; FIG. 6B shows the size distribution of adipocytes (top) and the hematoxylin-eosin-stained epididymal WAT of 7-month-old high-fat-diet C57BL/6N male mice injected with Ad-MPK38 virus; FIG. 6C shows the mRNA expression level of the adipogenic genes in the epididymal WAT; FIGS. 6D and 6E show the concentration of glucose in the blood (left), glucose and insulin resistance (middle), glucose AUC (area under the curve) during GTT (right of FIG. 6D), and glucose AAC (area above the curve) during ITT; FIG. 6F shows the level of 14C-2-deoxy-glucose uptake in vitro in epididymal WAT and muscle and the expression levels of IRS-PI3K-signaling-pathway-related factors, detected by immunoblotting, after injection of insulin into the inferior vena cava: and FIG. 6G shows the mRNA expression level and glucose concentration of gluconeogenic genes in the liver;

FIGS. 7A-7B show a comparison of ASK1/TGF-β/p53 signal and MPK38 kinase activity and expression levels in male mice fed a standard diet or a high-fat diet, wherein FIG. 7A shows the expression levels of β/p53-signaling-pathway-related factors in hepatocytes derived from C57BL/6N male mice fed a standard diet or a high-fat diet, detected by immunoblotting using each antibody, depending on treatment or non-treatment with, as a ASK1/TGF-β/p53 stimulator, H₂O₂ (2 mM, 30 min), TGF-β1 (100 μM, 20 h) or 5FU (0.38 mM, 30 h); and FIG. 7B shows the result of immunoblotting using a phosphorylation-specific antibody against ZPR9 Thr252 or an in-vitro kinase assay after immunoprecipitation of liver or WAT lysate with each antibody;

FIG. 8 shows the expression of adenovirus-mediated WT MPK38 and kinase-dead (K40R) MPK38 in the liver and WAT and the result of immunoblotting analysis showing the expressions of WT MPK38 and K40R MPK38 in the liver and WAT 16 days after injection of WT MPK38 and K40R MPK38 into the tail and epididymal fat pads of 6-7-month-old middle-aged high-fat-diet-fed male mice using adenovirus;

FIGS. 9A-9H show the effect of induction of MPK38 expression on the amelioration of obesity-related lipid metabolism disorders in diet-induced obese male mice, wherein FIG. 9A shows the concentration of free fatty acids in the blood and the mRNA expression levels of lipogenic genes in the epididymal WAT; FIG. 9B shows the extent of lipolysis of adipocytes stimulated with isoproterenol (left) and the mRNA expression level of the fatty acid oxidation gene in the epididymal WAT; FIG. 9C shows β-oxidation of the liver using 14C-labeled palmitate; FIG. 9D shows blood triglycerides, total cholesterol, HDL-C and LDL-C concentrations; FIG. 9E shows hematoxylin-eosin-stained liver tissue; FIG. 9F shows the total ketone body concentration under feeding and fasting conditions; FIG. 9G shows the mRNA expression level of the ketogenic gene in the liver; and FIG. 9H shows the phosphorylation level of S6 (Ser240/244) (left) and activation of the mTORC1 signaling pathway (right) in liver lysates derived from high-fat-diet-fed male mice not infected or infected with adenovirus, subjected to free feeding, fasting, and feeding for 2 hours after fasting;

FIGS. 10A-10O show that MPK38-expressing 7-month-old high-fat-diet-fed female mice have no effect of ameliorating MPK38-mediated metabolic disorders, wherein FIG. 10A shows the adipocyte size distribution in high-fat-diet-fed female mice injected with adenovirus (top) and hematoxylin-eosin-stained epididymal WAT tissue (bottom); FIG. 10B shows the mRNA levels of adipogenic regulators in the epididymal WAT; FIGS. 10C and 10D shows blood glucose and insulin concentrations under feeding and fasting conditions, and glucose and insulin resistance; FIG. 10E shows the extent of 14C-2-deoxy-glucose uptake in the epididymal WAT and muscle (left) and the expression levels of IRS-PI3K-signaling-related factors, detected by immunoblotting upon treatment with insulin (right); FIG. 10F shows the expression level of the gluconeogenic gene in the liver; FIG. 10G shows the mRNA expression levels of lipogenic genes in the epididymal WAT and the concentration of free fatty acids; FIG. 10H shows the extent of lipolysis in adipocytes stimulated with isoproterenol (top) and the mRNA expression level of the fatty acid oxidation gene in the epididymal WAT (bottom); FIG. 10I shows β-oxidation of the liver using 14C-labeled palmitate; FIG. 10J shows blood triglyceride, total cholesterol, HDL-C and LDL-C concentrations; FIG. 10K shows hematoxylin-eosin-stained liver tissue; FIG. 10L shows total ketone body concentration in the blood under feeding and fasting conditions; FIG. 10M shows the mRNA expression level of the ketogenic gene in the liver; FIG. 10N shows the phosphorylation level of S6 (Ser240/244) (left) and activation of the mTORC1 signaling pathway (right) in liver lysates derived from adenovirus-infected female mice, subjected to free feeding, fasting, and feeding for 2 hours after fasting; and FIG. 10O shows the content of proinflammatory cytokines in the blood (top) and the expression level of the gene in the epididymal WAT (bottom);

FIGS. 11A-11F show the lack of metabolic changes in glucose due to castration of middle-aged MPK38^{-/-} male mice, wherein FIG. 11A shows the adipocyte size distribution (top) and hematoxylin-eosin-stained epididymal WAT tissue (bottom) in each experimental group (4.5 months old and 7 months old) of castrated MPK38^{+/+} male mice, MPK38^{-/-} male mice and sham-operated male mice; FIG. 11B shows the mRNA levels of lipogenic genes; FIGS. 11C and 11D show glucose and insulin resistance; FIG. 11E shows the level of 14C-2-deoxy-glucose uptake in the epididymal WAT and muscle (left) and the expression level of IRS-PI3K-signaling-related factors, detected by immunoblotting, upon treatment with insulin (right); and FIG. 11F shows the mRNA expression level of gluconeogenic genes in the liver;

FIGS. 12A-12F show the lack of metabolic changes due to castration in middle-aged MPK38^{-/-} male mice, wherein FIGS. 12A and 12B show blood glucose and insulin concentrations in the feeding group and the fasting group; FIG. 12C shows the mRNA expression level of the lipogenic gene in the epididymal WAT (top), the concentration of free fatty acids in the blood (bottom left), and the triglyceride content in the liver (bottom right); FIG. 12D shows the total blood cholesterol and HDL-C and LDL-C concentrations; FIG. 12E shows hematoxylin-eosin-stained liver tissue; and FIG. 12F shows the phosphorylation level of S6 (Ser240/244) (left) and the levels of the mTORC1-signaling-pathway-related factors (right) after obtaining liver lysates from groups of castrated mice and sham-operated mice subjected to free feeding, fasting, and feeding for 2 hours after fasting, respectively;

FIGS. 13A-13F show the lack of changes in lipid metabolism due to castration in middle-aged MPK38^{-/-} male mice, wherein FIG. 13A shows the mRNA expression level of the lipogenic gene in the liver (left) and the lipogenic capacity in the hepatocytes (right); FIG. 13B shows the mRNA expression level of the lipogenic gene in the epididymal WAT; FIG. 13C shows β-oxidation of the liver using 14C-labeled palmitate; FIG. 13D shows the mRNA expression level of the fatty acid oxidation gene in the epididymal WAT (left), blood triglyceride concentration (middle) and extent of lipolysis in isoproterenol-stimulated adipocytes (right); FIG. 13E shows the concentration of total ketone bodies in the blood in the feeding group and the fasting group; and FIG. 13F shows the mRNA expression level of the ketogenic gene in the liver;

FIGS. 14A-14H show that testosterone replacement did not affect glucose or lipid metabolism in middle-aged (7 months old) MPK38^{-/-} male mice, wherein FIG. 14A shows glucose and insulin resistance; FIG. 14B shows in-vitro 14C-2-deoxy-glucose uptake depending on treatment with human insulin (100 nM) (left) and the expression levels of IRS-PI3K-signaling-related factors, detected by immunoblotting, upon treatment with insulin, (right); FIG. 14C shows the expression level of the gluconeogenic gene in the liver; FIG. 14D shows the mRNA expression level of the lipogenic gene in the epididymal WAT (left) and the lipogenic capacity of adipocytes (right); FIG. 14E shows the mRNA expression level of the fatty acid oxidation gene in the epididymal WAT (left) and the extent of lipolysis in adipocytes stimulated with isoproterenol (right); FIG. 14F shows the mRNA expression level of lipolytic genes; FIG. 14G shows measurement of hepatic β-oxidation using 14C-labeled palmitate; and FIG. 14H shows the levels of testosterone and luteinizing hormone (LH) in serum detected by ELISA;

FIGS. 15A-15Q show that MPK38 did not affect metabolic changes induced by sterilization in middle-aged female mice, wherein FIG. 15A shows the adipocyte size distribution in ovariectomized and unovariectomized female mice (MPK38^{+/+} or MPK38^{-/-} female mice) (left) and hematoxylin-eosin-stained epididymal WAT tissue (right); FIG. 15B shows the mRNA expression levels of the adipogenic regulators in the epididymal WAT; FIGS. 15C and 15D show glucose and insulin resistance; FIGS. 15E and 15F show blood glucose and insulin concentrations under feeding and fasting conditions; FIG. 15G shows the degree of 14C-2-deoxy-glucose uptake into the epididymal WAT and muscle upon treatment with insulin and the expression levels of IRS-PI3K-signaling-related factors upon insulin treatment, detected by immunoblotting; FIG. 15H shows the mRNA expression level of the gluconeogenic gene in the liver (left) and the concentration of blood glucose (right); FIG. 15I shows the mRNA expression level of lipogenic genes in the liver and epididymal WAT, free fatty acids, hepatic triglycerides, and the lipogenic capacity of adipocytes; FIG. 15J shows total blood cholesterol, HDL-C and LDL-C concentrations; FIG. 15K shows the mRNA expression level of lipolytic enzyme genes;

FIG. 15L shows hepatic β-oxidation using 14C-labeled palmitate; FIG. 15M shows the expression level of a fatty acid oxidation gene in the epididymal WAT (top left), blood triglyceride (top right) and the extent of lipolysis in adipocytes stimulated with isoproterenol (bottom); FIG. 15N shows hematoxylin-eosin-stained liver tissues of ovariectomized and unovariectomized female mice (MPK38^{+/+} or MPK38^{-/-} female mice); FIG. 15O shows the total ketone body concentration in the blood under feeding and fasting conditions; FIG. 15P shows the mRNA expression level of the ketogenic gene in the liver; and FIG. 15Q shows the phosphorylation level of S6 (Ser240/244) and the expression levels of the mTORC1-signaling-pathway-related factors after obtaining hepatocyte lysates from ovariectomized and unovariectomized female mice (MPK38^{+/+} or MPK38^{-/-} female mice) subjected to free feeding, fasting, and feeding for 2 hours after fasting;

FIGS. 16A-16F show the roles of MPK38 on the production of testosterone in male mice, wherein FIGS. 16A and 16B show the concentrations of testosterone and estrogen in the blood in castrated or ovariectomized MPK38^{+/+} and MPK38^{-/-} mice (4.5 months old and 7 months old), detected through LC-MS/MS analysis; and FIGS. 16C to 16F show the mRNA expression levels of genes involved in steroid production in testes (C, D) and ovaries (E, F) of normal-diet-fed MPK38^{+/+} and MPK38^{-/-} mice;

FIG. 17 shows that MPK38 deficiency reduces the production of steroids in mature male mice, and the results of measurement of the mRNA expression levels of the genes involved in steroid hormone synthesis in the testes (top) and ovaries (bottom) of MPK38^{+/+} and MPK38^{-/-} mice fed a standard diet;

FIGS. 18A-18E show the effects of testosterone and estrogen on MPK38 activity and stability, wherein FIG. 18A shows the result of immunoblotting using anti-MPK38 antibody for primary adipocytes of male and female mice treated with DHT or E2 (10 nM each) for 24 hours and then treated with 20 μg/ml of cycloheximide (CHX) alone at regular time intervals, or with 10 μM MG132; FIG. 18B shows a ubiquitinated level of MPK38 in the adipocytes of male and female mice treated with DHT, E2 or vehicle; FIG. 18C shows the extent of formation of the internal MPK38-Mdm2 complex through immunoblotting using anti-Mdm2 antibodies after immunoprecipitation using an anti-MPK38 antibody (IP: α-MPK38) regarding the adipocyte lysate of each experimental group treated with DHT, E2 or a vehicle alone; FIG. 18D shows the extent of formation of a complex of MPK38 and ZPR9 (left) and the extent of formation of a complex of MPK38 and Trx (right) using anti-ZPR9 and anti-Trx antibodies, respectively, after immunoprecipitation using anti-MPK38 antibody for the anti-adipocyte lysate of each experimental group treated with DHT, E2 or vehicle; and FIG. 18E shows changes in the expression of genes involved in MPK38-dependent ASK1/TGF-β/p53 signaling, detected through immunoblotting using each antibody, regarding an adipocyte lysate of an experimental group treated with DHT, E2 or vehicle in the presence or absence of H₂O₂ (2 mM, 30 min), TGF-β1 (100 pM, 20 h) and 5FU (0.38 mM, 30 h) (4.5-month-old female and male mice);

FIGS. 19A-19C show the effects of testosterone and estrogen on liver lipid metabolism and MPK38 kinase activity, detected using mature mouse hepatocytes, wherein FIG. 19A shows mRNA expression levels of lipogenic genes (FAS, SCD1, SREBP1c), fatty acid oxidation genes (PPARα, CPT1, ACO) and lipolytic genes (HSL, ATGL, ADRB3) depending on DHT or E2 (10 nM each) treatment of hepatocytes obtained from 4.5-month-old and 7-month-old female and male mice; FIG. 19B shows β-oxidation of hepatocytes in the group treated with DHT or E2 along with 14C-labeled palmitate and the group not treated therewith, in which the drawing on the left is a non-treatment group and the drawing on the right is a treatment group; and FIG. 19C shows the MPK38 kinase activity detected by immunoblotting using each antibody after treatment of hepatocytes obtained from 4.5-month-old mice with DHT or E2;

FIG. 20 shows that the deficiency of MPK38 decreases the production of testosterone and luteinizing hormone (LH) in mature male mice, wherein the concentration of testosterone (left) and LH (right) in the blood in 4.5-month-old and 7-month-old normal-diet-fed MPK38^{+/+} and MPK38^{-/-} male mice is measured using ELISA; and

FIG. 21 shows a WT-MPK38 recombinant vector map and a K40R-MPK38 recombinant vector map for inducing the expression of MPK38 or K40R-MPK38 in an embodiment of the present invention.

### DETAILED DESCRIPTION OF ILLUSTRATIVE EMBODIMENTS

The present invention is characterized in that it provides a novel use of MPK38 as a therapeutic agent for obesity or metabolic diseases specific to middle-aged men.

Specifically, the present invention is directed to a pharmaceutical composition for preventing or treating obesity or metabolic diseases specific to middle-aged men containing an MPK38 protein or a gene encoding the same as an active ingredient.

Middle-aged obesity and metabolic diseases may occur in both women and men, and the causes of obesity in middle-aged men are different from those in middle-aged women. In the case of middle-aged obesity, it is known that obesity peaks in middle-aged men at the age of 40 to 45 on average and in middle-aged women at the age of 50 to 55 on average, followed by a decrease. In particular, male obesity and metabolic diseases are caused by deteriorations in adrenal secretion function, sperm fertility and testicular cell function, and reductions of serum testosterone level, basal metabolism and muscle mass. On the other hand, middle-aged women experience irregular menstrual volume or cycle due to a deficiency of ovarian function during climacterium before and after menopause, and undergo menopause due to decreased estrogen secretion, and exhibit promoted adrenal cortex function due to malfunction of the anterior pituitary gland, resulting in masculinization, as well as thyroid malfunction due to the influence of thyroid hormones, which leads to obesity and metabolic diseases.

Meanwhile, as a result of extensive research to develop effective therapeutic agents for obesity and metabolic diseases that occur in middle-aged men, the present inventors found that obesity, abnormalities in lipid metabolism and energy metabolism, and increased expression of inflammation-associated factors appear in MPK38-deficient middle-aged male mice. In addition, the present inventors found that this phenomenon does not appear in female mice, and occurs specifically in male mice, especially in middle-aged male mice.

Accordingly, the present inventors identified the relationship between middle-aged male obesity and metabolic disease and MPK38 through the experiments performed in the examples described later. According to an embodiment of the present invention, MPK38-knockout MPK38^{-/-} mice were produced from 7-month-old male and female mice through a gene trap insertion method, and then obesity, changes in sugar, lipid and energy metabolisms, and induction of inflammation were analyzed on the MPK38-knockout MPK38^{-/-} mice as well as normal mice not deficient in MPK38.

The result of visual inspection showed that MPK38-knockout MPK38^{-/-} mice became remarkably enlarged compared to normal mice, indicating obesity. This phenomenon occurred only in male mice, but did not occur in MPK38-knockout female mice.

The result of glucose-metabolism-related analysis also showed that MPK38^{-/-} male mice exhibited glucose resistance, had lower insulin sensitivity than normal mice, and exhibited an increase in the expression of glucose production-related genes compared to normal mice. In addition, MPK38^{-/-} male mice exhibited increased free fatty acid (FFA), triglycerides, total cholesterol, high-density lipoprotein-cholesterol (HDL-C), and low-density lipoprotein-cholesterol (LDL-C) in the blood and an increase in expression of lipogenesis-related genes compared to normal mice.

In addition, MPK38^{-/-} male mice exhibited increased production of inflammation-related cytokines and a reduction in energy metabolism, such as energy consumption, 24-hour O₂ consumption, CO₂ production and respiratory exchange rate (RER) compared to normal mice.

In addition, surprisingly, the above-described phenomena occurring in MPK38^{-/-} male mice were not observed in MPK38^{-/-} female mice.

Therefore, the present inventors identified that MPK38 is directly related to middle-aged male obesity and metabolic diseases using 7-month-old mice, corresponding to middle-aged (40-45 years old) humans, and that obesity and metabolic disorders are induced by MPK38 deficiency in middle-aged male mice. As a result, it is expected that when the expression of MPK38 is induced or the activity thereof is enhanced, middle-aged male obesity and metabolic diseases can be ameliorated or treated.

Furthermore, in another embodiment of the present invention, the relationship between MPK38 and ASK1/TGF-β/p53 signaling with respect to middle-aged male-specific obesity and metabolism (sugar and lipid metabolisms) was analyzed. The result showed that 7-month-old MPK38^{-/-} male mice exhibited a decrease in MPK38-dependent ASK1/TGF-β/p53 signaling activity compared to normal mice of the same age and gender, and obese male mice (ob/ob) and high-fat-diet-fed male mice also exhibited a phenomenon similar thereto.

Therefore, it was found that MPK38-dependent ASK1/TGF-β/p53 signaling activity greatly affects middle-aged male obesity and metabolic disease, and that MPK38 deficiency causes obesity and metabolic disorders due to inhibited or reduced ASK1/TGF-β/p53 signaling activity.

Accordingly, the present inventors introduced a wild-type (WT) MPK38 (Ad-MPK38) gene into high-fat-diet-fed 7-month-old male mice, corresponding to middle-aged men, using adenovirus to induce expression in order to determine whether or not induction of expression of MPK38 can actually ameliorate or treat middle-aged male-specific obesity and metabolic disorders. At this time, a K40R MPK38 (Ad-K40R) gene, from which the kinase activity of MPK38 was lost, was also introduced as a control to induce expression. As a result, the group introduced with WT MPK38 exhibited decreased size of adipocytes, decreased expression of adipogenic proteins, lowered glucose and insulin concentrations in the blood, and increased glucose tolerance and insulin sensitivity. In addition, MPK38 expression induction resulted in a decrease in the concentration of free fatty acids in the blood and a decrease in the expression of lipogenic genes, but an increase in the expression of genes related to lipolysis and fatty acid oxidation.

However, these results did not appear in the group introduced with the K40R MPK38 gene or in female mice.

Therefore, based on these results, the present inventors found that MPK38 can prevent, ameliorate and treat obesity and metabolic diseases specific to middle-aged men.

Furthermore, the present inventors identified that MPK38 is involved in obesity, and glucose and lipid metabolisms specific to middle-aged men, and, based thereon, determined whether or not MPK38 is also involved in the production of testosterone, a male hormone.

The result showed that the content of testosterone in uncastrated 7-month-old MPK38^{-/-} male mice was similar to that of a normal male mouse group of the same age, and that there was no significant difference in estrogen content therebetween.

In addition, the expression levels of steroid-producing genes were analyzed in 7-month-old normal male mice and MPK38^{-/-} male mice fed a standard diet. The result showed that the expression of genes involved in the production of testosterone, a male hormone, was decreased in 7-month-old MPK38^{-/-} male mice, and surprisingly, this phenomenon was not observed in 4.5-month-old MPK38^{-/-} male mice or normal male mice.

In other words, these results mean that MPK38 has no effect on the production of estrogen, a female hormone, but is involved in the production of testosterone in middle-aged men, and that induction of expression of MPK38 or promotion of activity thereof can enhance the production of testosterone, which is decreased in middle-aged men.

Accordingly, the present invention provides a pharmaceutical composition for preventing or treating middle-aged male obesity or metabolic diseases containing an MPK38 protein or a gene encoding the same as an active ingredient.

In the present invention, the MPK38 protein may have the amino acid sequence of SEQ ID NO: 1, and the gene encoding the MPK38 protein may have the nucleotide sequence of SEQ ID NO: 2.

In addition, the MPK38 gene contained in the composition of the present invention may be incorporated into the composition in a form of being inserted into an expression vector capable of expressing the gene.

The vector that can be used in the present invention is not limited, but is typically a plasmid, phage, cosmid, viral vector, or other medium known in the art. In addition, in the present invention, the MPK38 gene may be isolated from nature or artificially synthesized, and the nucleotide sequence encoding the MPK38 protein may be modified by substitution, deletion or insertion of one or more nucleic acid bases. The protein expressed by this modification should not have a remarkable change in biological functionality thereof. The modification include modifications to heterologous and homologous genes.

The expression vector according to the present invention may be introduced into cells using a method known in the art. For example, the method may include, but is not limited to, transient transfection, microinjection, transduction, cell fusion, calcium phosphate precipitation, liposome-mediated transfection, DEAE dextran-mediated transfection, polybrene-mediated transfection, electroporation, gene gun and other known methods for introducing nucleic acids into cells (Wu et al., J. Bio. Chem., 267:963-967, 1992; Wu and Wu, J. Bio. Chem., 263:14621-14624, 1988).

In one embodiment of the present invention, an adenovirus vector including the MPK38 gene was used.

The adenovirus vector including the MPK38 gene of the present invention may have a structure in which the MPK38 gene is operably linked to a promoter.

The term “operably linked” means that a gene to be expressed is linked to a regulatory sequence thereof to enable the expression of the gene, and the term “promoter” means a DNA sequence capable of regulating transcription of a specific nucleotide sequence into mRNA when linked to the specific sequence. Typically, the promoter is not applied in all cases, but is present in a 5′ direction (i.e., upstream) of the target nucleotide sequence to be transcribed into mRNA, and provides a site to which RNA polymerase and other transcription factors for initiating transcription specifically bind.

The promoter of the present invention may be a constitutive or regulatory promoter, and is preferably a constitutive promoter. The term “constitutive” used in connection with the promoter means that the promoter is capable of instructing the transcription of an operably linked nucleic acid sequence even in the absence of stimulus (e.g., heat shock, chemicals and the like). Meanwhile, the term “regulatory” promoter means a promoter capable of instructing the transcription level of an operably linked nucleic acid sequence in the presence of stimulus (e.g., heat shock, chemicals and the like), unlike the case of absence of stimulus.

The pharmaceutical composition according to the present invention may include a pharmaceutically acceptable carrier in addition to the active ingredient, and examples of the carrier include, but are not limited to, carriers commonly used in formulations, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, and the like. The pharmaceutical composition of the present invention may further contain, in addition to the ingredients described above, a lubricant, a wetting agent, a sweetening agent, a flavoring agent, an emulsifying agent, a suspending agent, a preservative, and the like. Suitable pharmaceutically acceptable carriers and formulations are described in detail in Remington's Pharmaceutical Sciences (19th ed., 1995).

A suitable dosage of the pharmaceutical composition of the present invention may be formulated in various ways depending on factors such as the formulation method, mode of administration, and age, weight, gender, pathological condition, diet, administration time, route of administration, excretion rate, and responsiveness of the patient. Meanwhile, the dosage of the pharmaceutical composition of the present invention is preferably 0.0001 to 100 mg/kg (body weight) per day.

The pharmaceutical composition of the present invention may be administered orally or parenterally, and the parenteral administration may be topical application to the skin, intravenous injection, subcutaneous injection, intramuscular injection, intraperitoneal injection, transdermal administration, or the like. The concentration of the active ingredient contained in the composition of the present invention may be determined in consideration of the purpose of treatment, the condition of the patient, the required administration period, or the like, and is not limited to a specific concentration range.

The pharmaceutical composition of the present invention may be prepared in a unit dosage form, or may be prepared by injection into a multi-dose container by formulation using a pharmaceutically acceptable carrier and/or excipient according to a method that can be easily implemented by those skilled in the art. In this case, the formulation may be in the form of a solution, suspension, or emulsion in an oil or aqueous medium, or may be in the form of an extract, powder, granule, tablet or capsule, and may further contain a dispersant or a stabilizer.

As used herein, the term “treatment” means reversing or ameliorating a disease or disorder to which the treatment is applied, or one or more symptoms of the disease or disorder, or suppressing or preventing the progression thereof, unless otherwise mentioned herein.

In the present invention, the disease may be obesity or a metabolic disease, preferably obesity or a metabolic disease that occurs in middle-aged men.

MPK38 contained in the composition of the present invention, which is specific for middle-aged men, reduces the size of adipocytes, inhibits the expression of the adipogenic genes C/EBPα (CCAAT-enhancer-binding protein α) and PPARγ (peroxisome proliferator-activated receptor gamma and FABP4), improves insulin sensitivity, reduces blood glucose and insulin, reduces blood triglyceride, total cholesterol, HDL-C and LDL-C levels, and increases the ketone body formation, thereby preventing or treating middle-aged male obesity or metabolic diseases.

In addition, MPK38 of the present invention can enhance the production of testosterone, and further, the composition of the present invention can be used as a composition for preventing, ameliorating or treating testosterone deficiency.

The metabolic disease may include, but is not limited to, diabetes, hyperlipidemia, arteriosclerosis, high blood pressure, cardiovascular diseases, fatty liver, obesity-derived inflammatory diseases, obesity-derived autoimmune diseases, obesity-derived cancer, and these diseases may occur in middle-aged men.

Also, the present invention provides a health functional food for preventing or ameliorating obesity or metabolic diseases specific to middle-aged men containing an MPK38/MELK protein or a gene encoding the same as an active ingredient.

The health functional food of the present invention may have a formulation such as a tablet, capsule, pill or liquid.

The health functional food according to the present invention may further include a cytologically acceptable carrier, and there is no particular limitation as to the kind of food. Examples of the food to which the substance described above can be added include meat, sausages, bread, chocolate, candy, snacks, confectioneries, pizza, ramen, other noodles, gum, dairy products including ice cream, various soups, beverages, tea, drinks, alcoholic beverages, vitamin complexes and the like, and include all common health foods. A description of a specific cooking method or production method thereof will be obvious to those skilled in the art of the present invention, and will be omitted. In addition, the cytologically acceptable carrier may be the aforementioned pharmaceutically acceptable carrier.

When the health functional food of the present invention is a beverage composition, it may contain various flavoring agents or natural carbohydrates or the like as additional components, like a common beverage. The natural carbohydrates described above may be monosaccharides such as glucose and fructose, disaccharides such as maltose and sucrose, polysaccharides such as dextrin and cyclodextrin, or sugar alcohols such as xylitol, sorbitol, and erythritol. The sweetener may be any of natural sweeteners, such as thaumatin and stevia extracts, and synthetic sweeteners, such as saccharin and aspartame. The content of the natural carbohydrate is generally about 0.01 to about 0.04 g, preferably about 0.02 to about 0.03 g, with respect to 100 ml of the composition of the present invention.

In addition, the composition of the present invention includes a variety of nutrients, vitamins, electrolytes, flavoring agents, colorants, pectic acids and salts thereof, alginic acid and salts thereof, organic acids, protective colloidal thickeners, pH adjusters, stabilizers, preservatives, glycerin, alcohols, and carbonation agents used in carbonated beverages. In addition, the composition of the present invention may contain flesh for the preparation of natural fruit juice, fruit juice beverage and vegetable beverage. These components may be used independently or in combination. Although the ratio of these additives is not particularly important, it is generally selected from 0.01 to 0.1 parts by weight with respect to 100 parts by weight of the composition of the present invention.

Furthermore, the present invention provides a composition for promoting testosterone production containing an MPK38 protein or a gene encoding the same as an active ingredient.

Testosterone (TS) is essential for the normal growth and development of male sexual and reproductive organs, including seminal vesicles, scrotum, prostate, testis and penis and promotes the development of masculine traits such as laryngeal hypertrophy, vocal cord thickening, hair patterns, body fat distribution, bone mass, and muscle tissue. Normal TS levels regulate vasodilation, muscle mass and volume, reproductive function, sexual relations, spermatogenesis, and the like, and play important mental and physiological roles. TS is synthesized mainly in intracellular mitochondria through the synthesis stage of other steroid hormones such as 30-hydroxysteroid dehydrogenase (3β-HSD) using cholesterol as a raw material (precursor) in Leydig cells, which are interstitial cells in the testes, upon stimulation using a luteinizing hormone, and the level thereof peaks in the late teens to early 20s and then naturally decreases as aging progresses.

This decrease in testosterone hormone causes a testosterone deficiency in middle-aged men, which may cause various problems in the body.

Testosterone deficiency may be caused by diseases of the hypothalamus, pituitary gland or testes, may often accompany aging, and may have a wide range of symptoms including libido loss, erectile dysfunction, low sperm count, aspermia, gradual loss of muscle mass, decreased physical strength, increased abdominal fat, decreased bone density, fatigue, decreased memory and depression.

Meanwhile, MPK38 of the present invention can promote the production of testosterone, the level of which is decreased in middle-aged men, and thus can ameliorate or treat symptoms caused by reduced testosterone levels.

Hereinafter, the present invention will be described in more detail with reference to examples. The examples are provided only for illustration of the present invention, and should not be construed as limiting the scope of the present invention.

<Reagent and Experimental Method>

Antibody

The antibodies used in the experiments of Examples are as follows.

Anti-phospho-S6 (S240/244) (#5364), anti-phospho-mTOR (S2448) (#5536), anti-phospho-ASK1 (T845) (#3765), anti-phospho-MKK3/6 (S189/207) (#9231), anti-phospho-p38 (T180/Y182) (#4511), anti-phospho-ATF2 (T71) (#24329), anti-phospho-S6K1 (T389) (#9206), anti-phospho-4EBP1 (T37/46) (#2855), anti-phospho-AKT1 (T308) (#2965), anti-phospho-AKT1 (S473) (#9018), anti-phospho-AMPKα(Thr172) (#2531), anti-phospho-ACC1 (Ser79) (#11818), anti-mTOR (#2972), anti-S6K1 (#9202), anti-4E-BP1 (#9644), anti-AKT1 (#2938), anti-AMPKα (#5832) and anti-ACC1 (#4190) antibodies were purchased from Cell Signaling Technology (Danvers, Mass.). In addition, anti-phospho-IRO(Y1322) (#04-300) and anti-In (MABN390) antibodies were purchased from Millipore Corp. (Bedford, Mass.). In addition, anti-phospho-IRS1 (Y989) (sc-17200), anti-IRS1 (sc-8038), anti-p53 (DO-1) (sc-126), antip21 (sc-6246), anti-Mdm2 (sc-965), anti-PAI-1 (sc-5297), anti-ASK1 (sc-390275), anti-S6 (sc-74459), anti-MKK3 (sc-271779), anti-ATF2 (sc-242), anti-p38 (sc-7972), anti-Bax (sc-7480), anti-Trx (sc-271281), anti-CDK4 (sc-23896) and anti-Cyclin D1 (sc-8396) antibodies were purchased from Santa Cruz Biotechnology (Santa Cruz, Calif.). Anti-phospho-Ser/Thr (ab17464), anti-GLUT1 (ab652), and anti-GLUT4 (ab654) antibodies were purchased from Abcam (Cambridge, UK). Anti-Smad7 antibody (MAB2029) was purchased from R&D Systems (Minneapolis, Minn.) and anti-β-actin antibody (A2228) was purchased from Sigma (St. Louis, Mo.). F4/80-PE (1:100, #123110), Cd206-Alexa Fluor 647 (1:100, #141708) and Cd11c-FITC (1:100, #117306) were purchased from BioLegend (San Diego, Calif.), and anti-MPK38 and anti-ZPR9 antibodies for detecting the recombinant full-length MPK38 and ZPR9 proteins, respectively, were purchased from Young In Frontier Co. Ltd. (Seoul, Korea).

Oligonucleotide (Primer)

The oligonucleotides (primers) used in the experiments of Example are as follows.

The primers that were used for real-time PCR for the target gene were set forth in Table 1 below.

TABLE 1

| Gene | Sequence | |
|---|---|---|
| 18S rRNA (mouse) | Sense | 5′-GTAACCCGTTGAACCCCATT-3′ |
| | Antisense | 5′-CCATCCAATCGGTAGTAGCG-3′ |
| ACO (mouse) | Sense | 5′-GCCAATGCTGGTATCGAAGAA-3′ |
| | Antisense | 3′-GGAATCCCACTGCTGTGAGAA-3′ |
| ADRB3 (mouse) | Sense | 5′-TGAAACAGCAGACAGGGACA-3′ |
| | Antisense | 5′-GGATGTCCATACCAGGGCAC-3′ |
| ATGL (mouse) | Sense | 5′-CTGAGAATCACCATTCCCACATC-3′ |
| | Antisense | 5′-CACAGCATGTAAGGGGGAGA-3′ |
| CPT1 (mouse) | Sense | 5′-ACCACTGGCCGAATGTCAAG-3′ |
| | Antisense | 5′-AGCGAGTAGCGCATGGTCAT-3′ |
| FAS (mouse) | Sense | 5-TGCTCCAGGGATAACAGC-3 |
| | Antisense | 5′-CCAAATCCAACATGGGACA-3′ |
| G6PC (mouse) | Sense | 5′-TGGTAGCCCTGTCTTTCTTTG-3′ |
| | Antisense | 5′-TTCCAGCATTCACACTTTCCT-3′ |
| HSL (mouse) | Sense | 5′-TTCTCCAAAAGCACCTAGCCAA-3′ |
| | Antisense | 5′-TGTGGAAAACTAAGGGCTTGTTG-3′ |
| PCK1 (mouse) | Sense | 5′-ATCACCGCATAGRCRCRGAA-3′ |
| | Antisense | 5′-ACACACACACATGCTCACAC-3′ |
| PGC1α (mouse) | Sense | 5′-AGCACTCAGAACCATGCAGCAAAC-3′ |
| | Antisense | 5′-TTTGGTGTGAGGAGGGTCATCGTT-3′ |
| PPARα (mouse) | Sense | 5′-CTGCAGAGCAACCATCCAGAT-3′ |
| | Antisense | 5′-GCCGAAGGTCCACCATTTT-3′ |
| SCD1 (mouse) | Sense | 5′-ACCTGCCTCTTCGGGAATTTT-3′ |
| | Antisense | 5′-GTCGGCGTGTGTTTCTGAGA-3′ |
| SREBP1 (mouse) | Sense | 5′-AGCTGCGTGGTTTCCAACA-3′ |
| | Antisense | 5′-CCTCATGTAGGAATACCCTCCTCAT-3′ |
| TNF-α (mouse) | Sense | 5′-ACGGCATGGATCTCAAAGAC-3′ |
| | Antisense | 5′-AGATAGCAAATCGGCTGACG-3′ |
| IL-6 (mouse) | Sense | 5′-GTCCTTCCTACCCCAATTTCCA-3′ |
| | Antisense | 5′-TAACGCACTAGGTTTGCCGA-3′ |
| IL-1β (mouse) | Sense | 5′-TGTGAAATGCCACCTTTTGA-3′ |
| | Antisense | 5′-GGTCAAAGGTTTGGAAGCAG-3′ |
| MCP1 (mouse) | Sense | 5′-CCACTCACCTGCTGCTACTCA-3' |
| | Antisense | 5′-TGGTGATCCTCTTGTAGCTCTCC-3′ |
| Cyp11a1 (mouse) | Sense | 5′-CCTATTCCGCTTTTCCTTTGAGTCC-3' |
| | Antisense | 5′-CGCTCCCCAAATATAACACTGCTG-3′ |
| Cyp17a1 (mouse) | Sense | 5′-TCGGCCCCAGATGGTGACTC-3′ |
| | Antisense | 5′-TGGTCCGACAAGAGGCCTAGAG-3′ |
| 17β-hsd (mouse) | Sense | 5′-AGTGTGGGAGGCTTGATGGGA-3′ |
| | Antisense | 5′-CACTTCGTGGAATGGCAGTCC-3′ |

In addition, the sequences of the PCR primers for recombinant adenovirus were set forth in Table 2 below.

TABLE 2

| Gene | Sequence | |
|---|---|---|
| MPK38 | Sense | 5′-GTAACTATAACGGTCATGAAAGATTATGA |
| | | CGAACTCCTCAAA-3′ |
| | Antisense | 5′-ATTACCTCTTTCTCCTCACATCTTGCAGC |
| | | CAGACAAGAT-3 |

Quantitative Real-Time PCR (qPCR)

Total RNA was extracted from cells and tissues using a TRIzol RNA extraction system (12183555; Invitrogen) according to the manufacturer's instructions, and single-stranded cDNA was synthesized. The synthesis of cDNA was performed using a Superscript cDNA kit (11917010; Invitrogen). qPCR was performed using a LightCycler reaction kit (2239264; Roche Diagnostics). A total of 20 μl of a reaction solution including 2 μl cDNA, 200 nM primers, Taq DNA polymerase, 2.3 mM MgCl₂, and 2 μl SYBR green reagent was prepared, was heat-denatured at 95° C. for 15 minutes, and was then subjected to qPCR by repeating the reaction at 95° C. for 10 seconds, 55° C. for 20 seconds, and 72° C. for 30 seconds a total of 40 times. In addition, all samples were tested in duplicate using 18S ribosomal RNA for normalization of mRNA expression.

Preparation of Experimental Animals

Male and female C57BL/6N mice (14-15 weeks old) purchased from Central Lab. Animal Inc. (Seoul, Korea) were fed a high-fat diet (HFD; Research Diets, Inc. D12492, 60% kcal fat) for 12 weeks and then used in adenovirus mediation experiments. The mice were provided with water and feed in the presence a light/dark cycle of 12 hours and 12 hours in pathogen-free cages. All animal experiments were performed with the approval of the Chungbuk National University Committee (CBNUA-966-16-02) and the Chungbuk National University Animal Care Committee in accordance with the guidelines and regulations established in the ethical review.

MPK38 Knockout Mouse

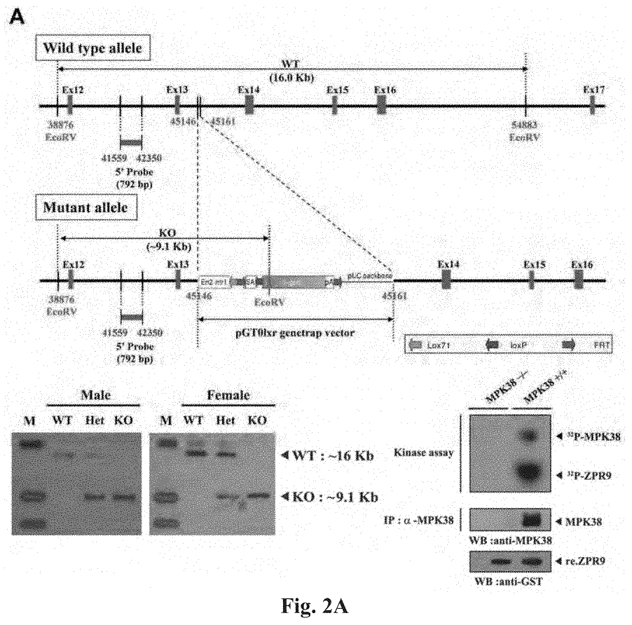

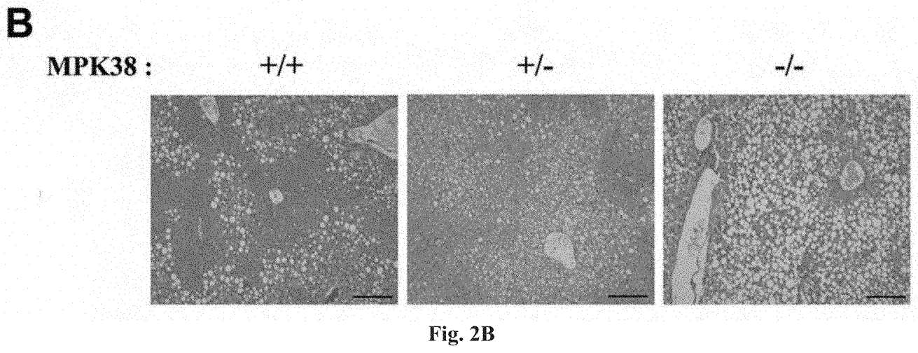

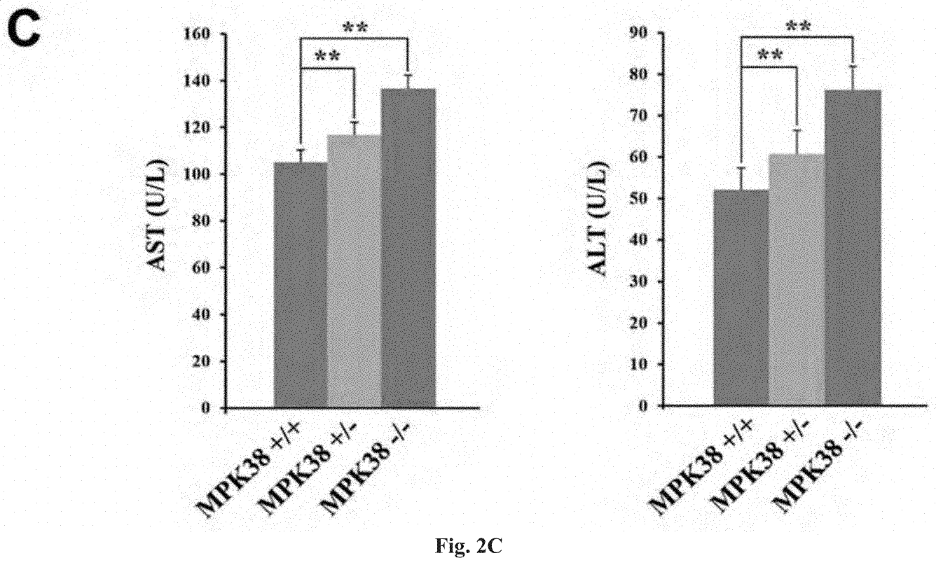

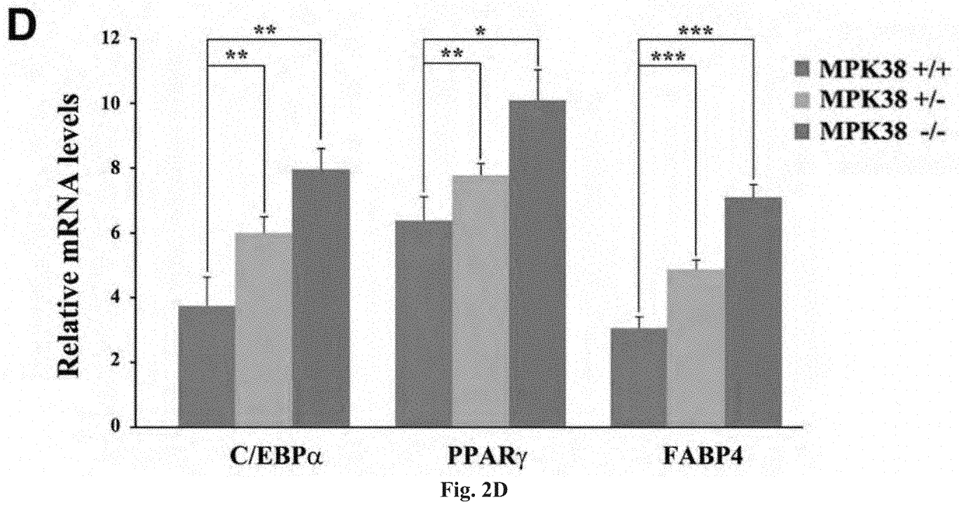

MPK38 knockout (MPK38^{-/-}) mice were produced using C57BL/6N mouse embryonic stem (ES) cells having a gene trap inserted thereinto (AR081, Mutant Mouse Regional Resource Center, UC Davis, USA). Specifically, a schematic diagram showing the preparation of MPK38 knockout mice is shown in FIG. 2A. Exons (Ex) 13-14 were inserted into the gene trap vector, and the EcoRV site was introduced at the target MPK38 gene site. The ˜16 kb and ˜9.1 kb EcoRV fragments indicated by the lines in FIG. 2A represent wild-type and mutant alleles, respectively.

Castration and Ovariectomy of Mice

A mixture of ketamine (34 mg/kg), xylazine (6.8 mg/kg) and acepromazine (1.1 mg/kg) was dissolved in saline, and the mice were anesthetized using this solution, and then surgery was performed. The surgical site was shaved and washed, and then sterilized with 70% alcohol. For castration, after abdomen midline incision, castration was performed such that the testes and attached fat pads were left intact. The testes were removed through an incision, but the testicular ducts and blood vessels supplying the testes were ligated with sterile silk suture and the testicular fat pads were put back, followed by closure with sterile silk suture. For sham castration, the testes with attached fat pads were exposed and put back without organ extirpation after abdominal incision, and the incision was closed with sterile silk suture. For ovariectomy, after midline abdominal incision, the uterus, oviducts, and ovaries with the fat pads were left intact. The ovaries were removed through an incision, but the oviduct and blood vessels supplying the ovaries were ligated with sterile silk suture and the ovarian fat pads were put back, followed by closure with sterile silk suture. For sham ovariectomy, the oviducts, uterus, and ovaries with the fat pads were exposed and put back without organ extirpation after abdominal incision. After surgery, mice were monitored until they are fully recovered from anesthesia. Mice were used for the experiments 5-6 weeks after the surgery.

Glucose and Insulin Resistance Analysis (GTTs/ITTs)

For blood glucose and insulin resistance analysis, after injection of the sample into each mouse, blood was collected from the tail thereof at 0, 10, 20, 30, 45, 60, 90 and 120 minutes following injection using an Accu-Check blood glucose meter (6870228; Roche), and blood glucose was tested. In addition, the results are shown according to the recommendations of the Mouse Metabolic Phenotyping Center. The level of insulin was measured using an ELISA kit (90080; Chrystal Chem.) and the blood glucose level was assayed using an Accu-Check glucometer.

Lipolysis and Lipogenesis Analysis

For lipolysis measurement, the glycerol content was measured based on absorbance at 540 nm using a Free Glycerol Determination Kit (FG0100; Sigma). Isoproterenol-stimulated lipolysis was corrected for basal lipolysis by subtracting the amount of glycerol release in the absence of isoproterenol. Lipogenesis was measured by a method known in the art, and the incorporated radiolabeled glucose was measured using liquid scintillation counting, and normalized by total lipid content.

Assessment of Metabolic Parameters

Serum levels of insulin and free fatty acid were measured by enzymatic linked immunosorbent assay (ELISA) kits (for insulin, Millipore (EZRMI-13K); for free fatty acids, Merck (MAK044)). Serum levels of triglycerides, total cholesterol, HDL- and LDL-cholesterol and glucose were measured using a Hitachi automatic analyzer 7080 (Hitachi Science System Ltd., Ibaraki, Japan). The serum triglyceride measurement kit (TR0100; Sigma) was also used to measure the level of triglyceride in the liver. Total ketone levels were quantified using an enzyme colorimetric assay kit (415-73301 & 411-73401; Wako, Richmond, Va.), and fatty acid oxidation was measured in fresh liver and WAT homogenates.

Replacement of Testosterone and Measurement of Testosterone/Estrogen/Luteinizing Hormone (LH)

For testosterone replacement, testosterone pellets (25 μg/day, SA-151; Innovative Research of America) or placebo pellets (25 μg/day, SC-111; Innovative Research of America) were subcutaneously injected into the castrated mouse group.

In addition, levels of testosterone and estrogen in the serum were analyzed using UHPLC-MS-MS coupled to an Agilent 6490 triple quadrupole (QqQ). The analysis using the Agilent 6490 triple quadrupole (QqQ) mass spectrometer was performed according to the protocol set forth by McLeod at al. The level of luteinizing hormone (LH) was detected using ELISA kits (E-EL-M3053; Elabscience). The quantification of LH levels was obtained using a microplate reader at 550 nm with the correction wavelength set to 450 nm.

Flow Cytometry of Macrophages in Adipose Stromal Vascular Fraction (SVF)

SVF cells were analyzed using propidium iodide (PI) (P4864; Sigma) and BD LSRFortessa Cell Analyzer (BD Biosciences), and data were analyzed using FlowJo software version X.0.7 (Tree Star, Ashland, Oreg.).

Production of Recombinant Adenovirus for Inducing MPK38 Expression

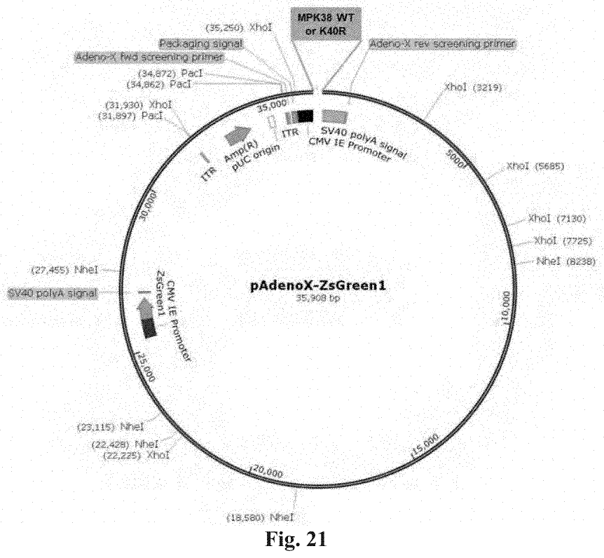

Recombinant adenovirus (wild type, WT; kinase-dead, K40R) expressing MPK38 was produced by PCR using, as templates, pEBG-MPK38 plasmids (WT and K40R), the primers set forth in Tables 1 and 2 above, and Advantage HD polymerase mix (Clontech, #639241). In addition, the WT-MPK38 recombinant vector map and the K40R-MPK38 recombinant vector map used for the production of the recombinant adenovirus are shown in FIG. 21.

Statistical Analysis

Analysis values were expressed as mean±S.E.M., and the analysis results were obtained by performing at least three independent trials, unless otherwise indicated. Statistical analysis was performed by one-way or two-way ANOVA, followed by Tukey's multiple comparison analysis using GraphPad Prism 7.0 software (GraphPad software).

### Example 1

Confirmation of Induction of Obesity Specific to Middle-Aged Men Due to MPK38 Deficiency, of Abnormalities in Sugar, Lipid and Energy Metabolisms, and of Inflammation

In order to ascertain the function of MPK38 in vivo, MPK38^{-/-} mice were produced using a gene trap insertion method (see FIG. 2A). Then, a standard diet was supplied to the mice, and the degree of obesity, glucose, lipid and energy metabolisms, and inflammation were analyzed in 6-7-month-old normal mice and MPK38^{-/-} mice.

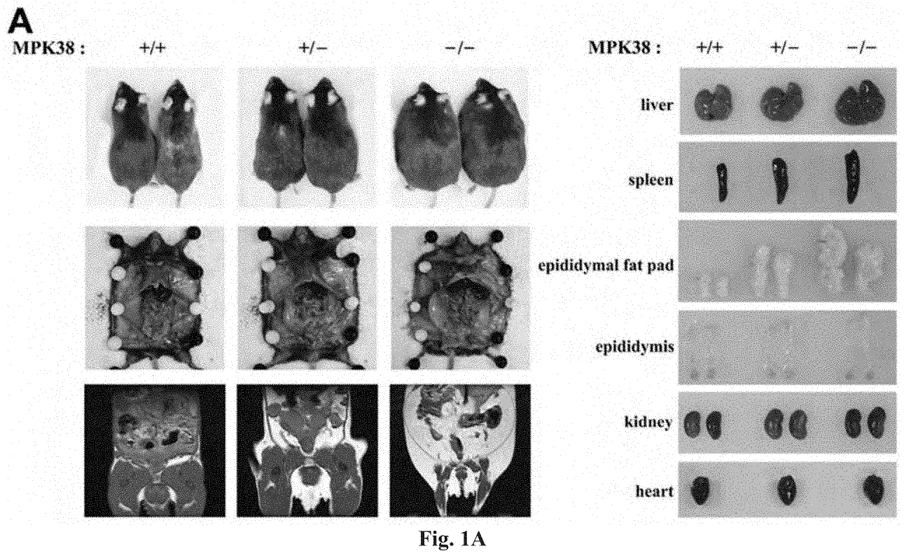

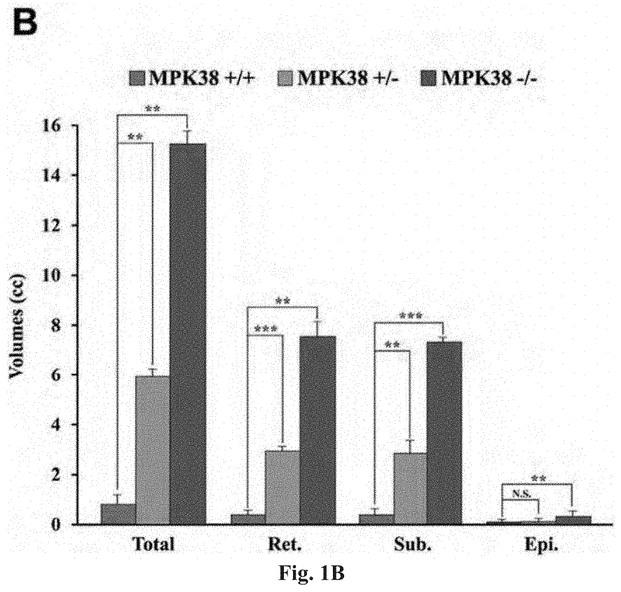

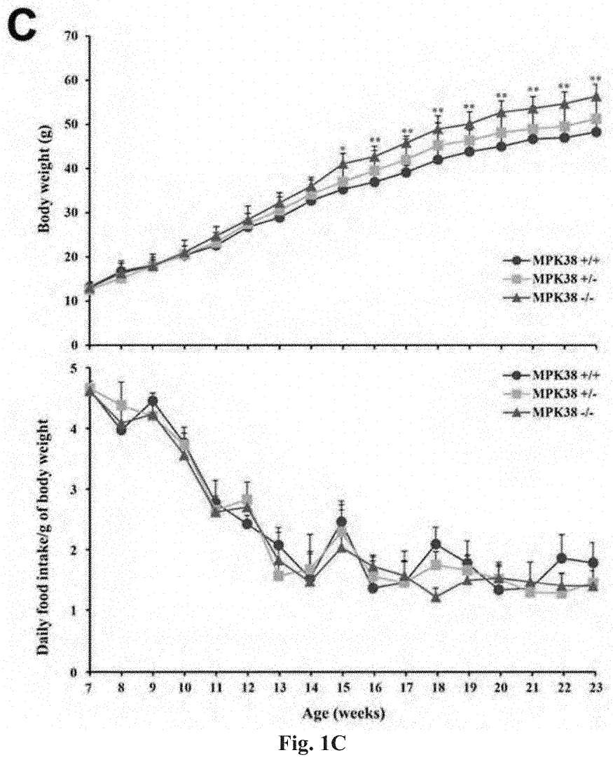

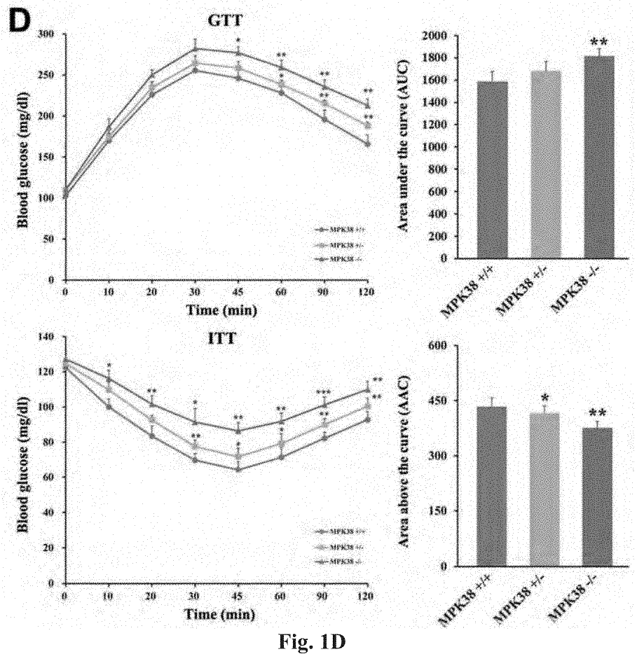

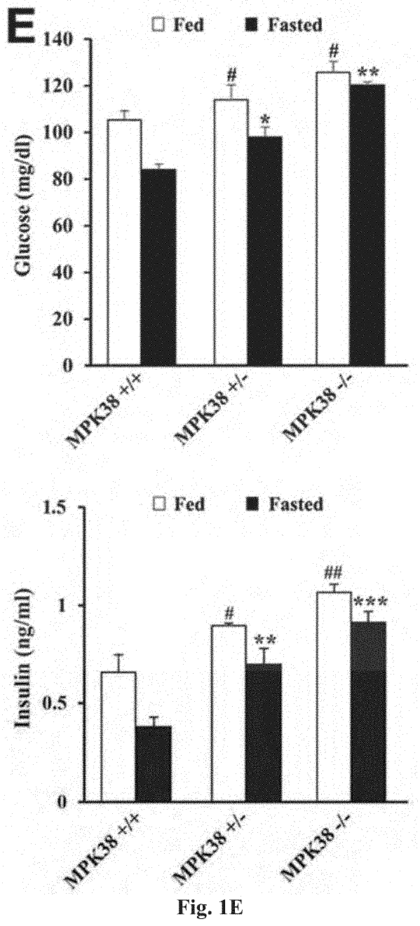

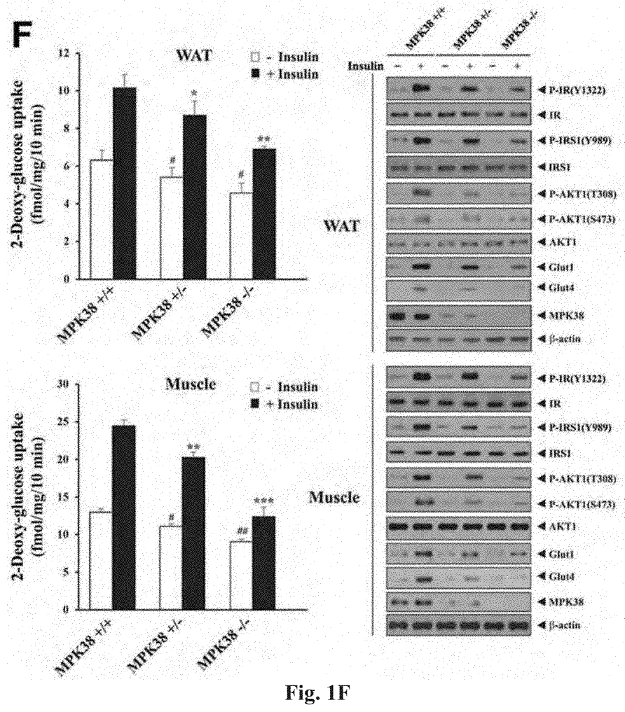

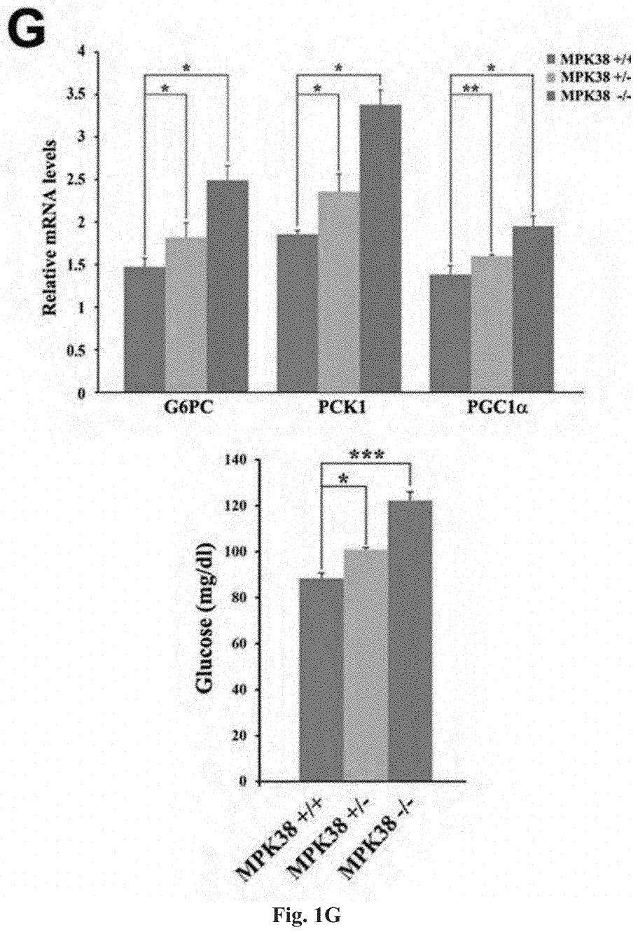

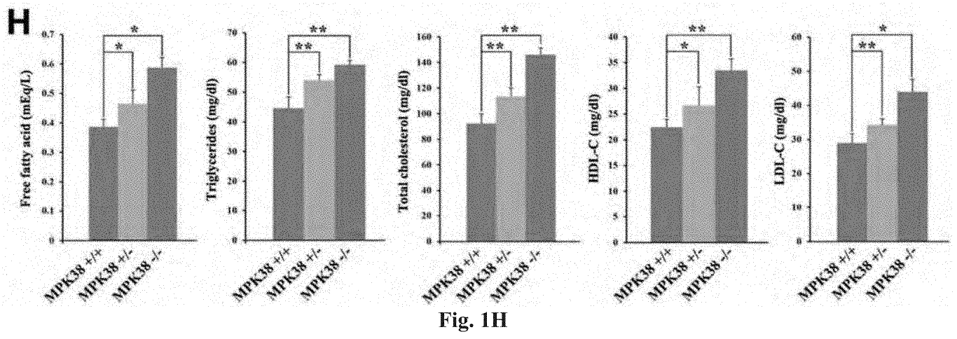

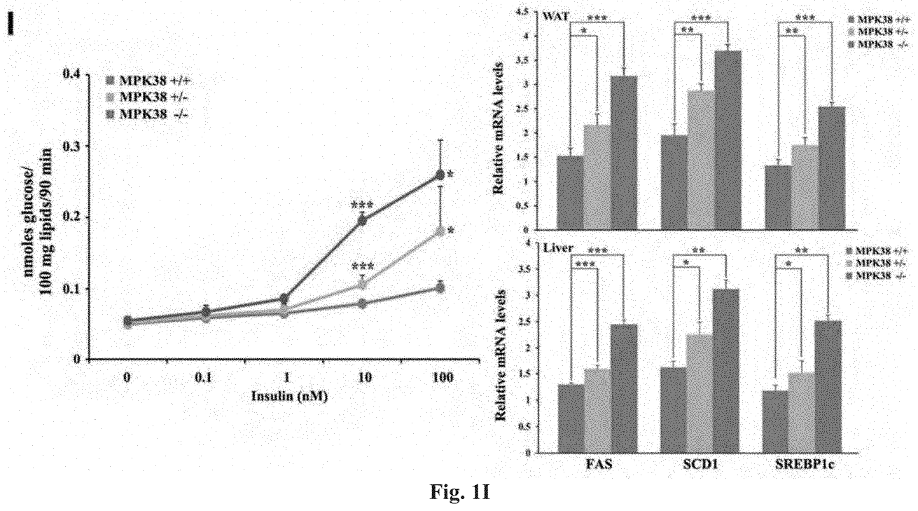

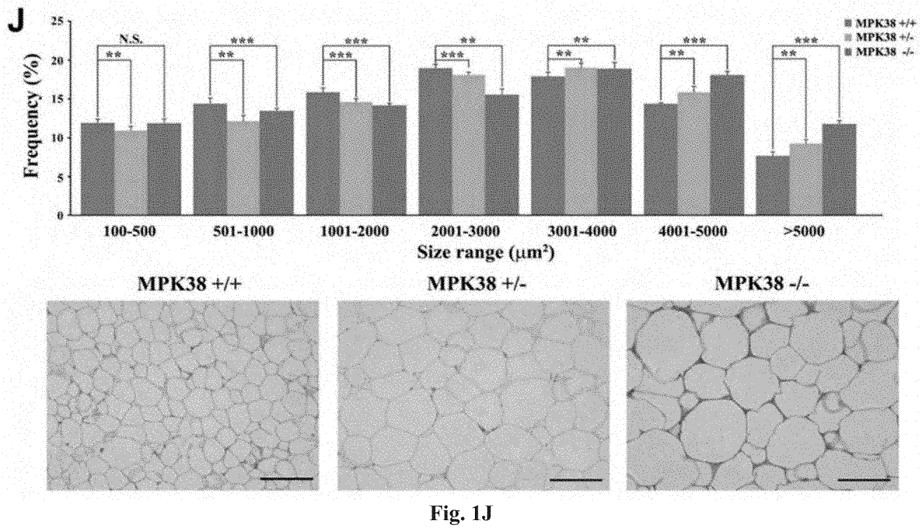

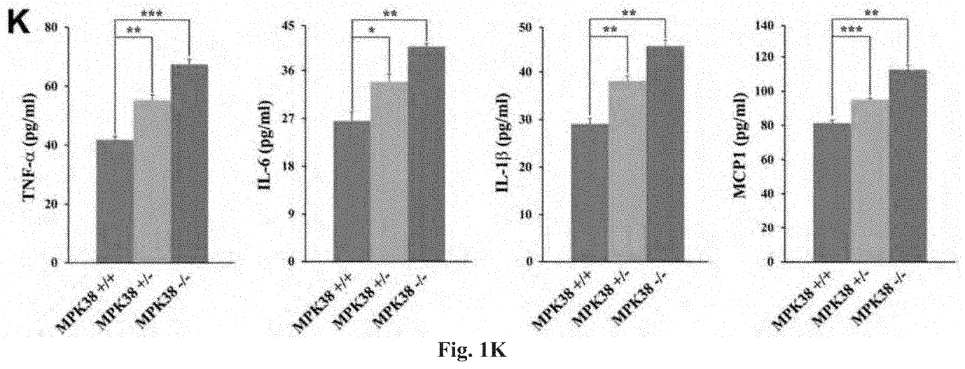

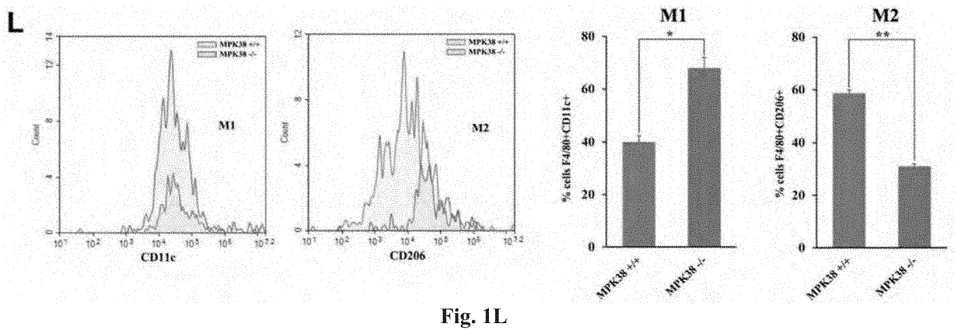

The result showed that, compared to normal mice, MPK38-knockout (MPK38^{-/-}) mice were observed to be obese using the naked eye. This phenomenon was observed only in certain male mice, but not in female mice. In addition, MPK38^{-/-} male mice were found to have higher mass of liver, spleen and epididymal fat than normal wild-type mice (WT) of the same age and gender (FIGS. 1A and 1B). When fed a standard diet, MPK38^{-/-} and WT male mice exhibited similar body mass for about 4 months, but starting at about 6 months of age, MPK38^{-/-} male mice were obese with no significant difference in feeding (FIG. 1C). Heterozygous MPK38^{+/-} male mice exhibited a moderate increase in body mass (FIG. 1C, top).

Based on these results, the present inventors identified that MPK38 deficiency induces obesity, and in particular, that obesity induced by MPK38-deficiency depends on age, gender and gene dose.

The result of glucose and insulin resistance analysis showed that MPK38^{-/-} male mice had glucose resistance and were less sensitive to insulin (FIG. 1D), and exhibited much higher blood glucose and insulin when fasted than when fed (FIG. 1E). Consistent with this, absorption of 2-deoxy-glucose stimulated with insulin was significantly lower in white adipose tissue (WAT) and muscle in MPK38^{-/-} male mice than in the normal group (FIG. 1F, left). In addition, MPK38 deficiency has been shown to decrease the activation of the insulin receptor substrate (IRS)-phosphatidylinositol 3-kinase (PI3K) pathway and reduce the expression of type 4 (GLUT4) and type 1 (GLUT1) glucose transporters in WAT and soleus muscle (FIG. 1F, right). In addition, the expression of mRNA encoding gluconeogenic proteins including G6PC (catalytic subunit of glucose 6-phosphatase), PCK1 (phosphoenolpyruvate carboxykinase-1) and PGC1-α (peroxisome proliferator-activated receptor gamma coactivator 1-alpha) was remarkably increased in MPK38^{-/-} male mice compared to a normal mouse group (FIG. 1G, top). These results showing hyperglycemia in MPK38^{-/-} male mice (FIG. 1G, bottom) mean that MPK38 is capable of inhibiting hepatic glucose production.

In addition, abnormal lipid accumulation in the liver (FIG. 2B) and levels of free fatty acids (FFA), triglycerides, total cholesterol, high-density lipoprotein-cholesterol (HDL-C) and low-density lipoprotein-cholesterol (LDL-C) in the blood (FIG. 1H) were increased in MPK38^{-/-} male mice. In addition, rapid fat biosynthesis (FIG. 1I, left), and the expression of mRNA encoding lipogenic proteins including fatty acid synthase (FAS), sterol CoA desaturase 1 (SCD1), and sterol regulatory element-binding transcription factor 1c (SREBP1c) were found to be higher in MPK38^{-/-} male mice than in the normal group (FIG. 1I, right). In addition, MPK38^{-/-} male mice showed high serum activities of AST (activities of aspartate aminotransferase) and ALT (alanine aminotransferase), which represent the fatty liver phenotype (FIG. 2C). Consistent with these results, mean adipocyte size was also found to be higher in MPK38^{-/-} male mice than in heterozygous mice (MPK38^{+/-}) or normal mice (FIG. 1J), and mRNA expression of adipogenic regulators including C/EBPα (CCAAT-enhancer-binding protein α), PPARγ (peroxisome proliferator-activated receptor gamma), and FABP4 (fatty acid binding protein 4) was increased in MPK38^{-/-} male mice (FIG. 2D).

In addition, it was found through flow cytometry that higher levels of inflammatory reactions were induced in MPK38^{-/-} male mice; specifically, the amounts of proinflammatory cytokines including tumor necrosis factor-α (TNF-α) and interleukin-6 (IL-6), monocyte chemoattractant protein 1 (MCP1) and a number of M1 macrophages were increased (FIGS. 1K and 1L).

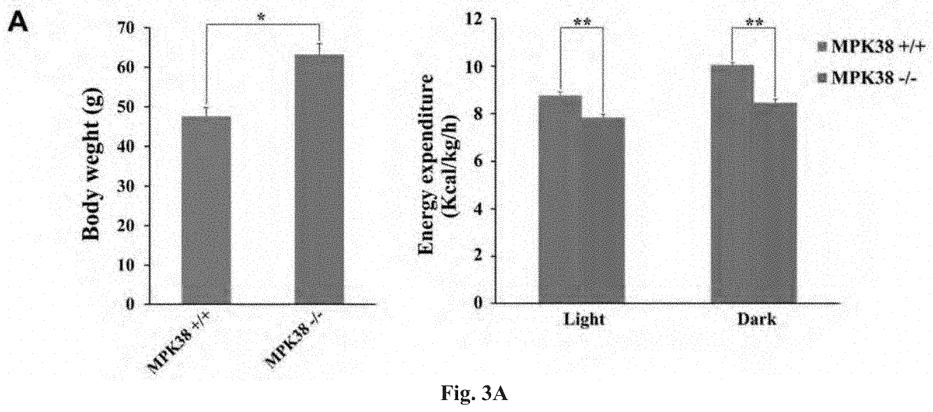

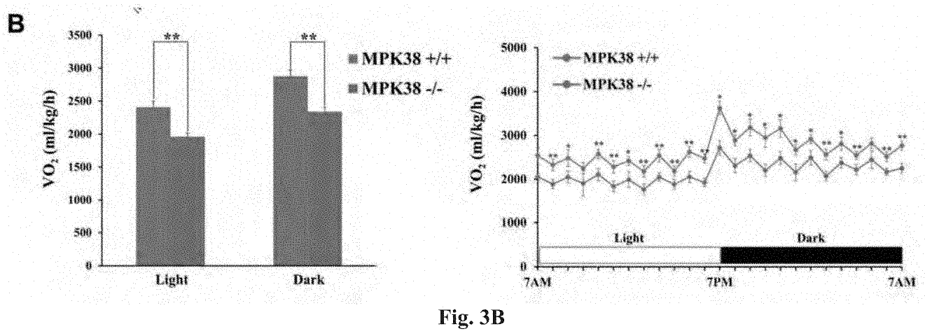

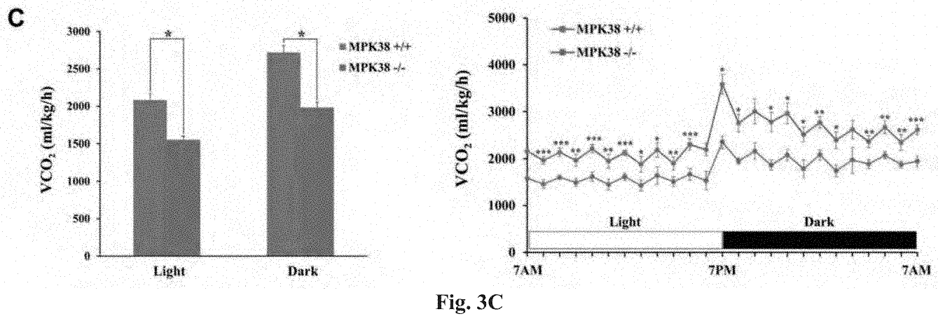

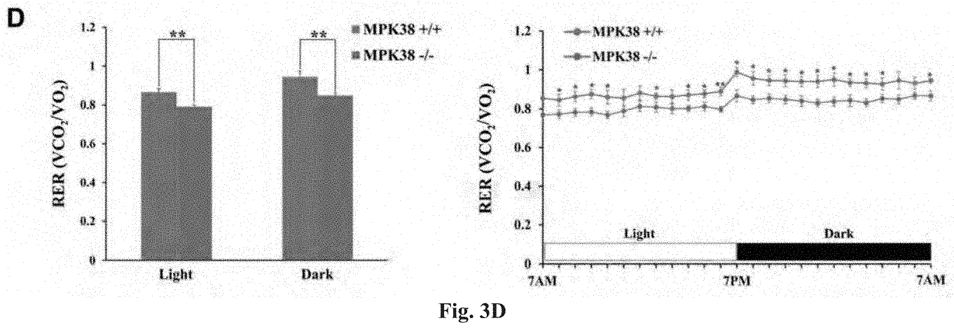

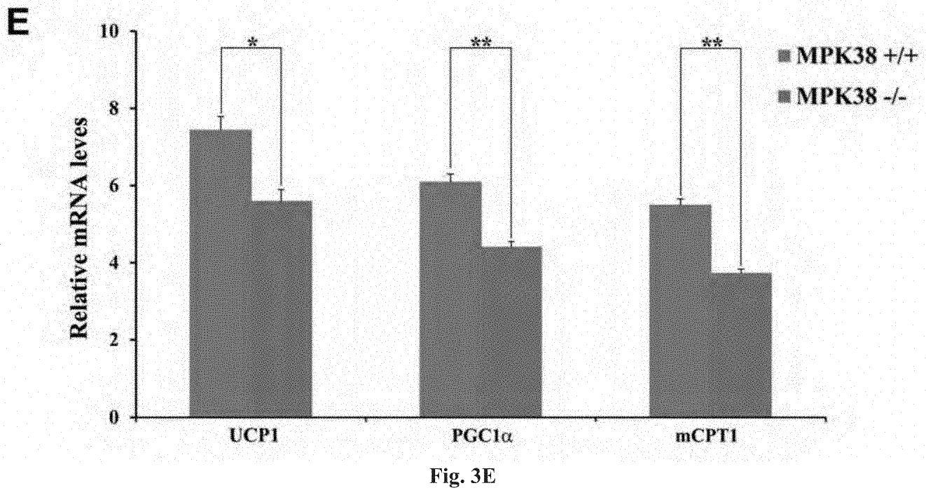

The results of analysis of the effects of MPK38 deficiency on energy metabolism are shown in FIG. 3, and show that MPK38^{-/-} male mice exhibit lower energy consumption, 24-hour O₂ consumption, CO₂ production, and respiratory exchange rate (RER) compared to the normal group. In addition, the expression of uncoupling protein 1 (UCP1), PGC1α, and muscle-type carnitine palmitoyltransferase 1 (mCPT1), which are greatly involved in thermogenesis, was found to be lower than that of the normal group.

Overall, based on these results, the present inventors found that MPK38 deficiency may result in abnormalities in obesity and lipid metabolisms specifically for middle-aged men, leading to obesity and metabolic diseases, and that MPK38 acts to prevent or treat obesity and metabolic diseases specifically for middle-aged men.

### Example 2

Confirmation of Worsening of Obesity and Metabolic Disorders in Middle-Aged Men Due to MPK38 Deficiency

To determine whether or not MPK38 has protective activity against metabolic disorders caused by a high-fat diet (HFD), as 7-month-old male mice, normal mice (WT), MPK38^{+/-} male mice and MPK38^{-/-} male mice were fed a standard diet and a high-fat diet, and then lipid-metabolism-related changes were analyzed.

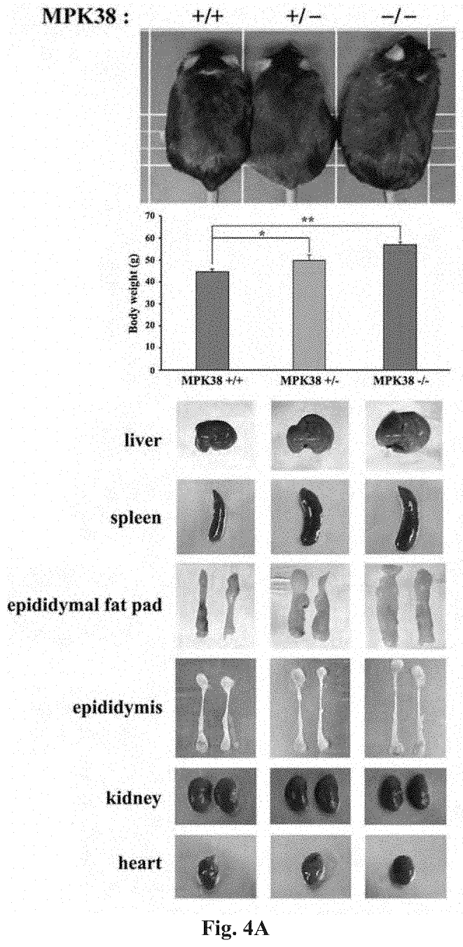

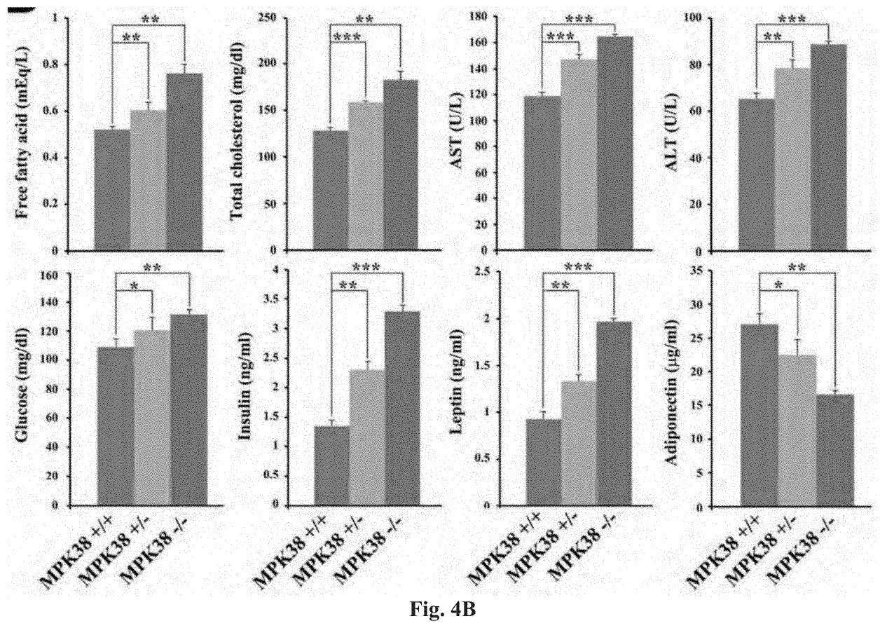

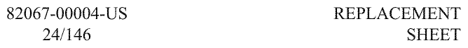

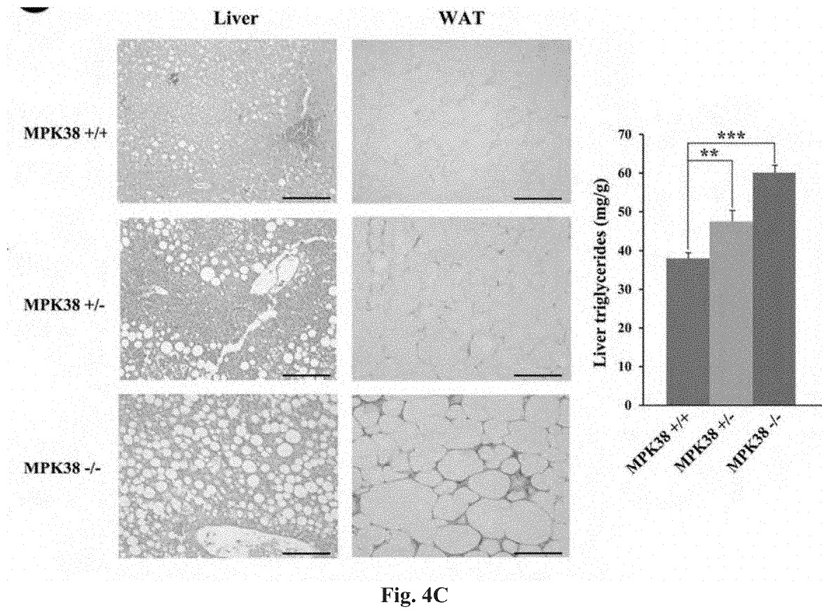

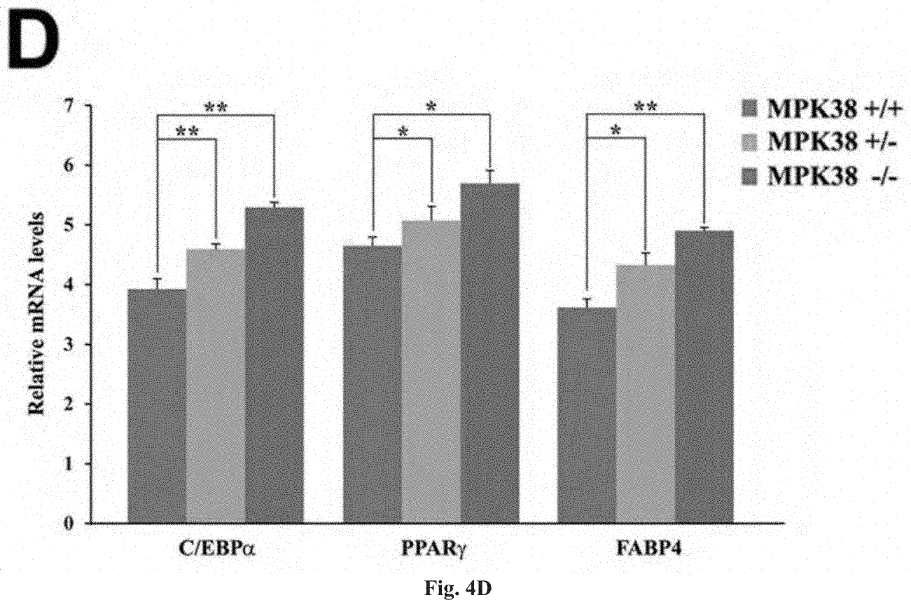

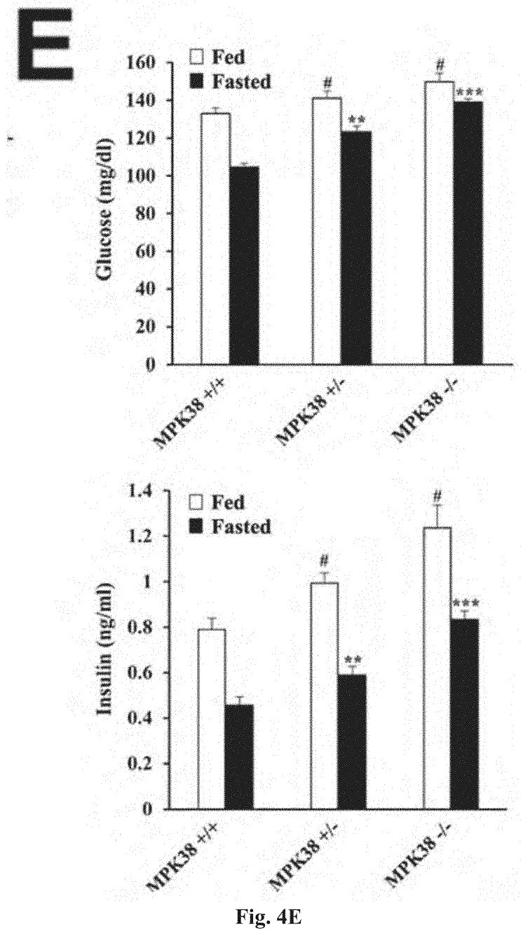

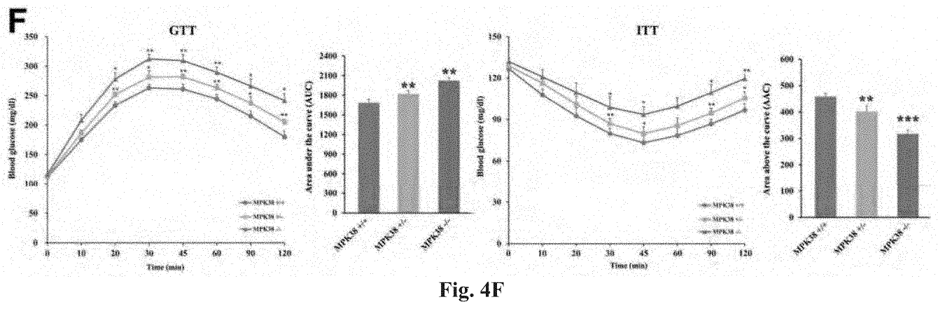

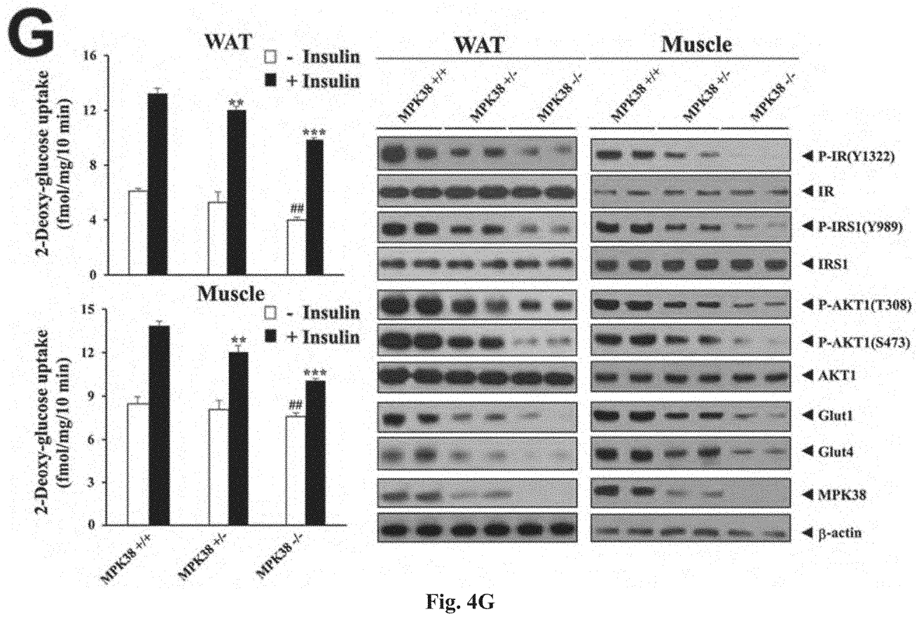

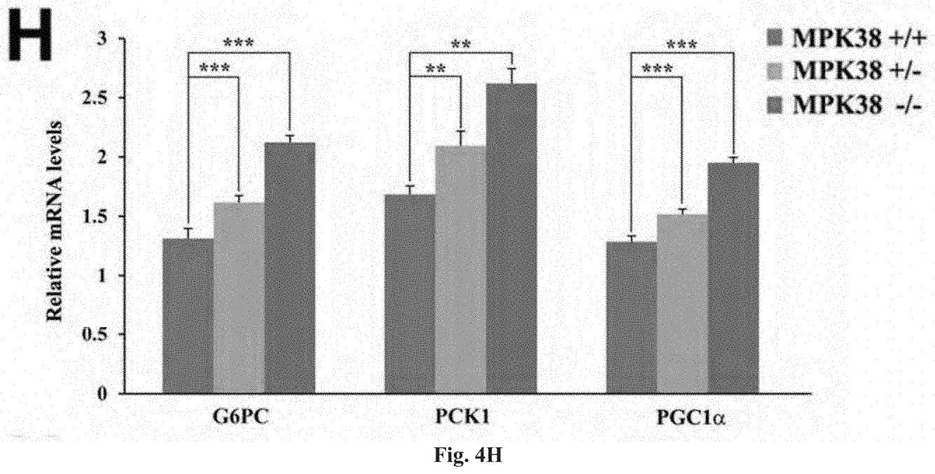

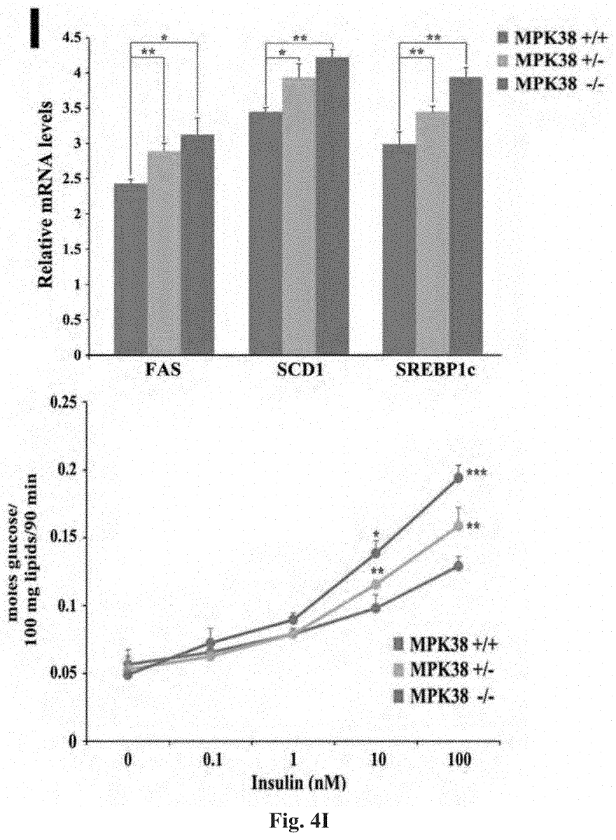

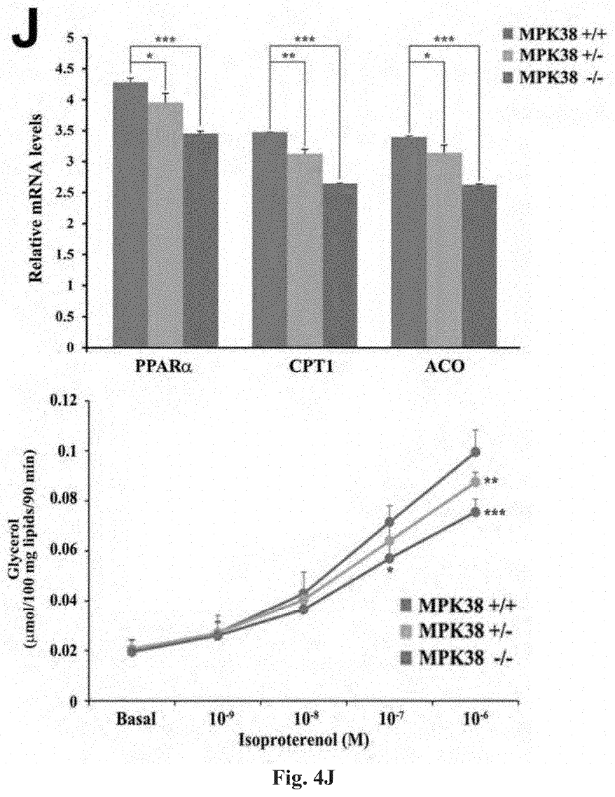

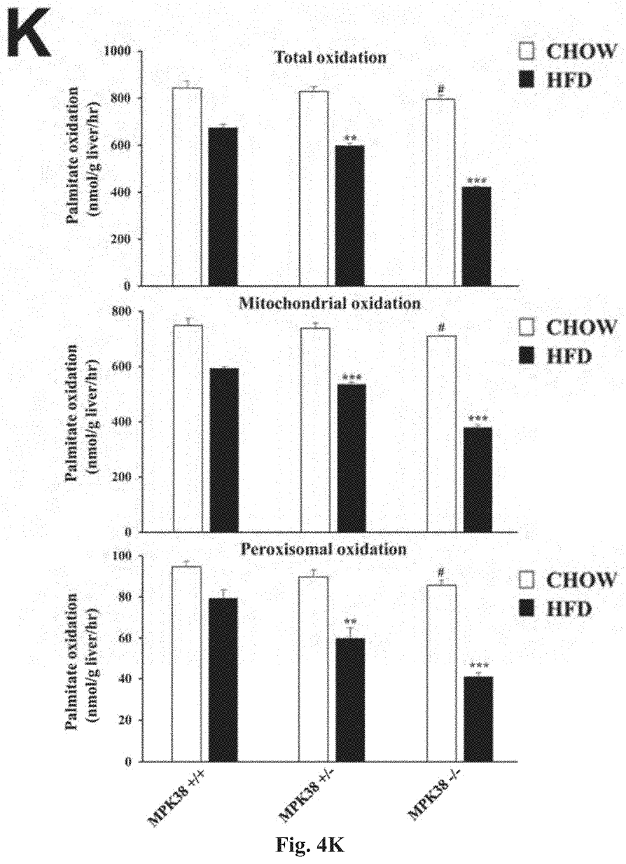

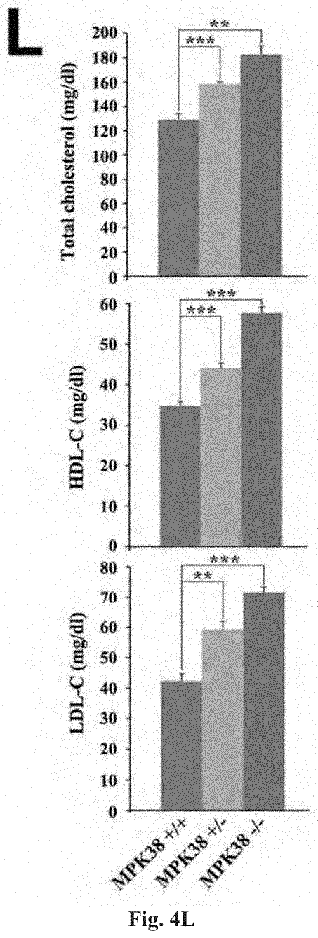

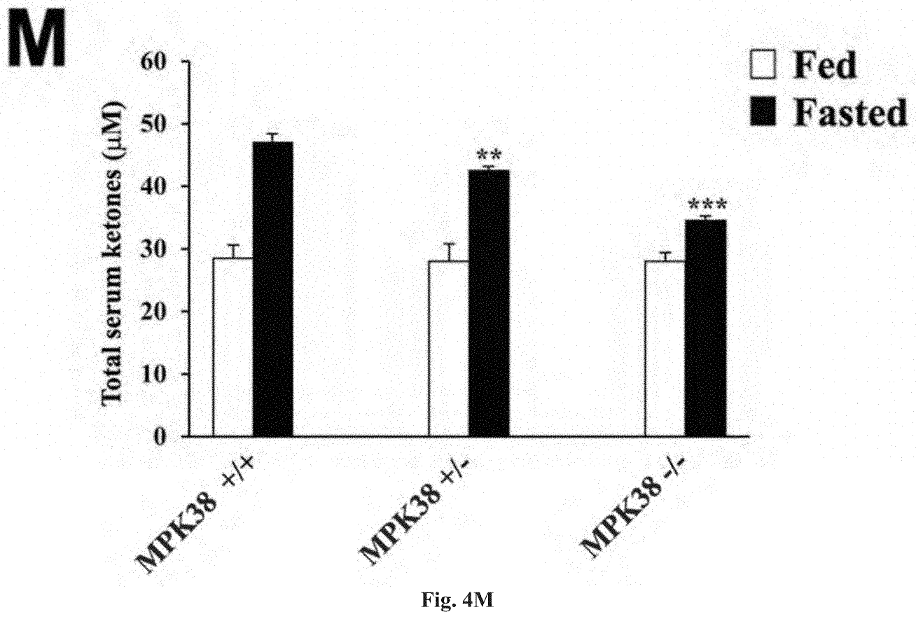

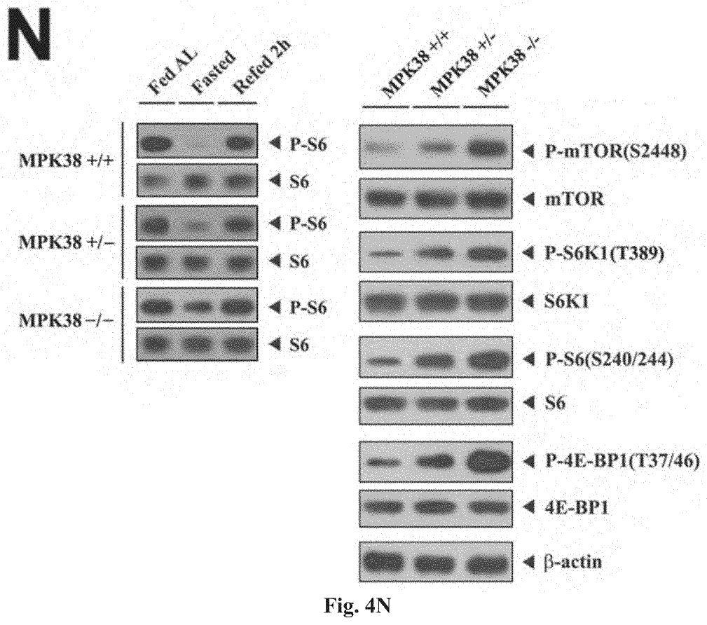

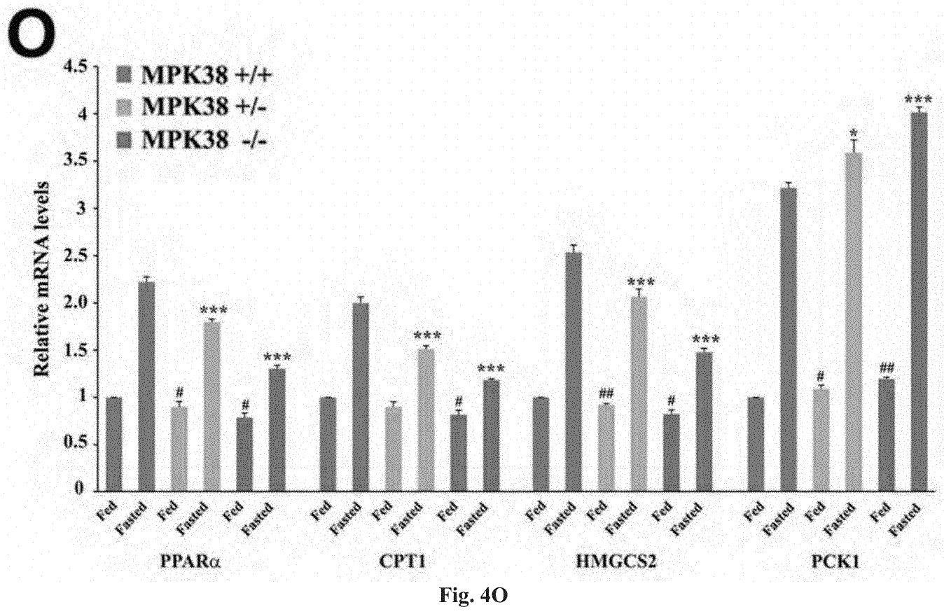

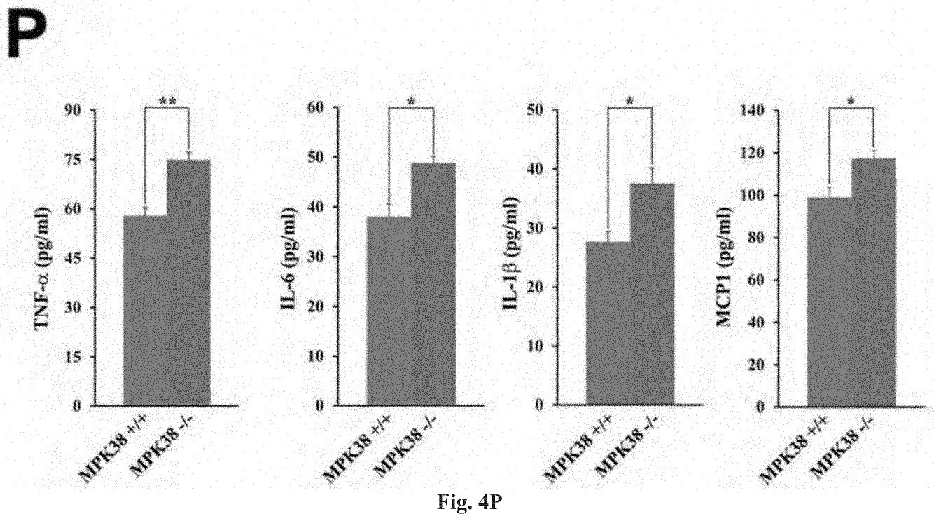

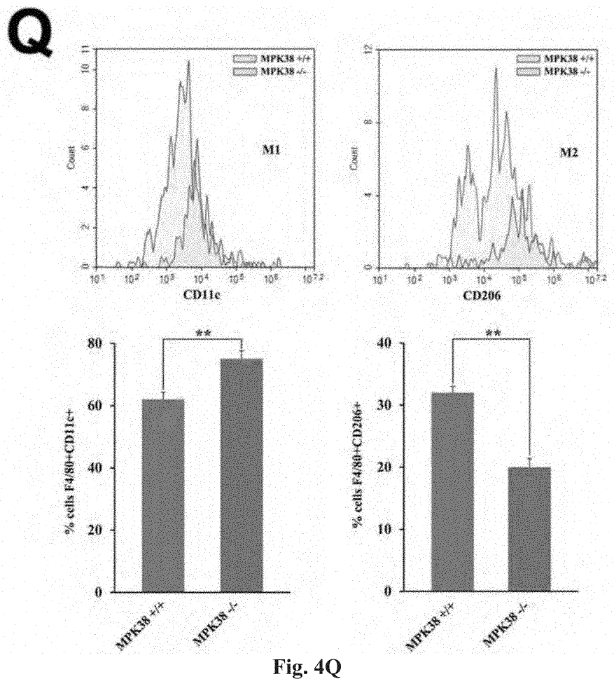

As a result, as can be seen from FIG. 4, compared to normal mice, MPK38^{-/-} male mice became more obese, did not properly regulate glucose or lipid metabolism, and exhibited higher levels of inflammation under HFD supply conditions.

Specifically, high-fat-diet-fed MPK38^{-/-} male mice exhibited the highest levels of blood glucose and insulin (FIG. 4E), and exhibited increased glucose intolerance and insulin resistance (FIG. 4F). Consistent with these results, it was found that the absorption of 2-deoxy-glucose due to insulin stimulation was significantly lowered in the WAT and muscles of high-fat-diet-fed MPK38^{-/-} male mice (FIG. 4G). In addition, the mRNA expression of gluconeogenic proteins including G6PC, PCK1 and PGC1α was further increased in high-fat-diet-fed MPK38^{-/-} male mice compared to high-fat-diet-fed normal male mice (FIGS. 4B and 4H).

Compared to high-fat-diet-fed normal mice, the high-fat-diet-fed MPK38^{-/-} male mice exhibited higher proportions of lipogenesis and higher expression levels of mRNA encoding lipogenic proteins including FAS, SCD1 and SREBP1c (FIG. 4I), increased expression of C/EBPα, PPARγ, and FABP4, which are key adipogenic regulators (FIG. 4D), and increased inflammatory response (FIGS. 4P and 4Q).

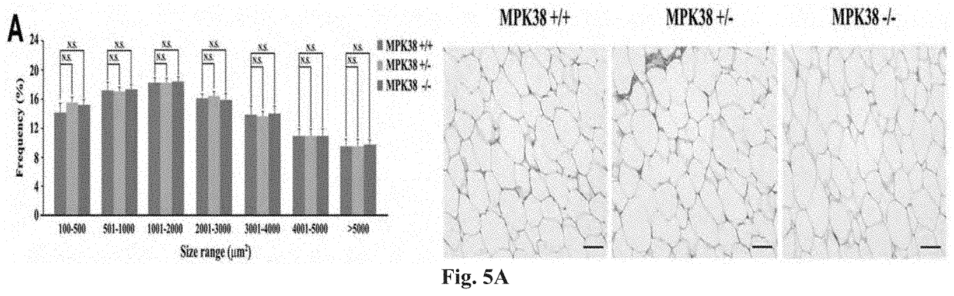

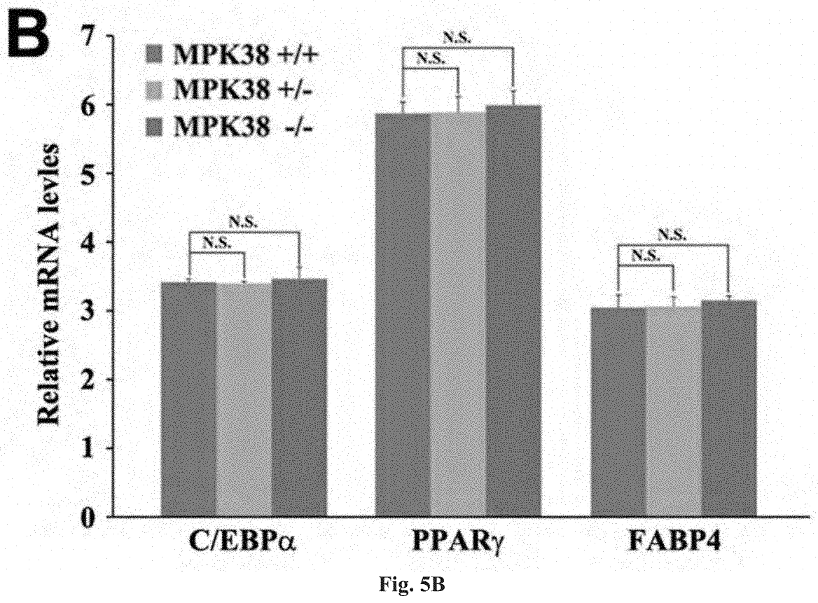

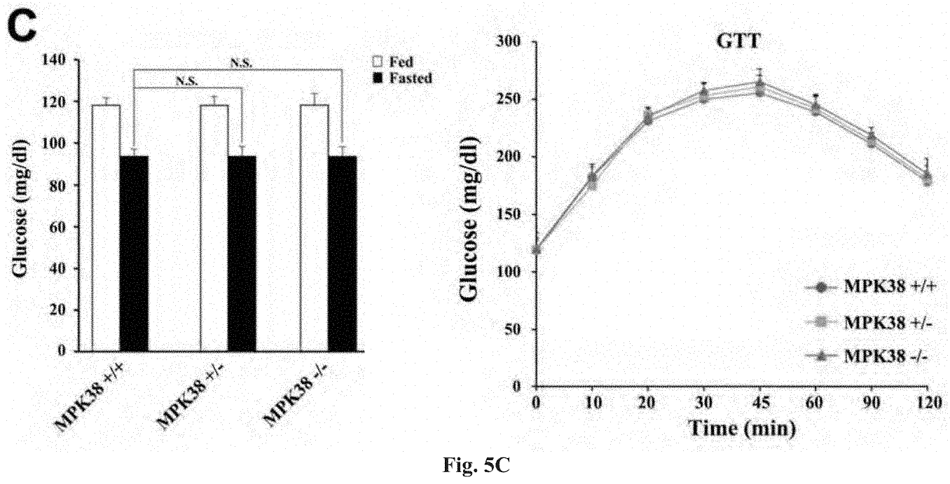

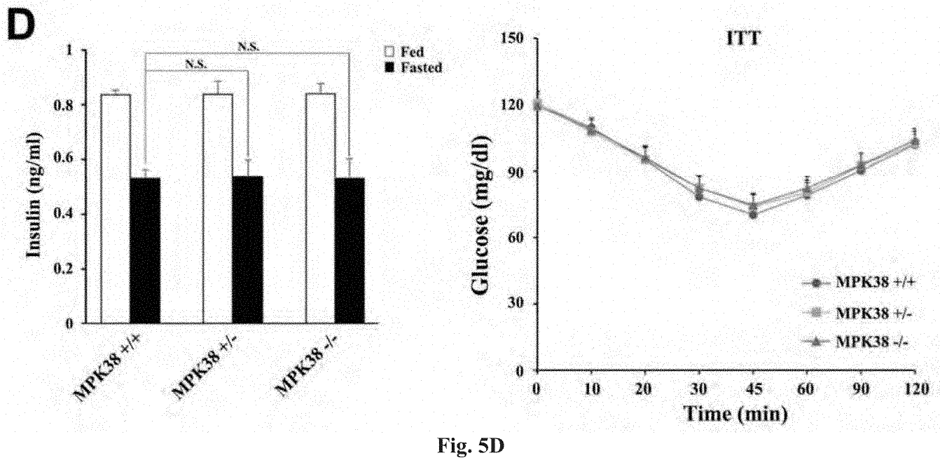

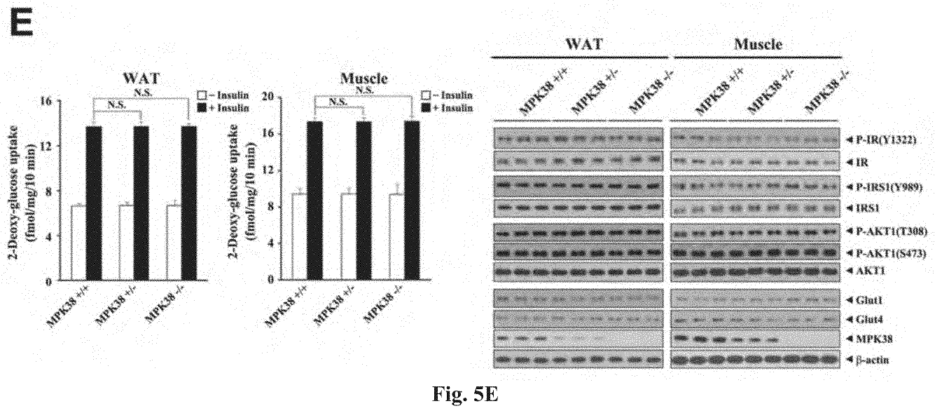

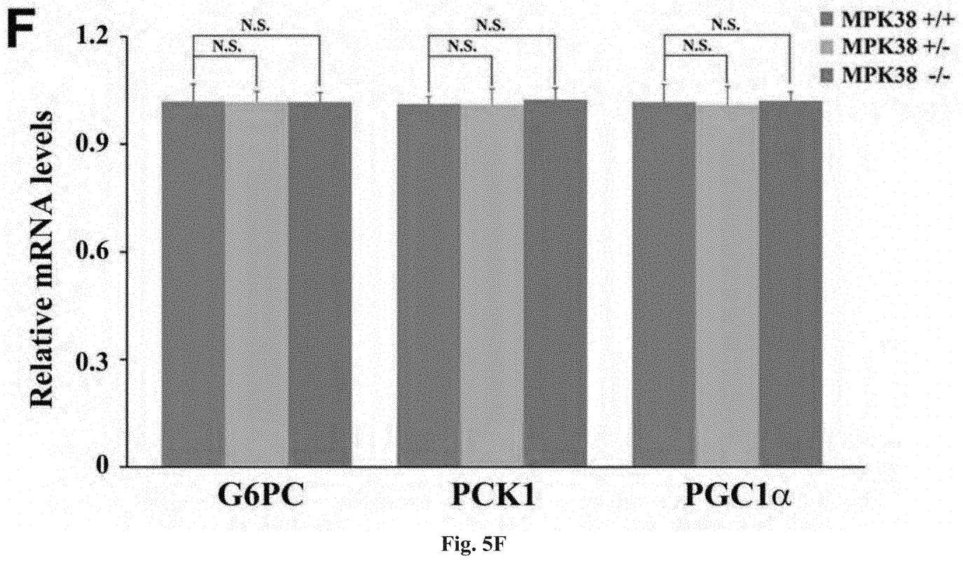

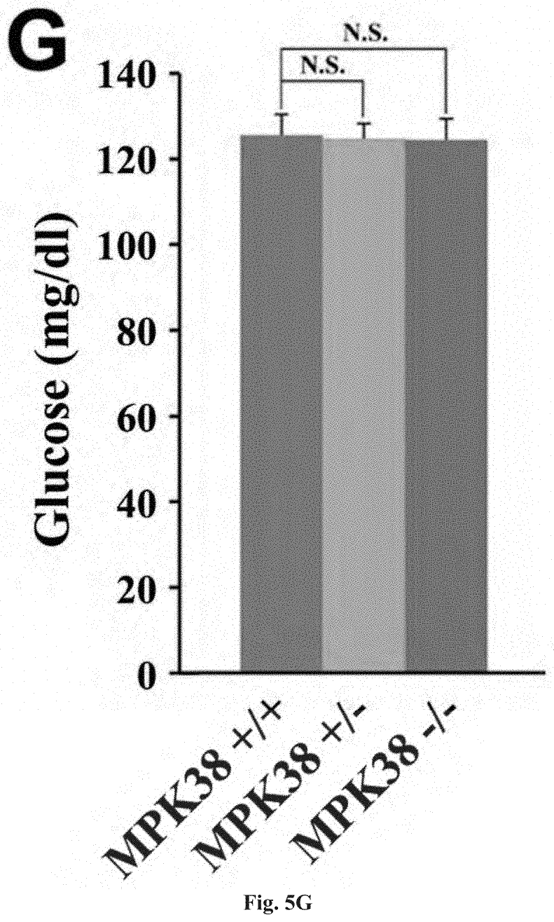

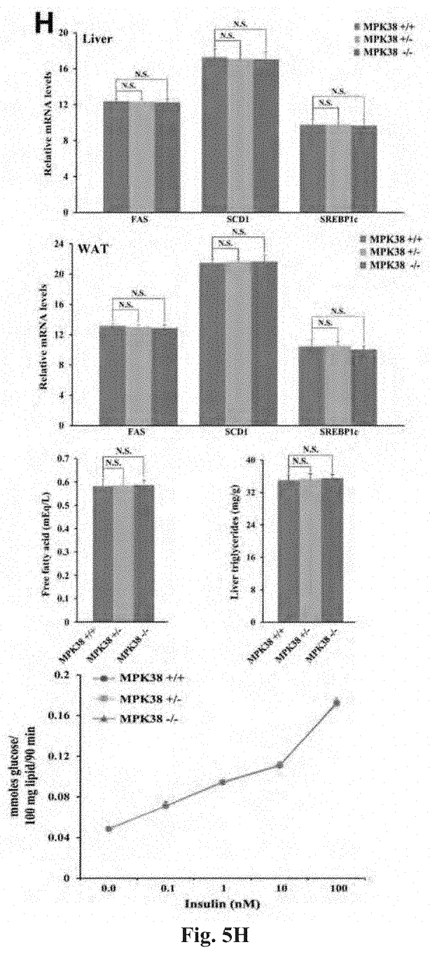

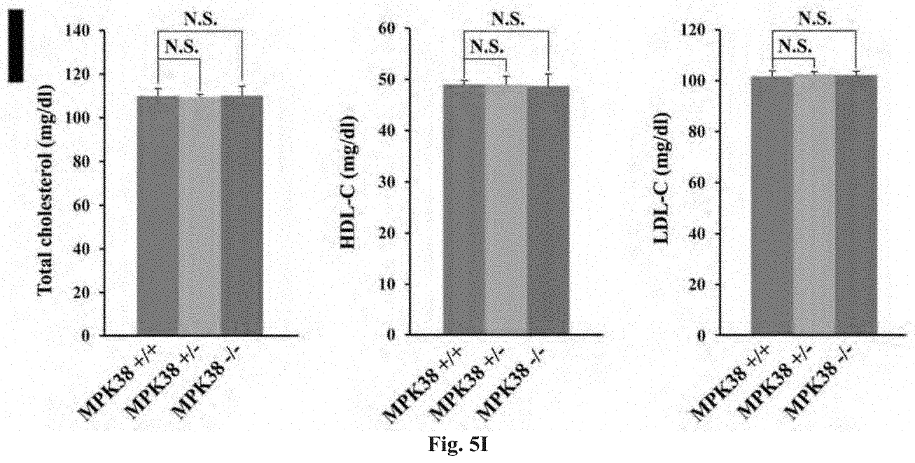

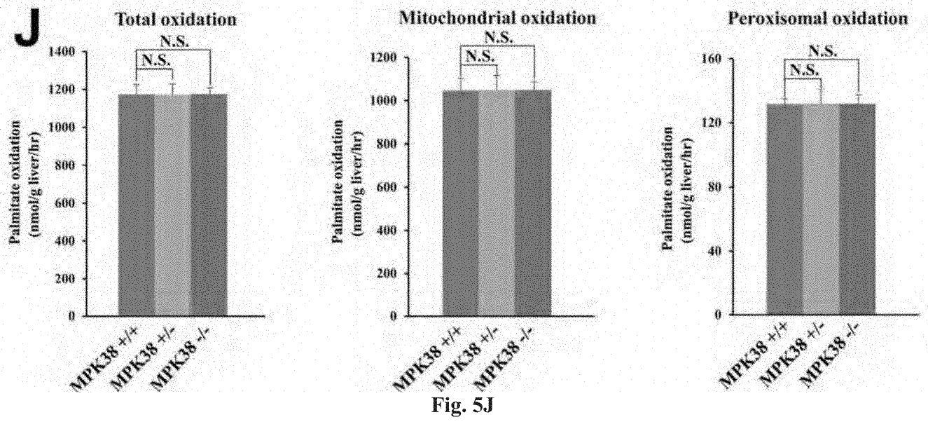

Meanwhile, the abnormal symptoms induced by MPK38 deficiency observed in high-fat-diet-fed male mice were not observed in female mice (see FIG. 5).

This indicates that the effects of MPK38 on obesity and metabolic disorders do not occur in women, but occur specifically for middle-aged men, and that the availability of MPK38 as a therapeutic agent for treating obesity and metabolic disorders specific for middle-aged men can be predicted.

### Example 3

Confirmation of Efficacy of Amelioration in Obesity and Metabolic Disorders Specific to Middle-Aged Men by Induction of MPK38 Expression

Whether or not MPK38 activates ASK1/TGF-β/p53 signaling was determined.

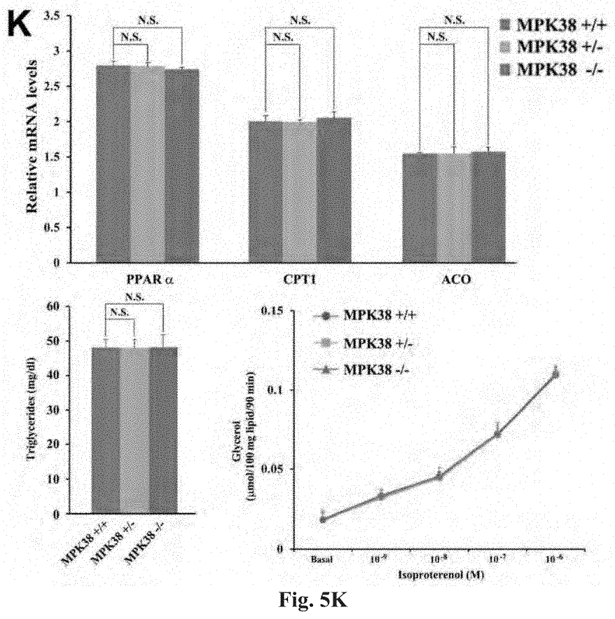

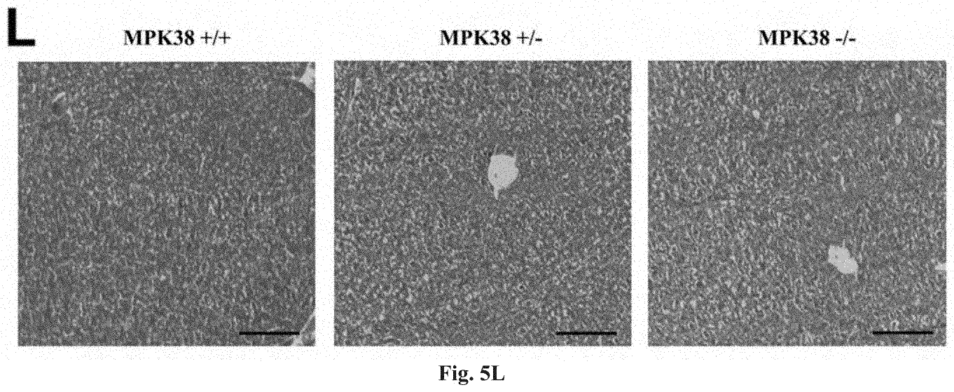

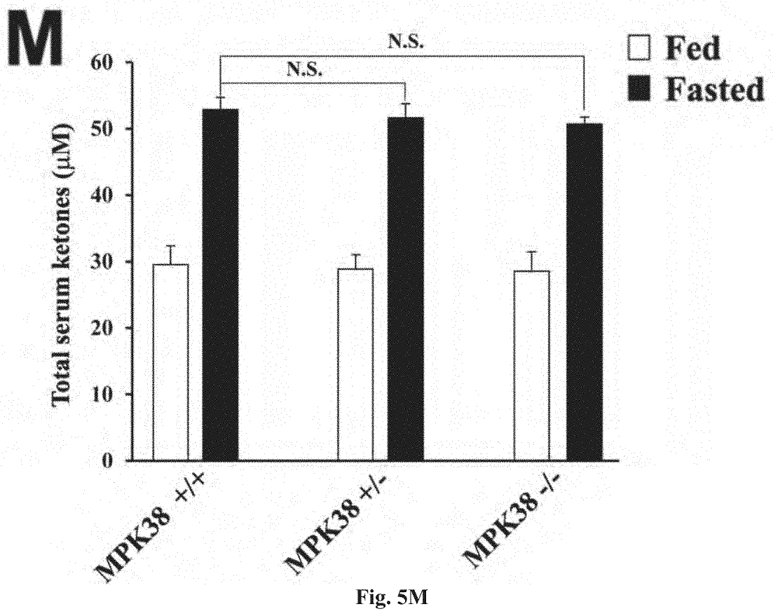

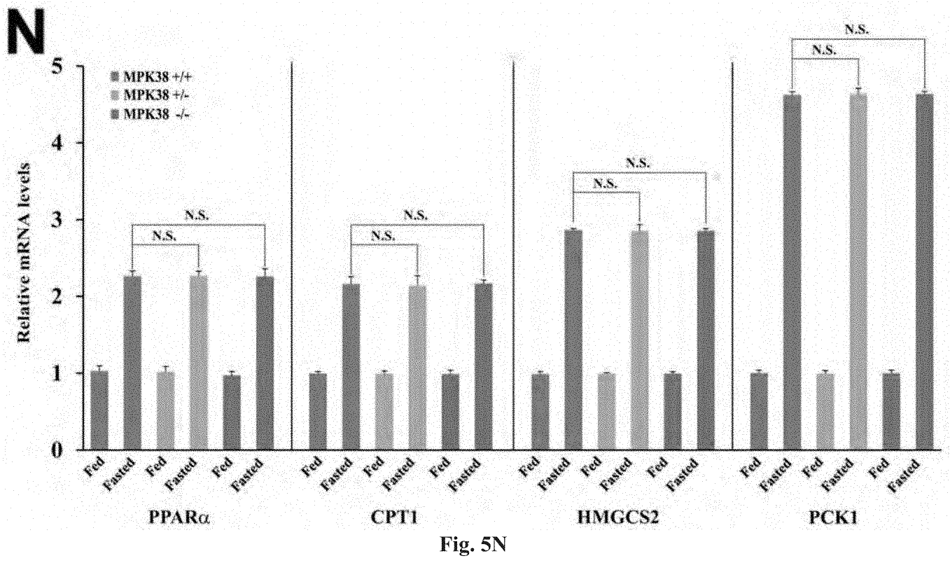

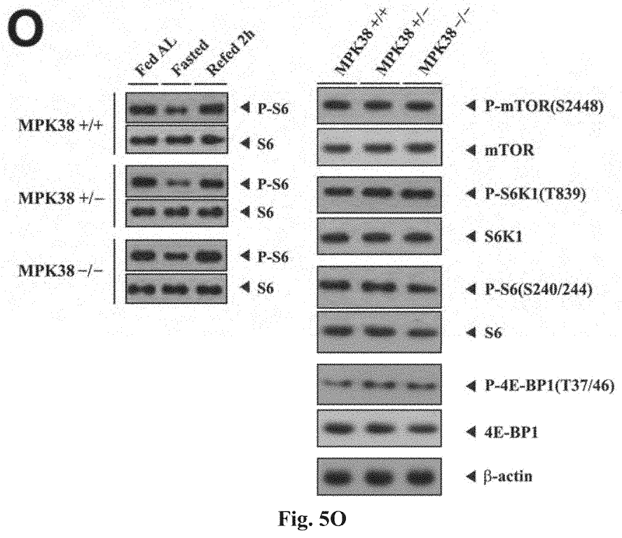

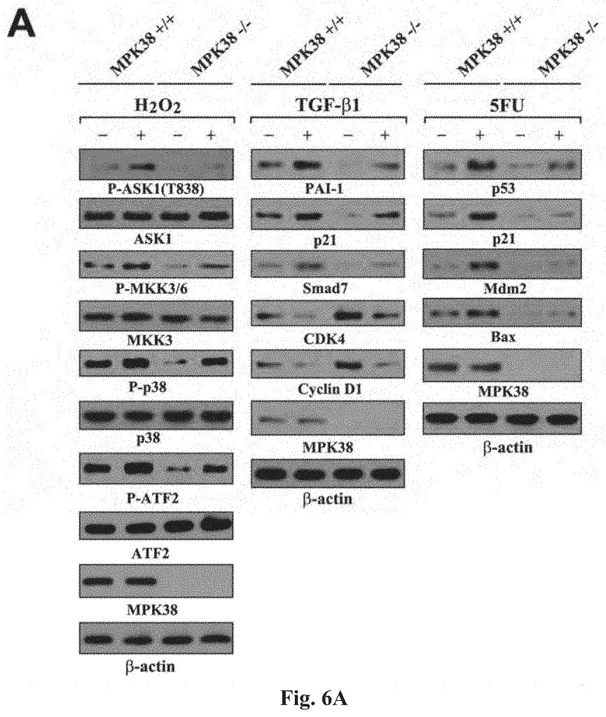

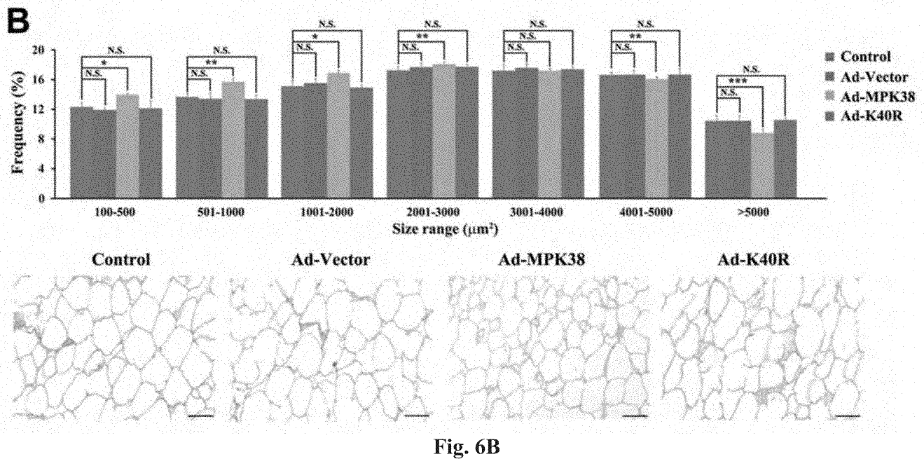

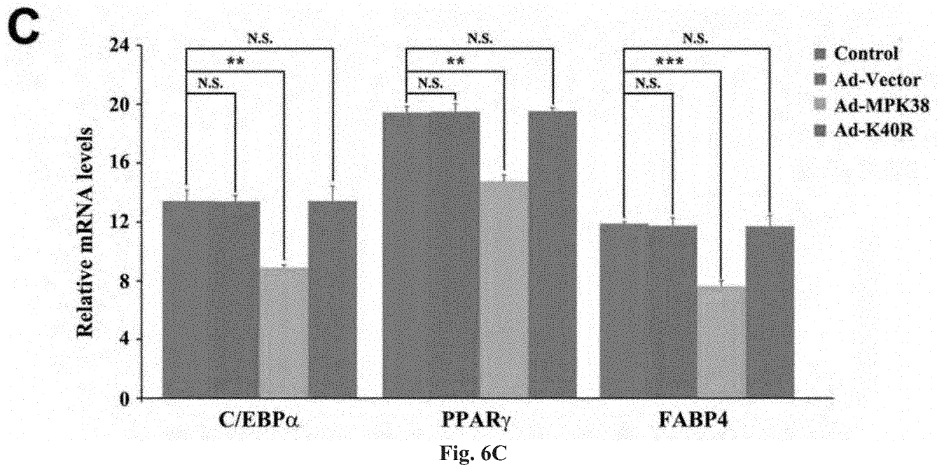

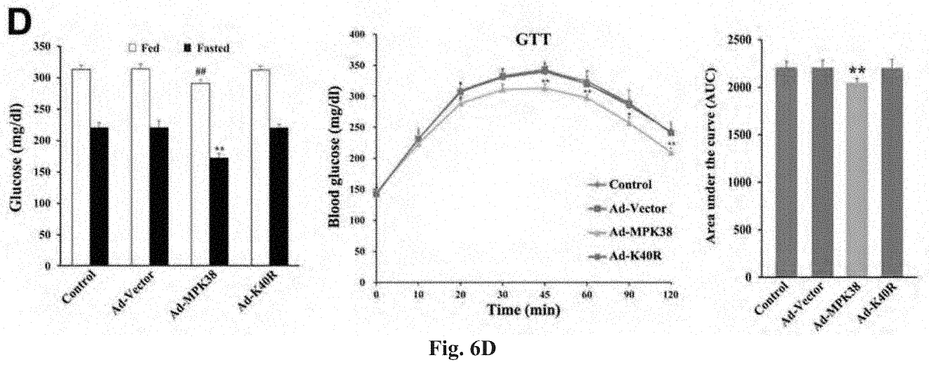

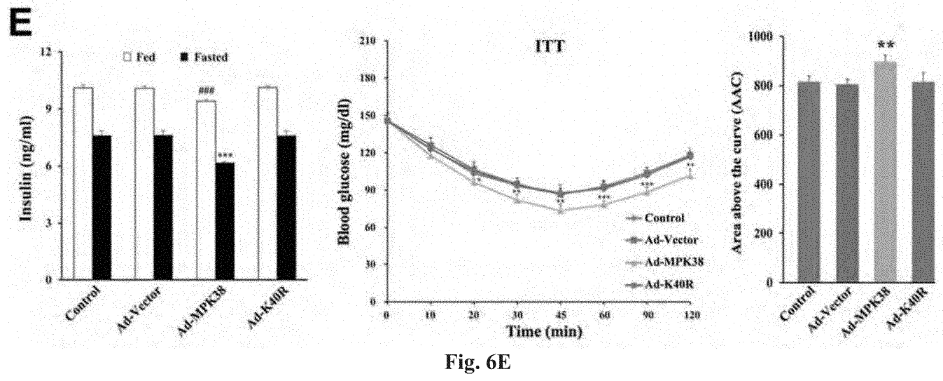

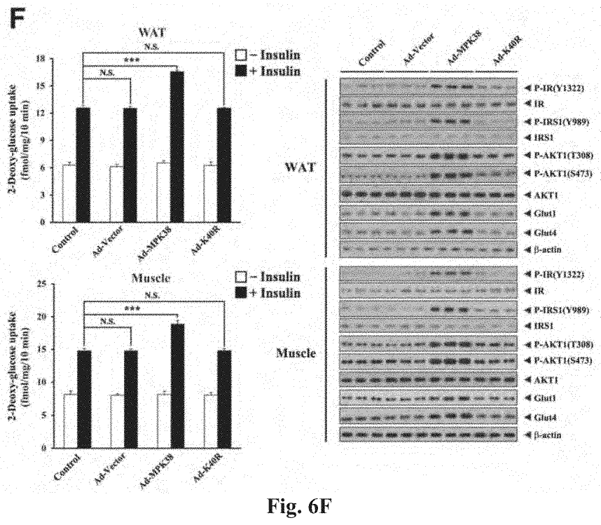

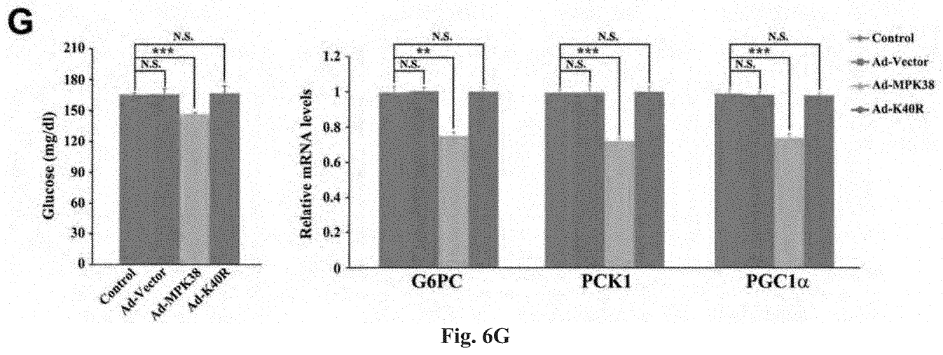

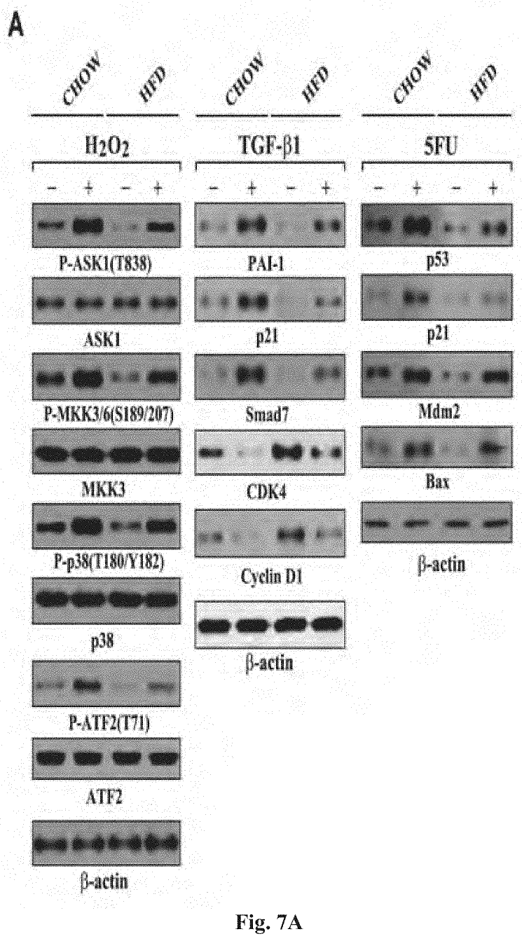

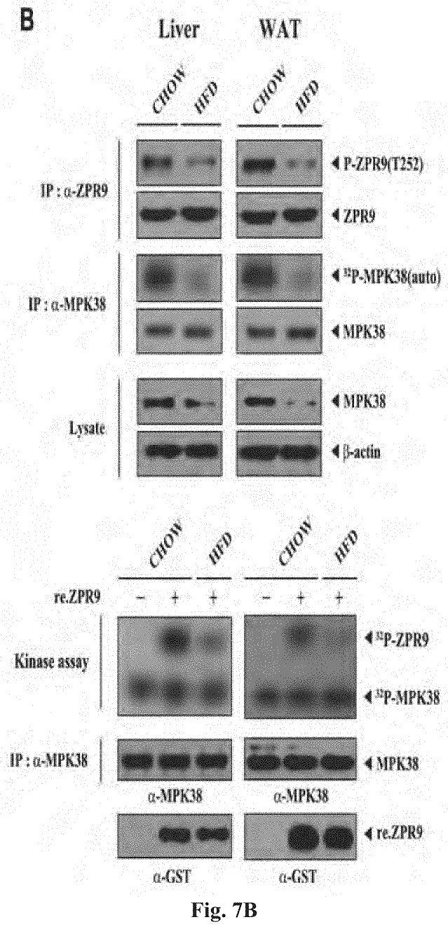

The result showed that the activation of MPK38-dependent ASK1/TGF-β/p53 signaling was decreased in 7-month-old MPK38^{-/-} male mice compared to normal mice of the same age and gender (see FIG. 6). Similar thereto, ASK1/TGF-β/p53 signaling, MPK38 expression and kinase activity were decreased in obese male mice (ob/ob) and high-fat-diet-fed male mice (FIG. 7).

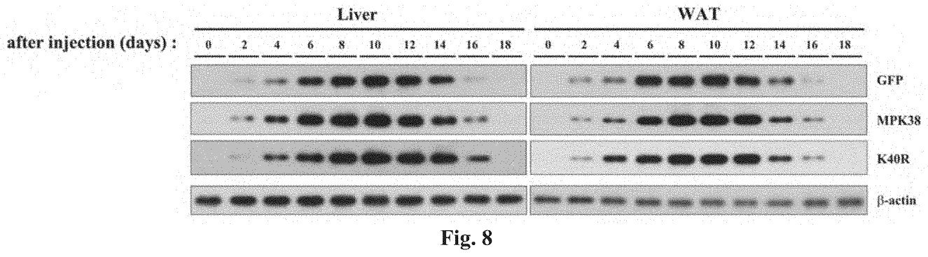

In addition, in order to analyze the gender-specific effects of MPK38 on the amelioration of obesity and related metabolic disorders, the expression of WT MPK38 (Ad-MPK38) was induced in 7-month-old middle-aged high-fat diet male mice using adenovirus. At this time, as a control group, K40R MPK38 (Ad-K40R), from which kinase activity is lost, was also introduced and expressed, and normal expression of WT MPK38 and K40R MPK38 was ascertained through Western blot (see FIG. 8).

As a result, the group introduced with WT MPK38 exhibited a decrease in the size of adipocytes (FIG. 6B), and a decrease in the expression of lipogenic proteins (C/EBPα, PPARγ, and FABP4) (FIG. 6C). These results indicate that the expression of MPK38 or induction of activity thereof can inhibit lipogenesis and can further increase insulin sensitivity.

In addition, Ad-MPK38-introduced high-fat-diet-fed male mice exhibited lower blood glucose and insulin concentrations and increased glucose tolerance and insulin sensitivity compared to high-fat diet male mice in which MPK38 expression was not induced (FIGS. 6D and 6E).

It was found that induction of MPK38 expression by introduction of Ad-MPK38 improves 2-deoxyglucose absorption in both the epididymal WAT and muscle (FIG. 6F, left), which may be explained by upregulation of the IRS-PI3K pathway and high expression of glucose transporters (FIG. 6F, right). It was found that blood glucose was decreased due to decreased expression of gluconeogenic proteins (G6PC, PCK1 and PGC1α) in the liver (FIG. 6G).

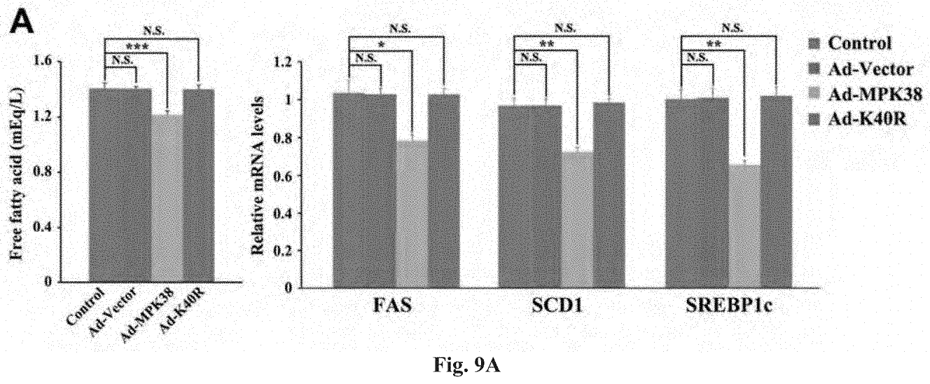

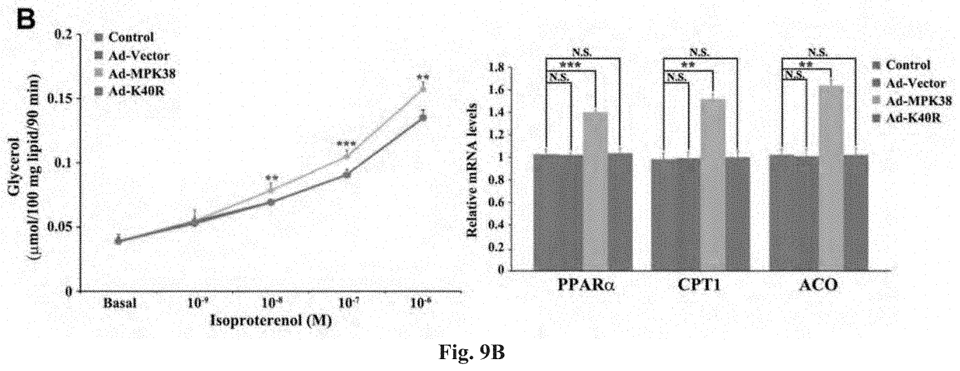

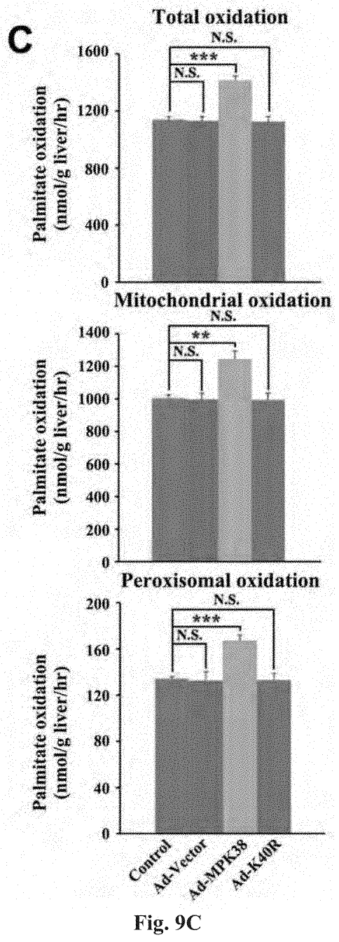

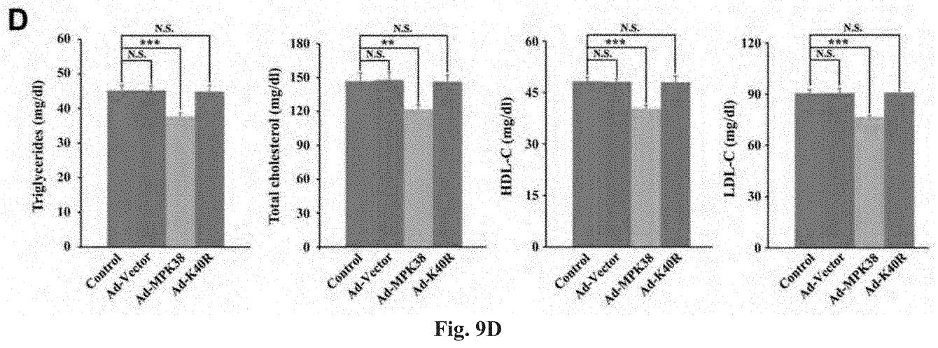

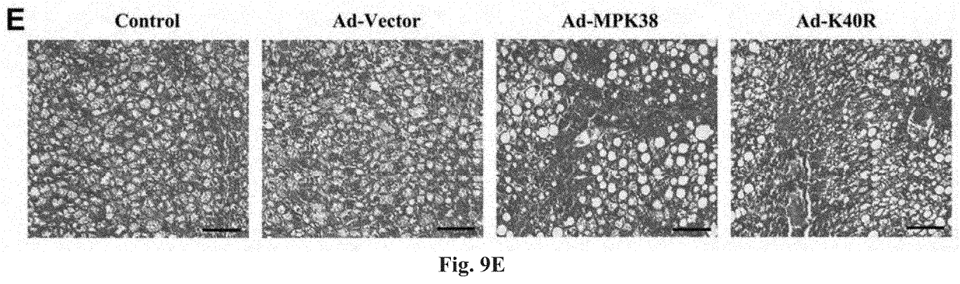

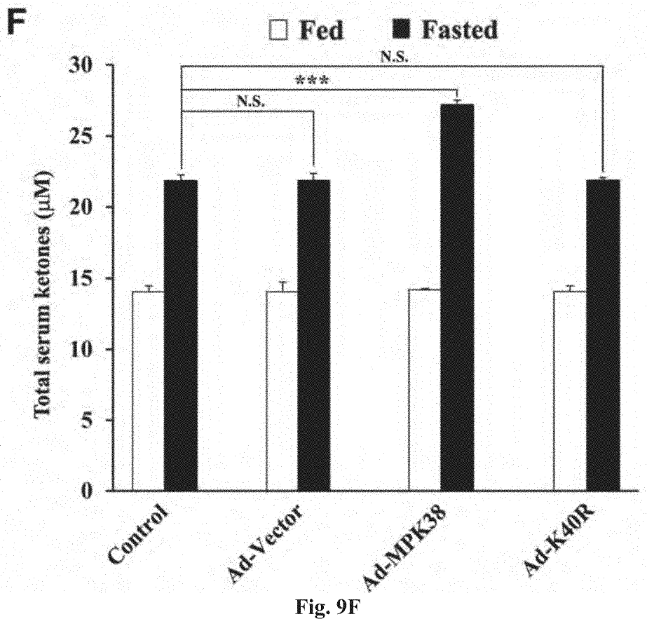

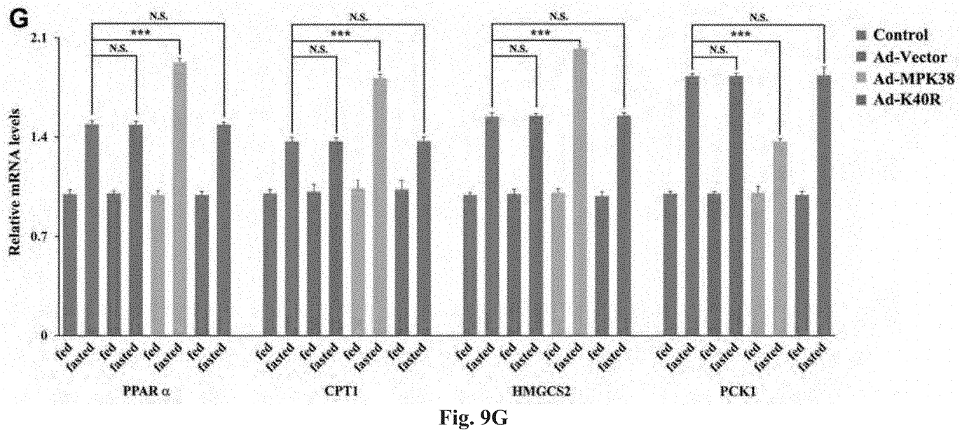

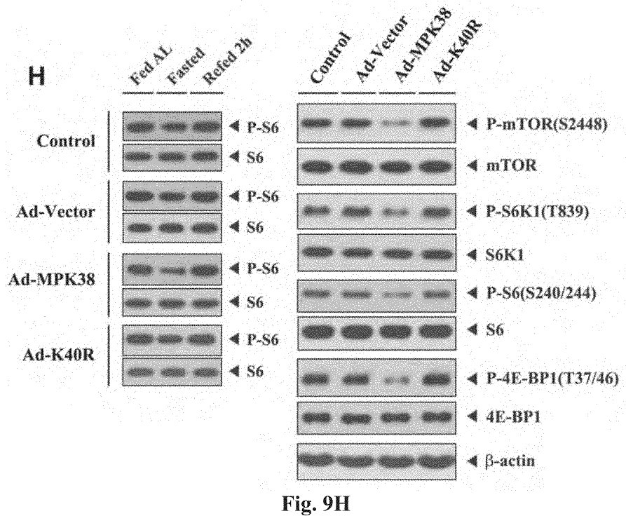

Furthermore, the induction of expression of MPK38 decreases the FFA concentration in the blood (FIG. 9A, left) and the expression of lipogenic proteins (FAS, SCD1 and SREBP1c) (FIG. 9A, right), and increases lipolysis by increased isoproterenol (FIG. 9B, left) and the expression of fatty acid-oxidizing proteins (PPARα, CPT1 and ACO) (FIG. 9B, right).

Therefore, it was found that the induction of expression of MPK38 facilitates the oxidation of fatty acids through β-oxidation of mitochondria and peroxisomes, thereby upregulating fatty acid utilization.

Next, it was found that, when Ad-MPK38 was introduced into high-fat diet male mice to induce the expression of MPK38, the levels of triglycerides, total cholesterol, HDL-C and LDL-C in the blood were lowered (FIG. 9D), and lipid accumulation in the liver was reduced (FIG. 9E). These results indicate that MPK38 has a positive effect on lipid metabolism. In addition, the induction of expression of MPK38 was found to promote the production of ketone bodies (FIG. 9F), and the expression of ketogenic genes was increased under fasting conditions (FIG. 9G), but mTORC1 signaling was decreased (FIG. 9H).

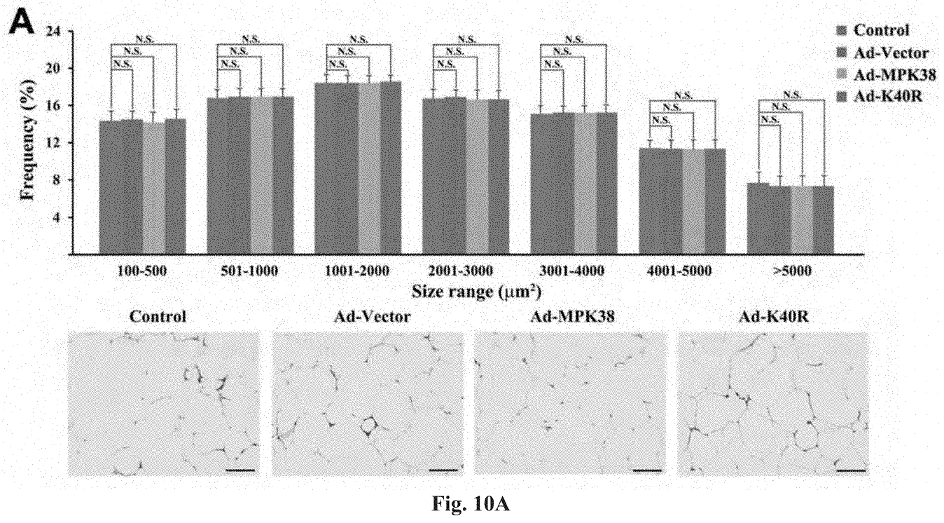

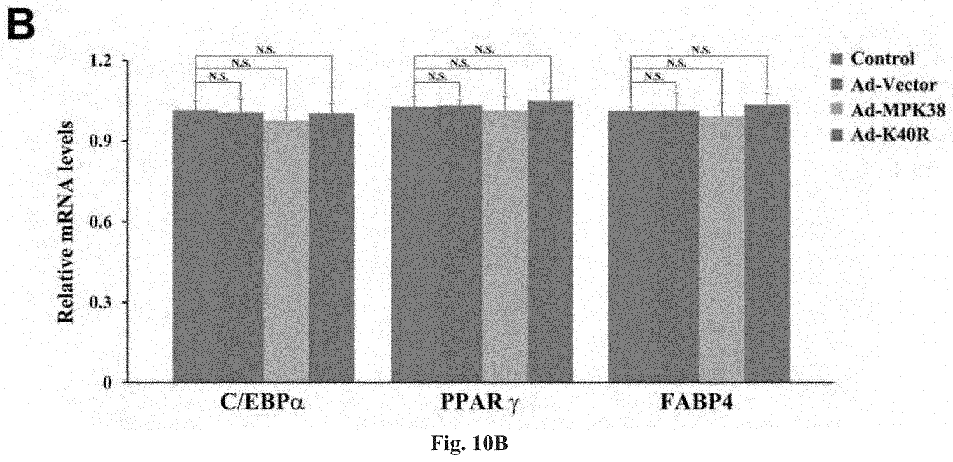

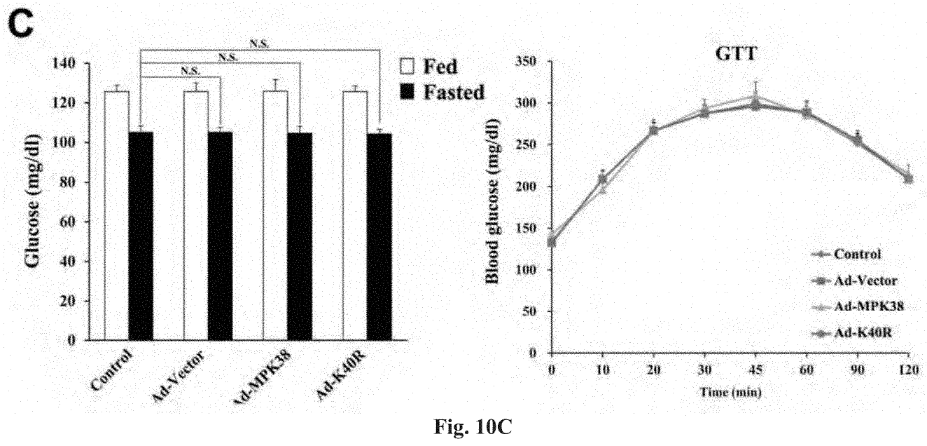

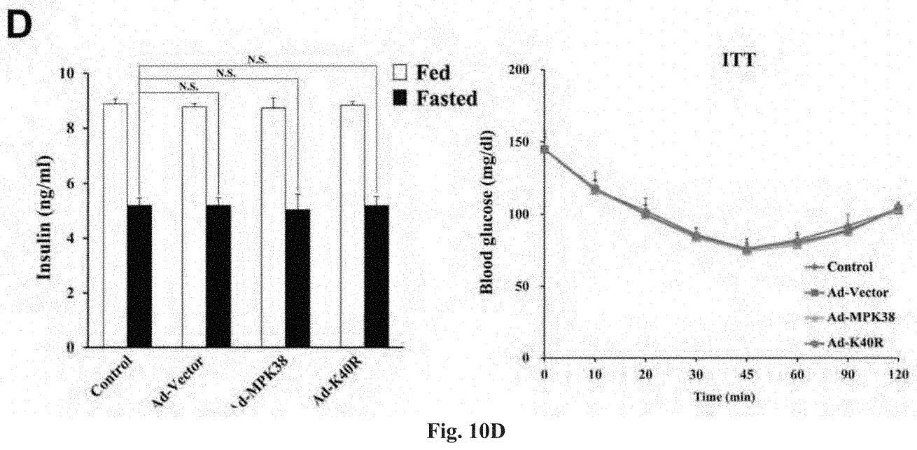

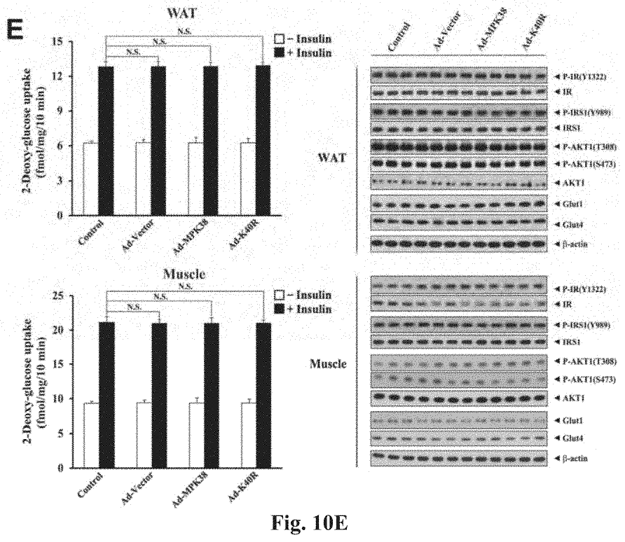

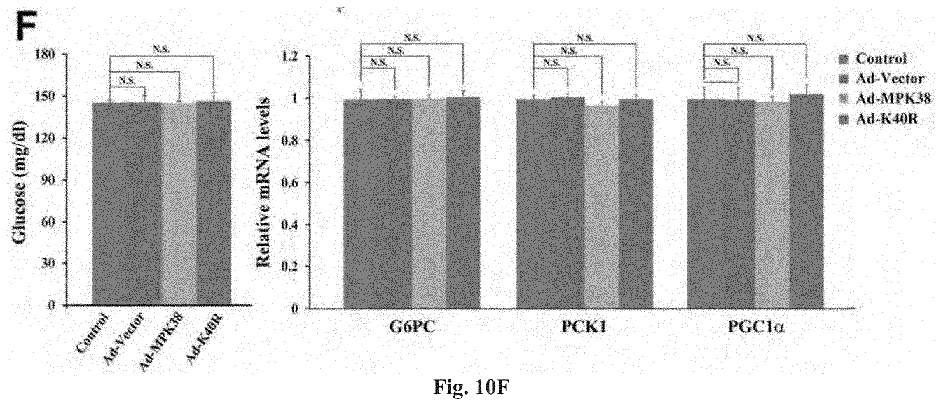

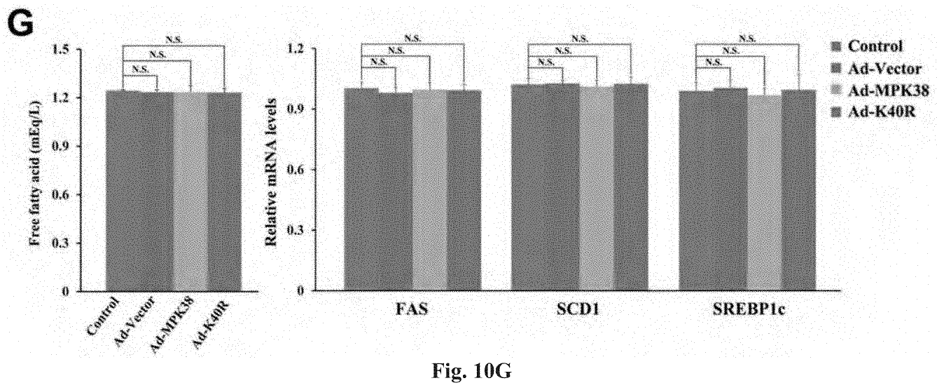

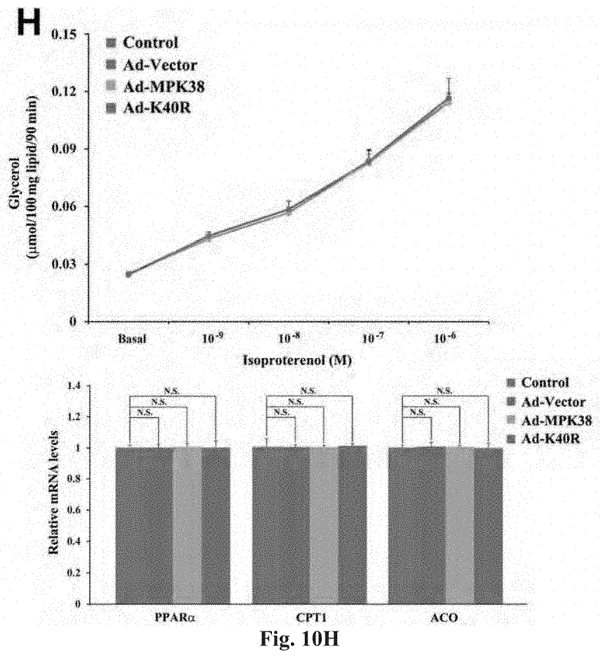

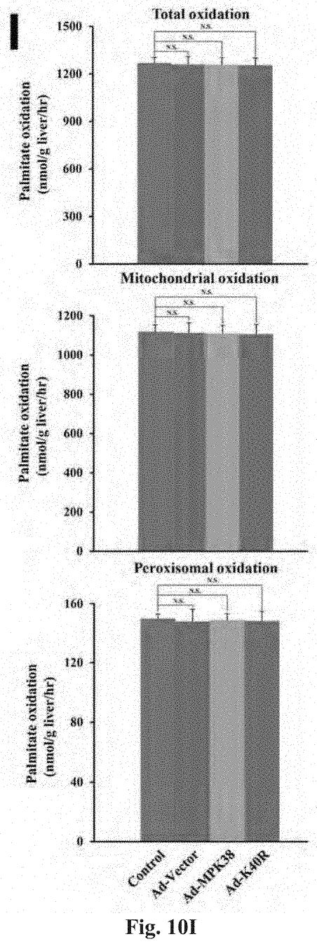

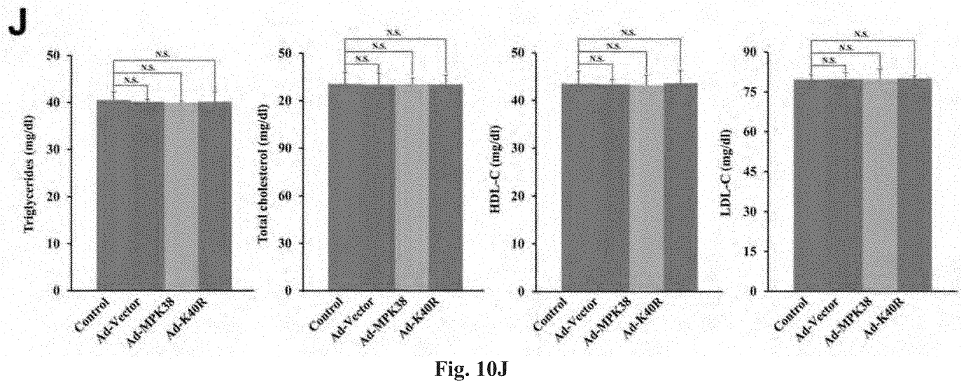

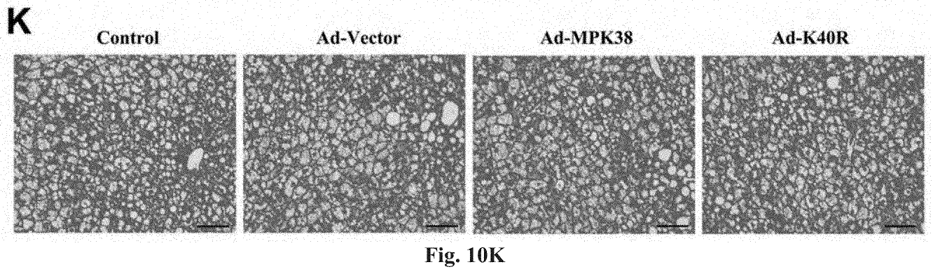

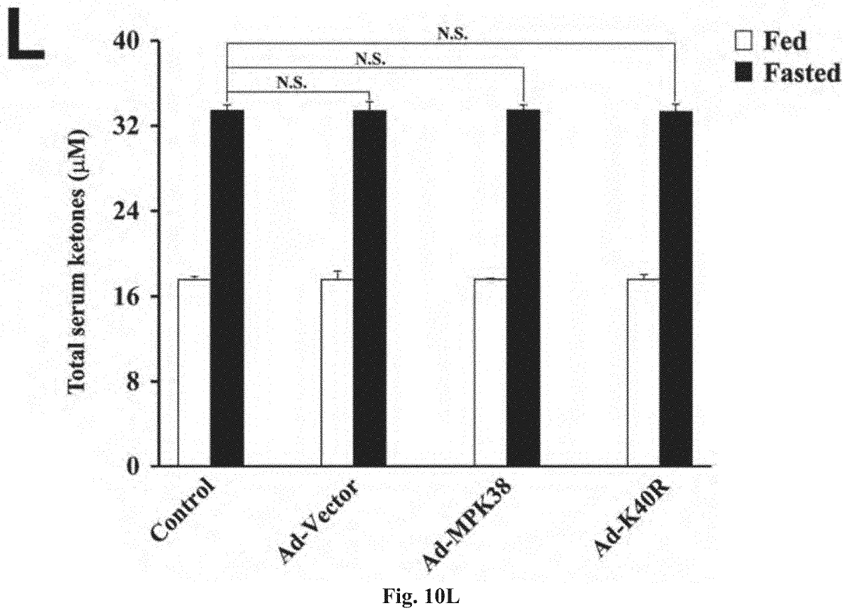

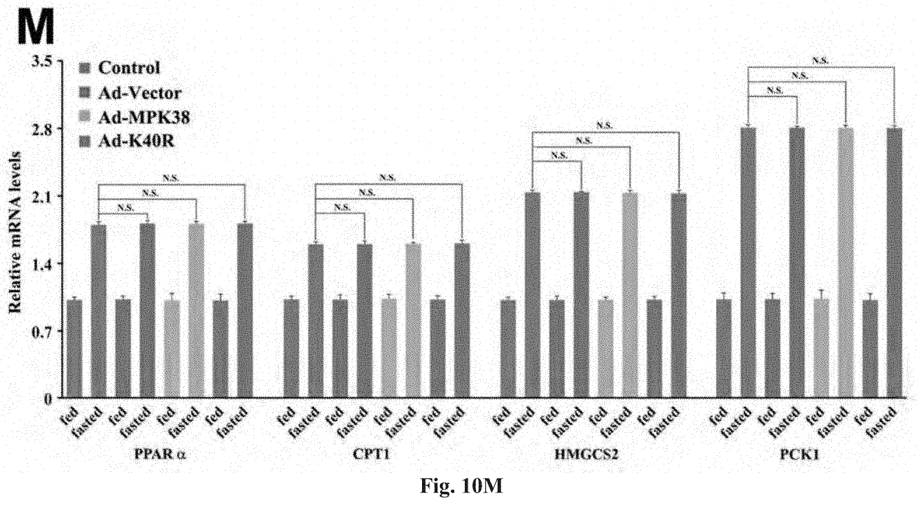

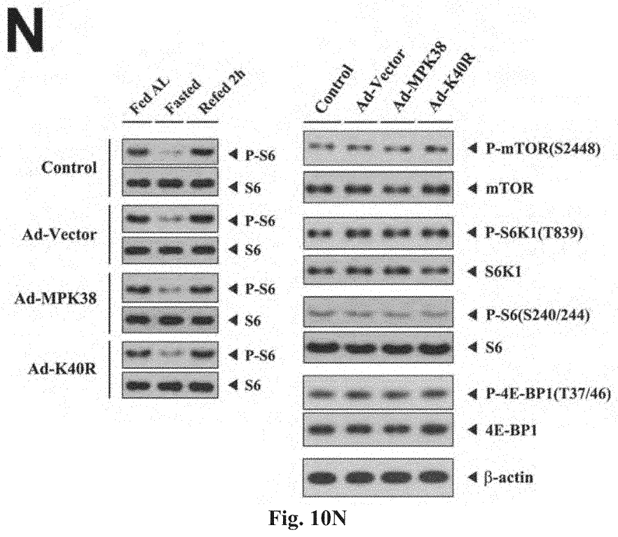

Meanwhile, the effects of ameliorating obesity and lipid metabolism disorders through induction of MPK38 expression in the high-fat diet-fed middle-aged male mice as described above did not appear in female mice of the same age (FIG. 10).

Accordingly, these results showed that the present inventors found that the protective, ameliorative and therapeutic effects of MPK38 on obesity and lipid metabolism disorders differ according to gender, and that MPK38 is specifically active for middle-aged men.

### Example 4

Analysis of effects of MPK38 on metabolic changes in middle-aged male mice by sterilization

Sexual dimorphism regarding obesity was analyzed in middle-aged MPK38-knockout MPK38^{-/-} mice. Since sterilization surgery may affect glucose and lipid metabolisms, normal mice (WT MPK38^{+/+}) and MPK38^{-/-} mice of the same age and gender were subjected to sterilization (castration or ovariectomy) or sham surgery. 6 weeks later, glucose and lipid metabolisms were analyzed.

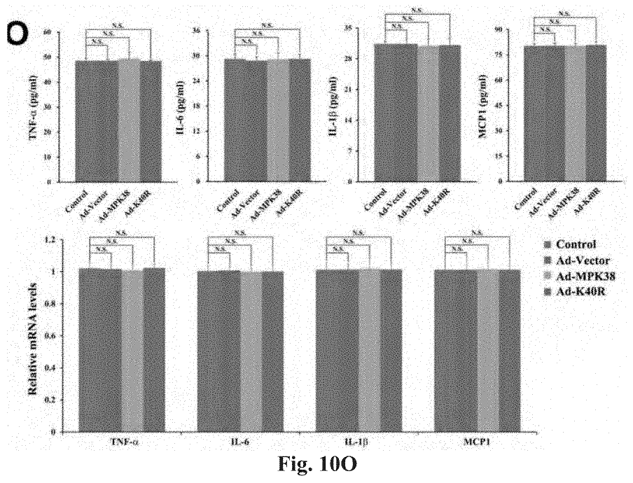

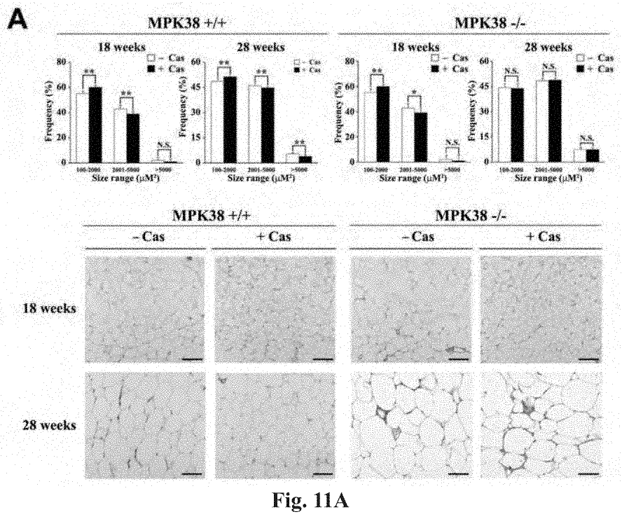

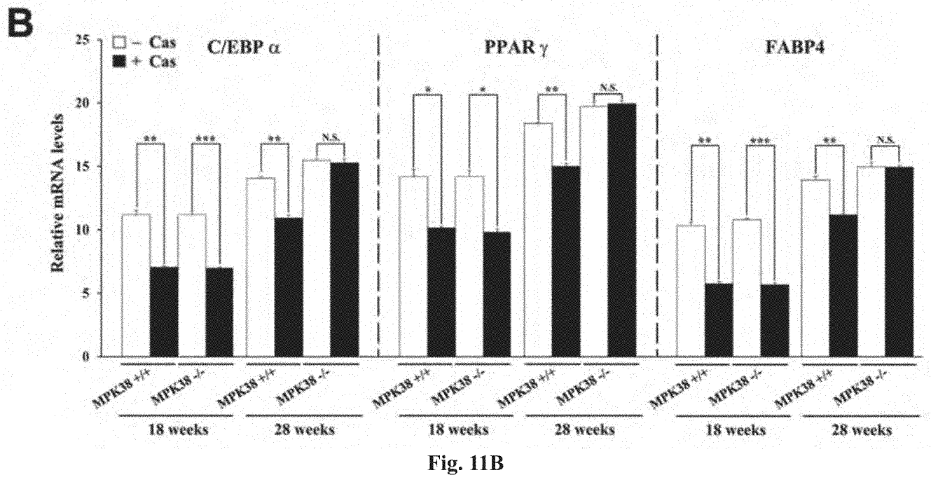

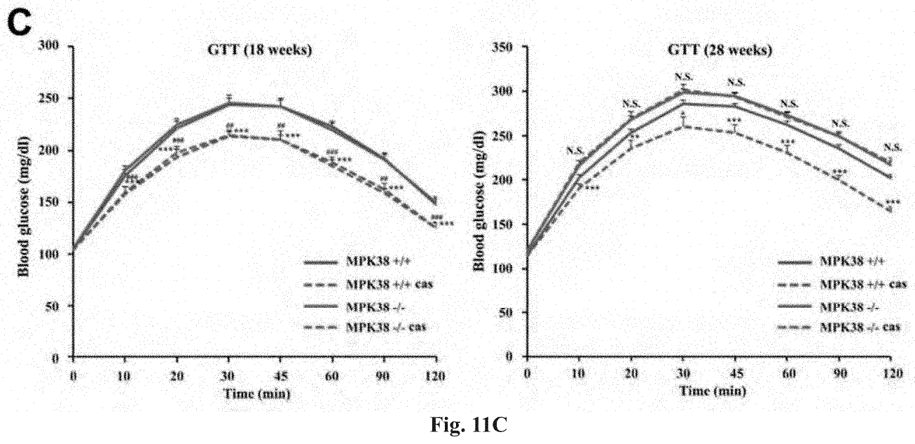

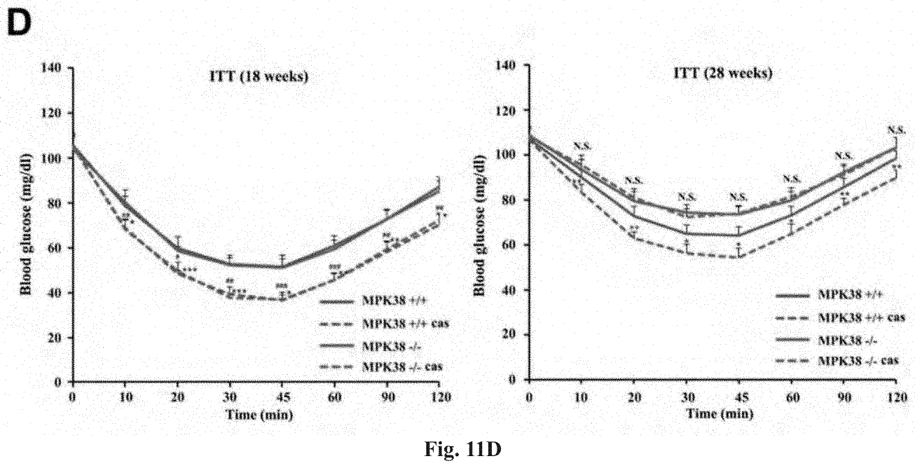

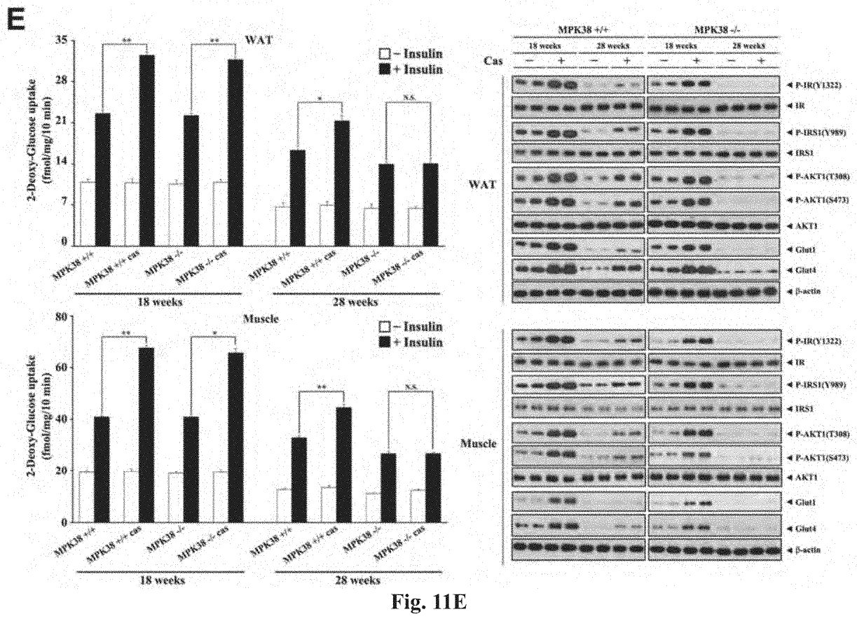

The result of histological analysis of WAT showed that castration of 7-month-old MPK38^{-/-} male mice has little effect on the adipocyte size distribution, and also has little effect on mRNA expression of major adipogenic regulators (C/EBPα, PPARγ and FABP4) (FIGS. 11A and 11B).

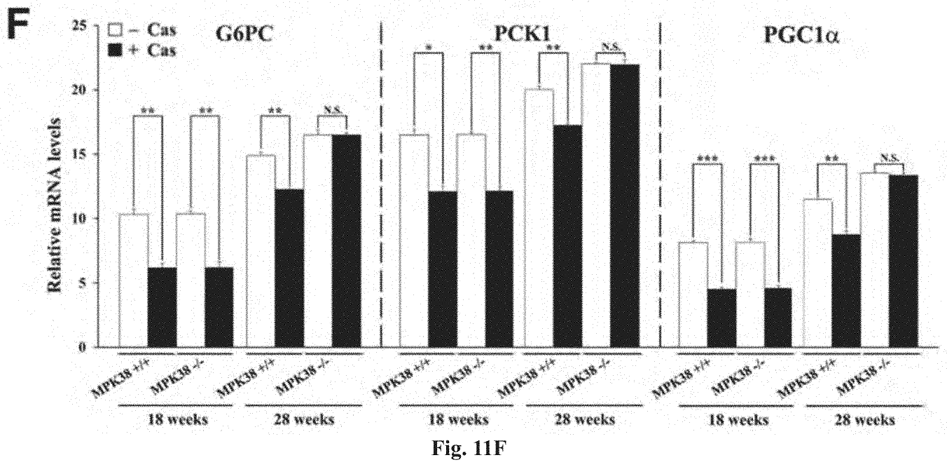

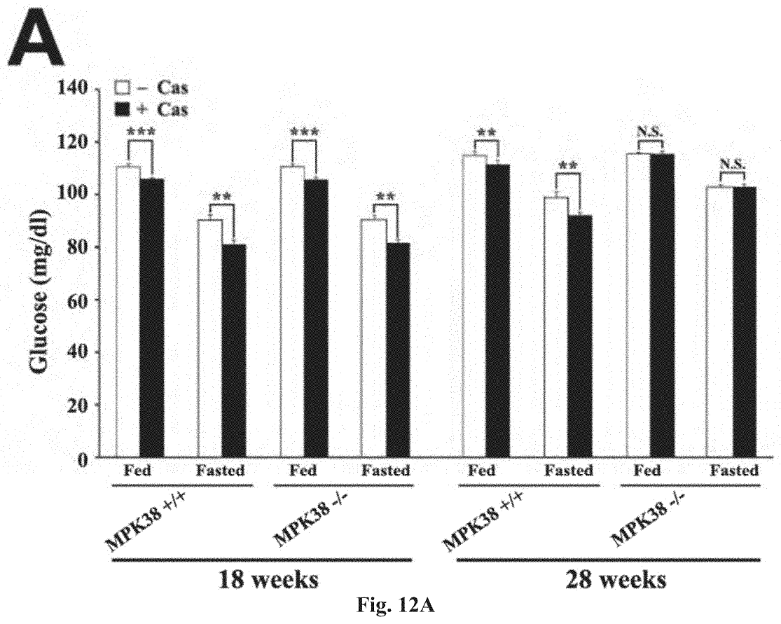

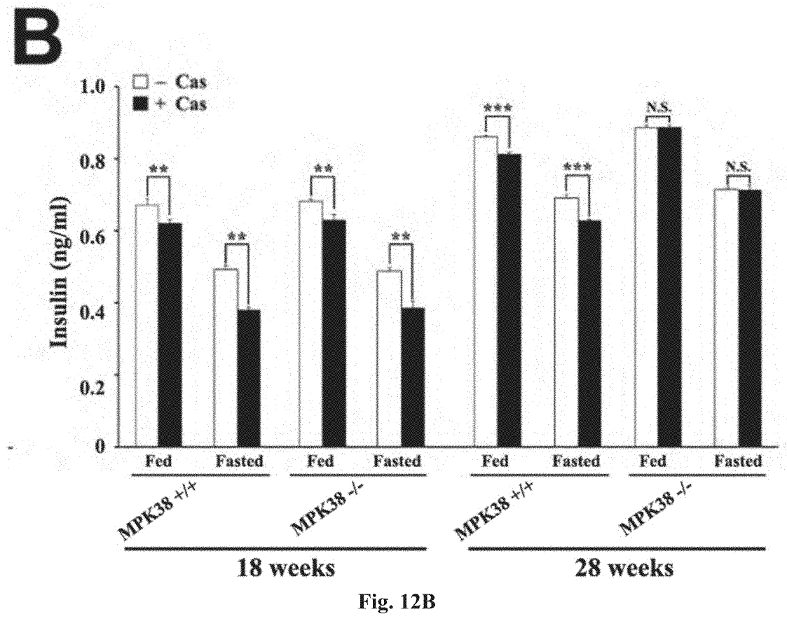

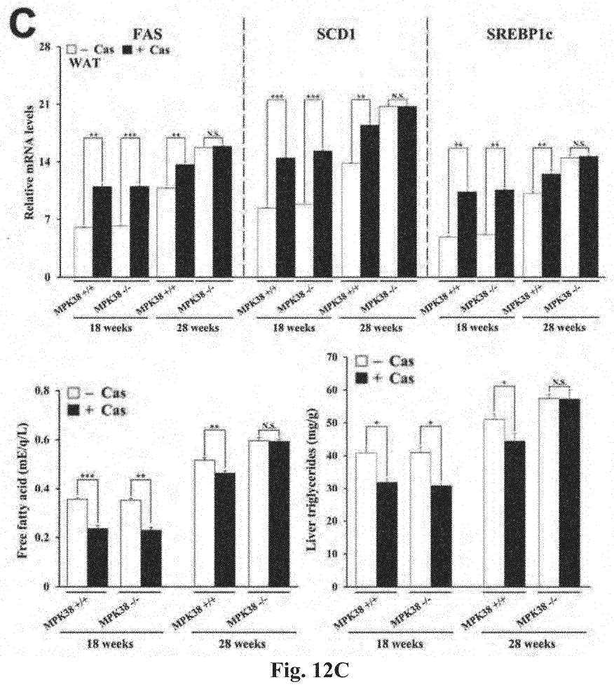

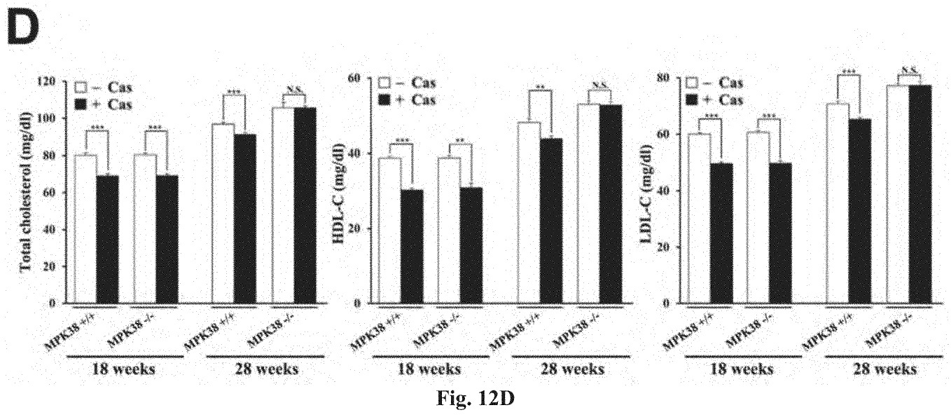

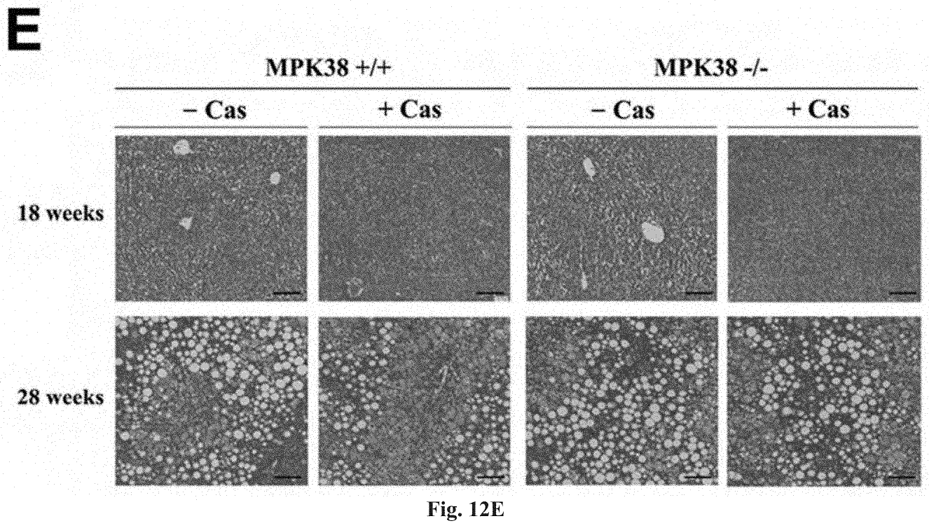

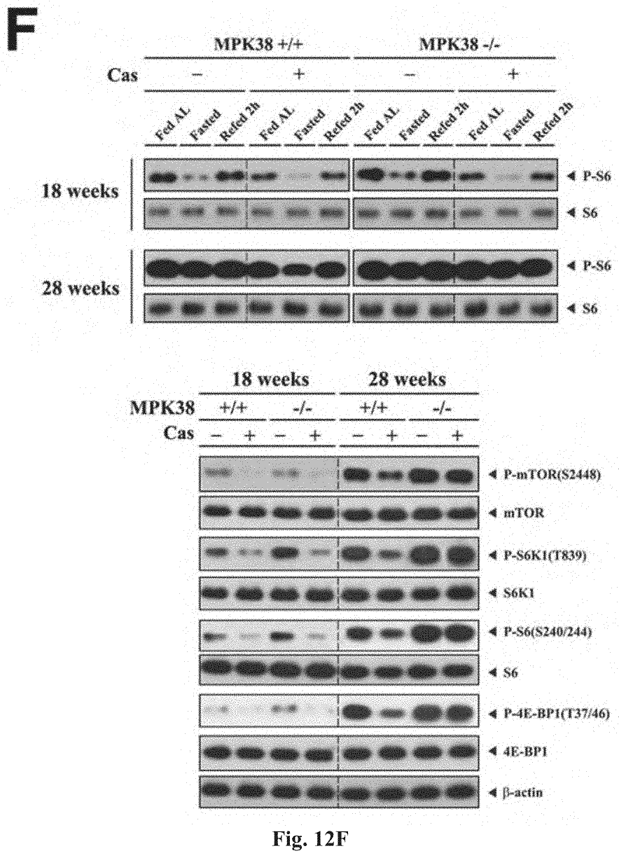

Meanwhile, normal castrated mice exhibited increased glucose tolerance and insulin sensitivity compared to sham-castrated mice (FIGS. 11C and 11D) and decreased blood insulin and glucose in the fasting state (FIGS. 12A and 12B). However, this effect was not observed in castrated 7-month-old MPK38^{-/-} male mice. In addition, the castration procedure does not cause changes in the absorption of 2-deoxy-glucose induced by insulin stimulation (FIG. 11E left), activation of the IRS-PI3K pathway (FIG. 11E right), or expression of gluconeogenic genes (G6PC, PCK1, PGC1α) in the liver (FIG. 11F).

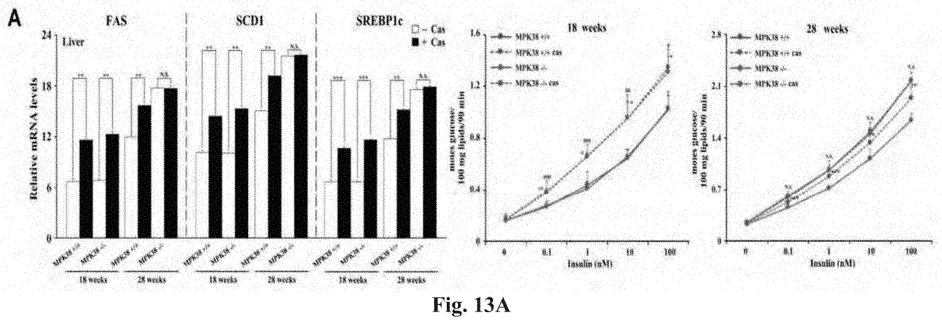

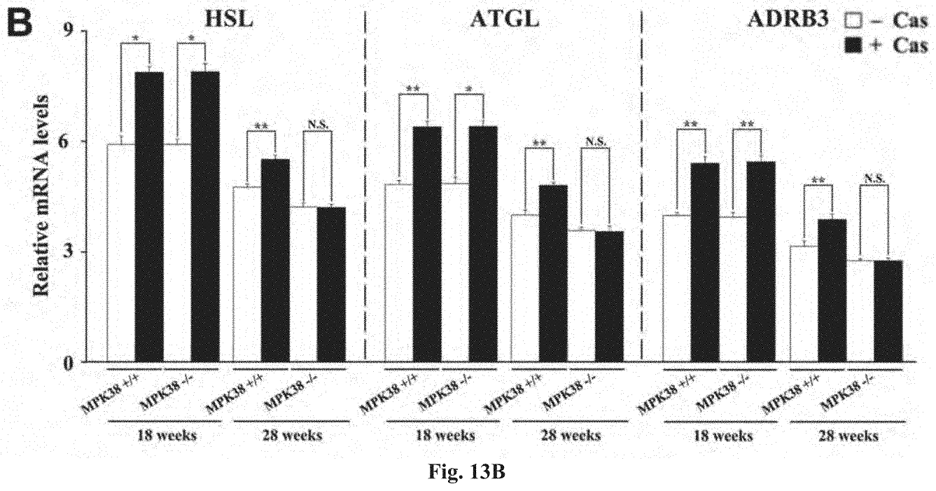

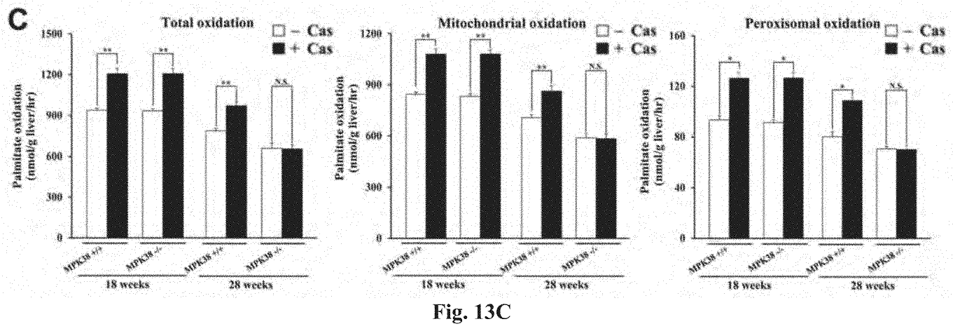

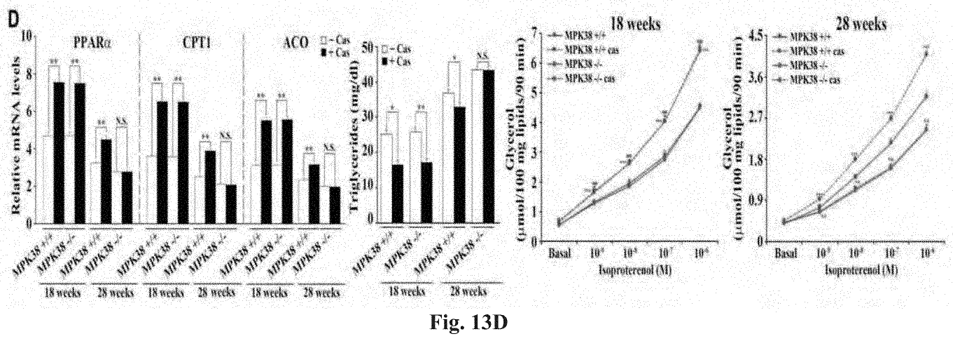

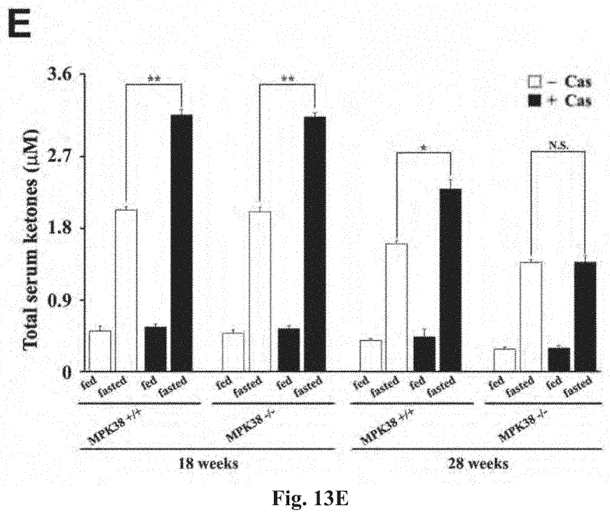

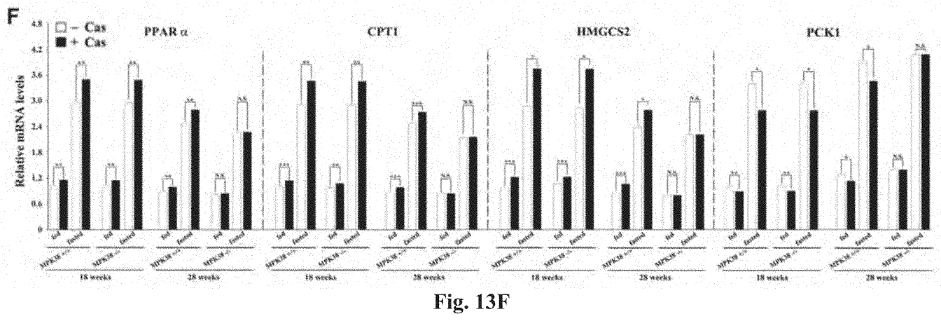

In addition, compared to the sham-castrated control mice, the castrated normal male mice exhibited faster fat production and increased mRNA expression of hepatic lipogenic genes (FAS, SCD1 and SREBP1c), but lowered concentration of FFA in the blood (FIGS. 13A and 12C). Meanwhile, this change did not appear in the 7-month-old middle-aged, castrated MPK38^{-/-} male mice, and there was no effect on liver triglyceride storage, or blood total cholesterol, HDL-C or LDL-C concentration (FIGS. 12C and 12D).

In addition, as a result of analyzing the effects of castration on the expression of lipolytic genes such as HSL (hormone-sensitive lipase), ATGL (adipose triglyceride lipase) and ADRB3 (beta-3 adrenergic receptor) (FIG. 13B), the use of fatty acids (FIG. 13C), the expression of genes involved in oxidation of fatty acids, namely, PPARα, CPT1 and ACO (FIG. 13D, left), isoproterenol-stimulated lipolysis (FIG. 13D, right), contents of liver lipids and triglycerides (FIGS. 12C and 12E), and blood triglyceride concentration (FIG. 13D, middle), castration did not have a great effect in the 7-month-old middle-aged MPK38^{-/-} male mice.

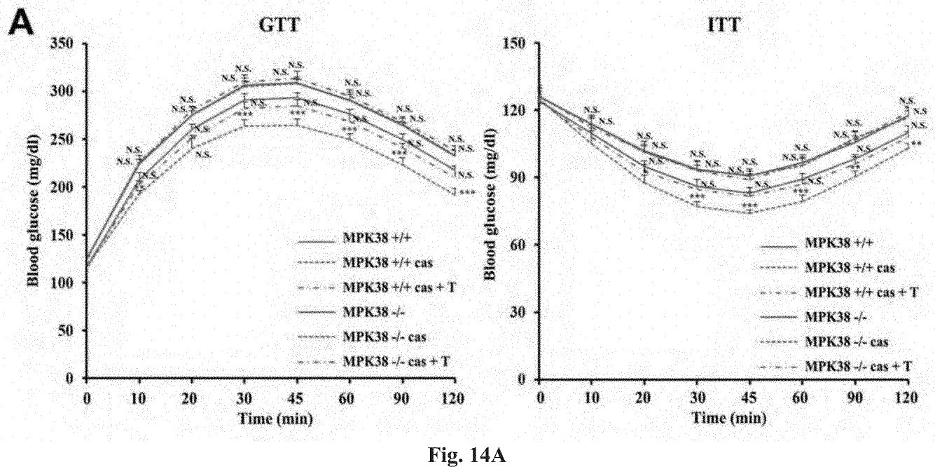

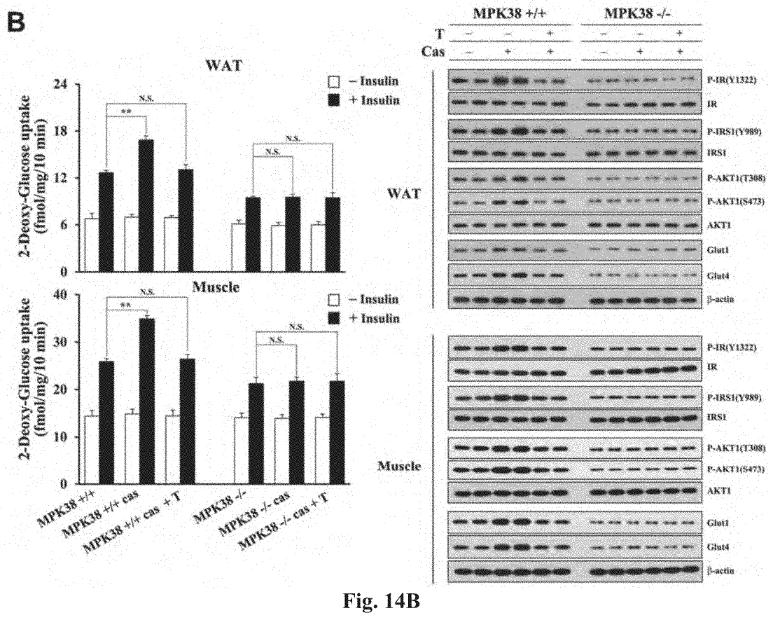

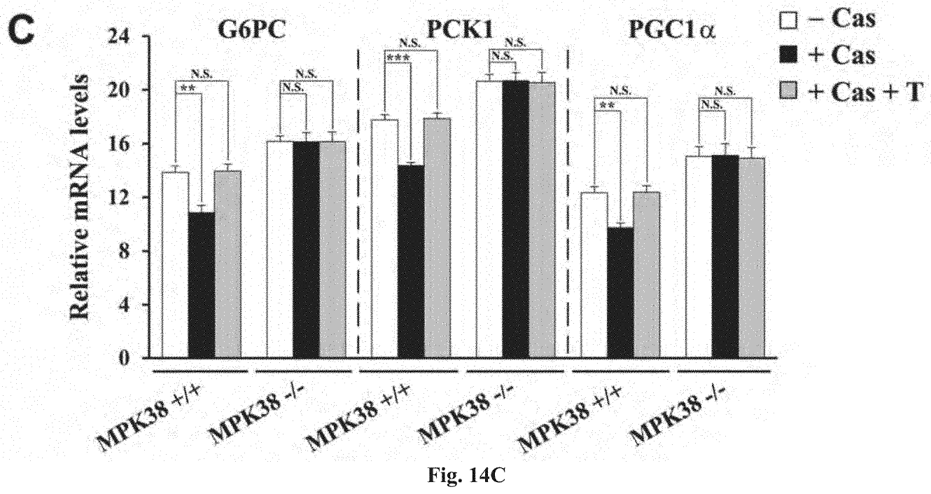

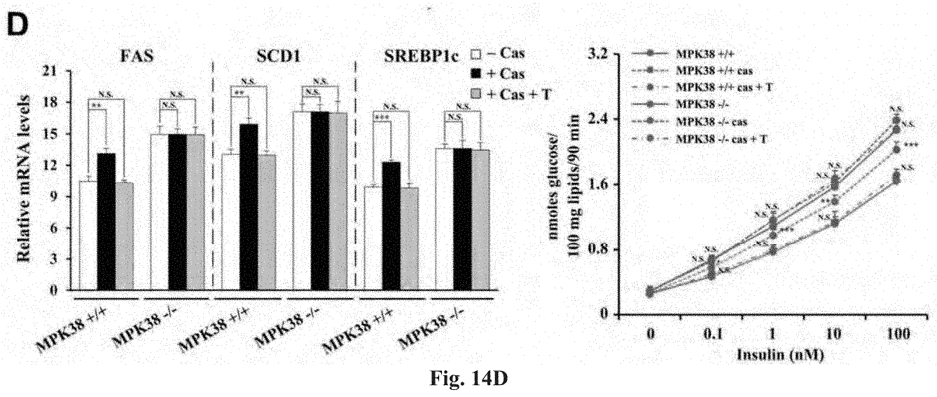

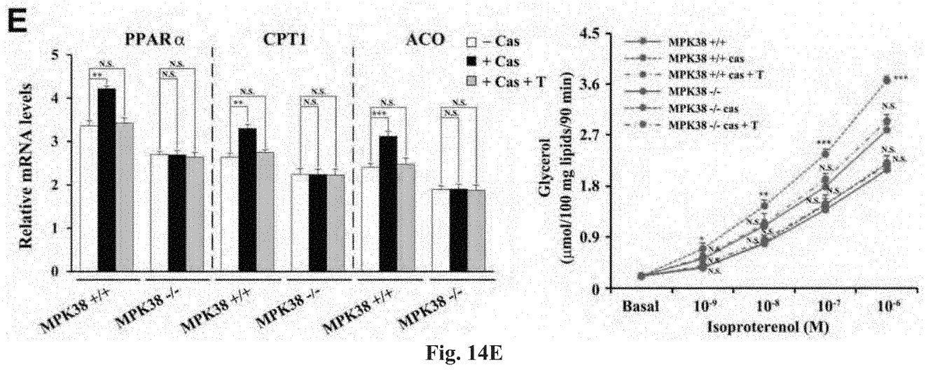

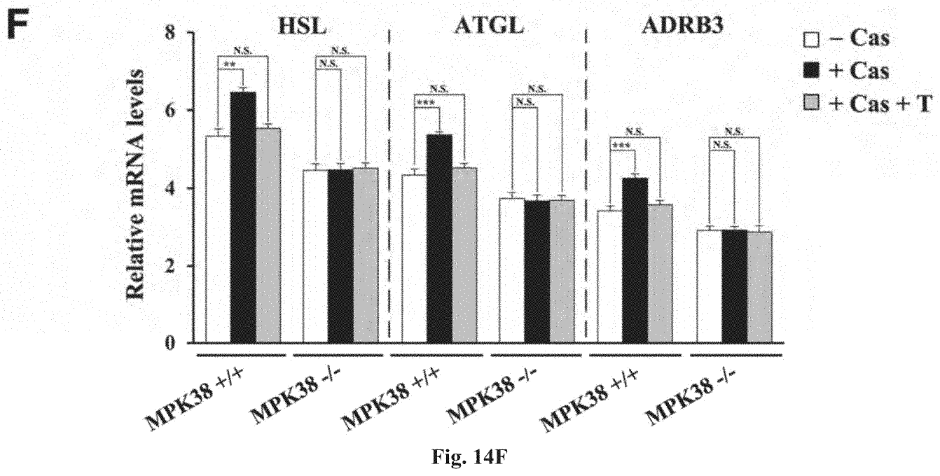

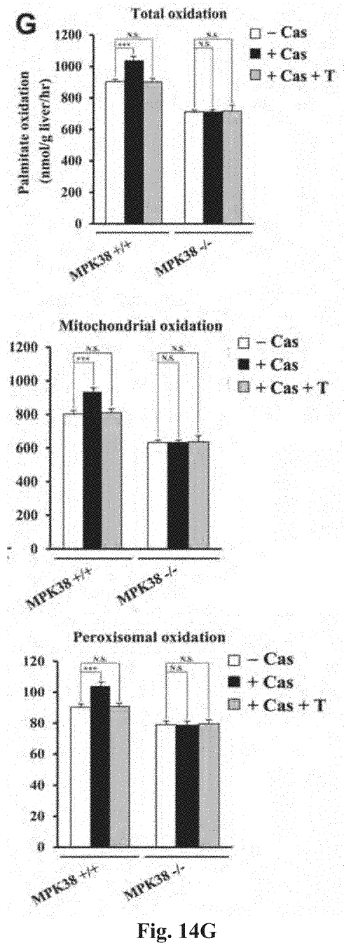

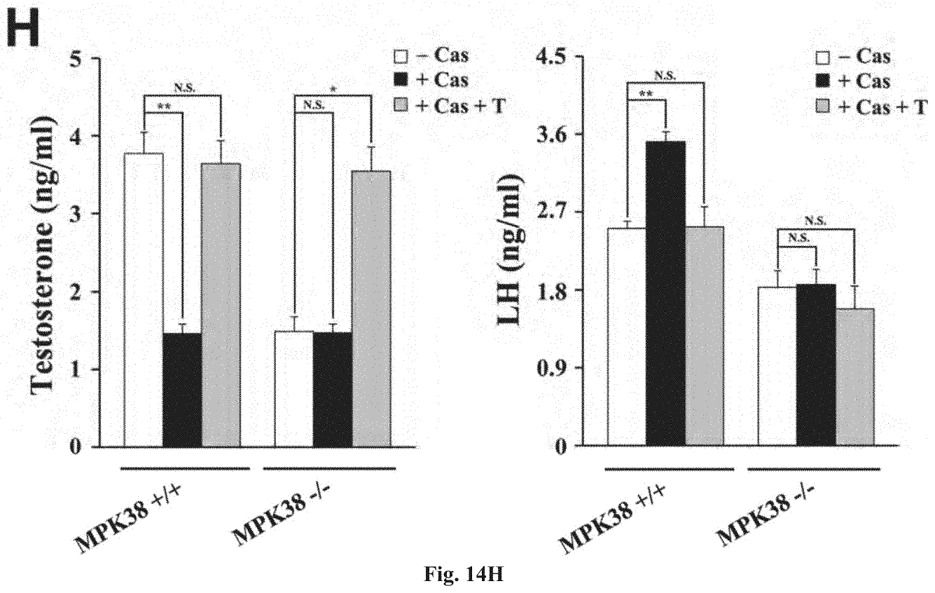

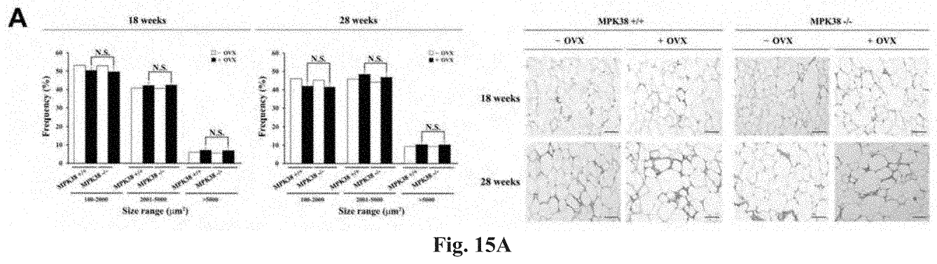

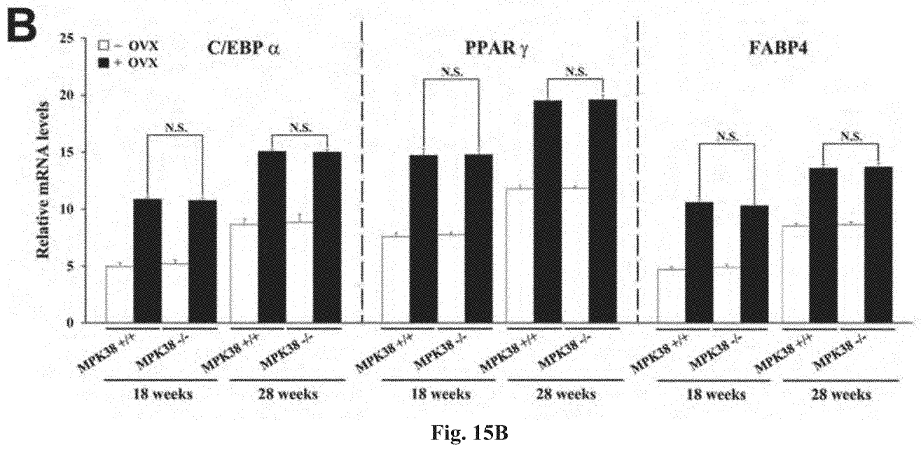

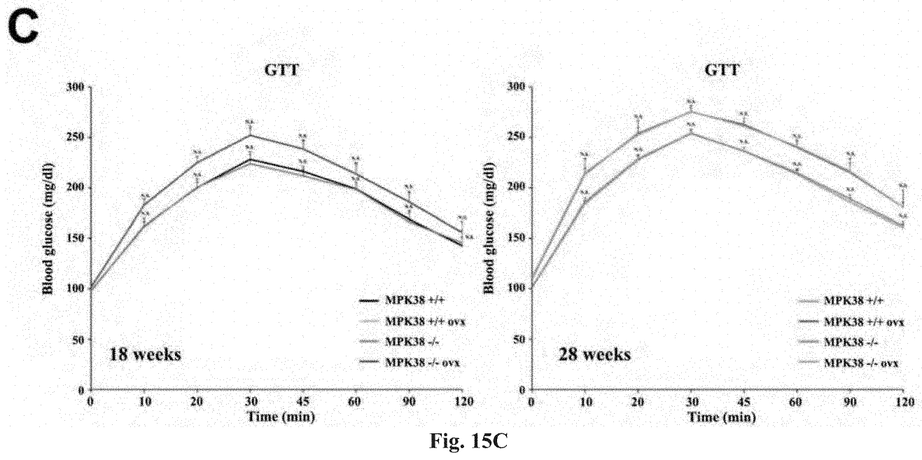

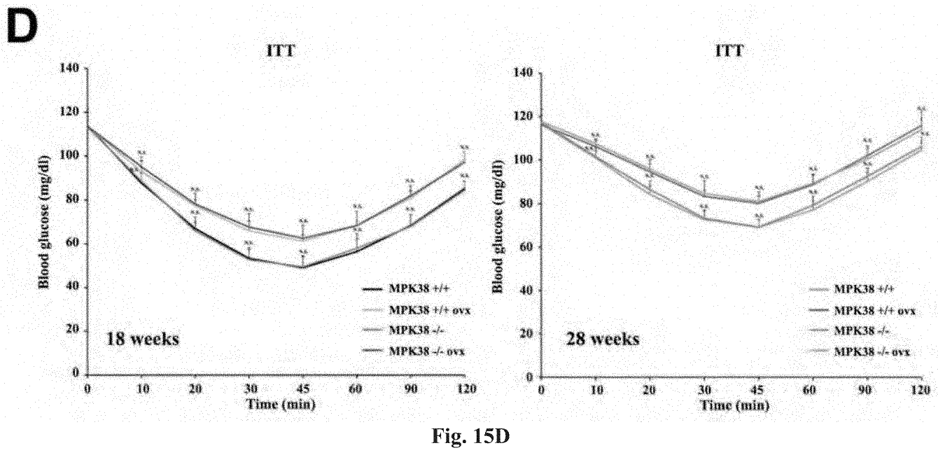

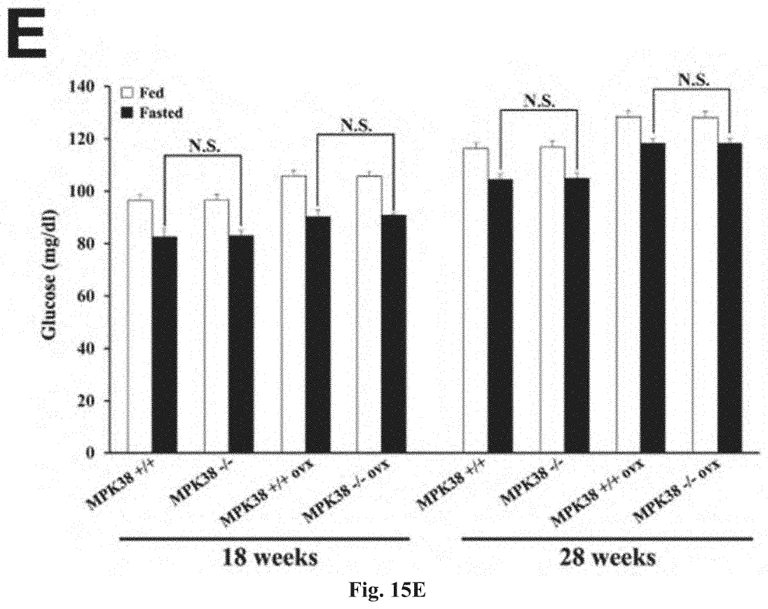

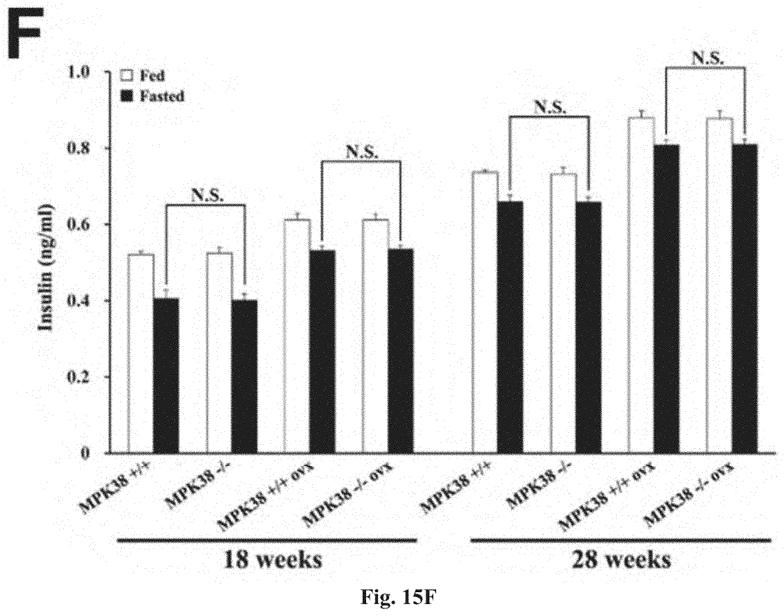

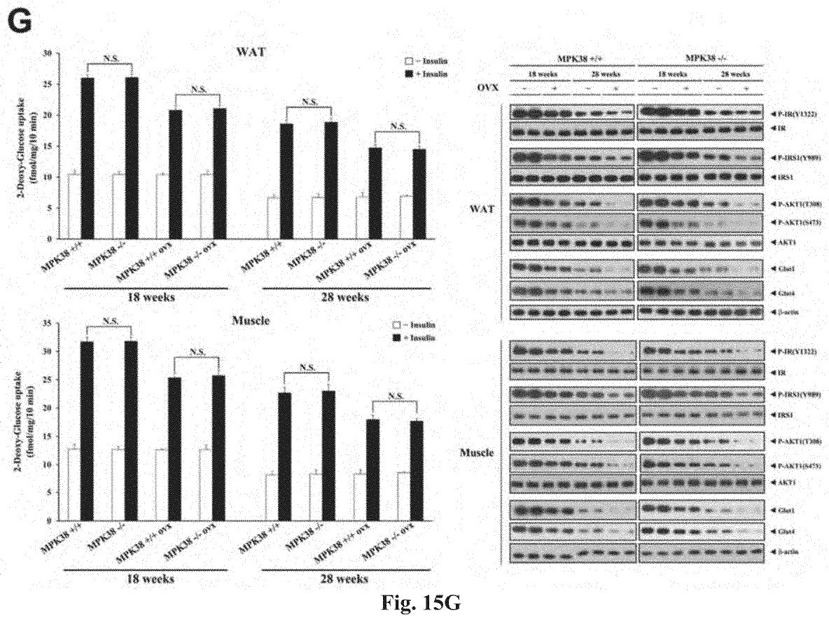

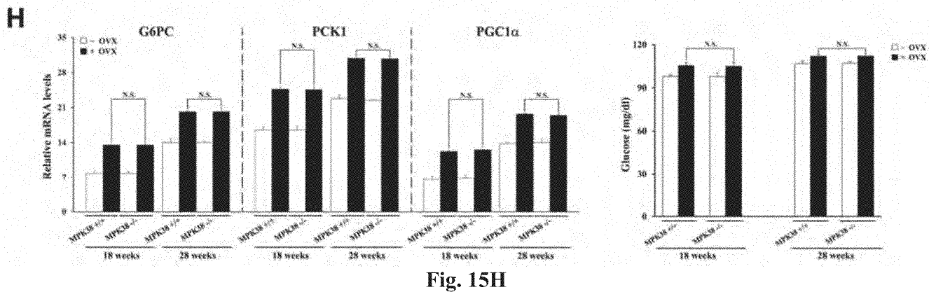

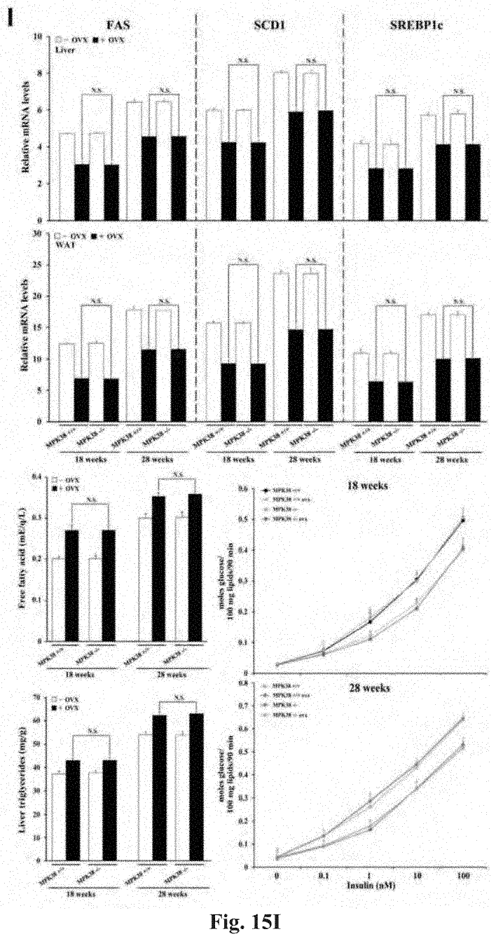

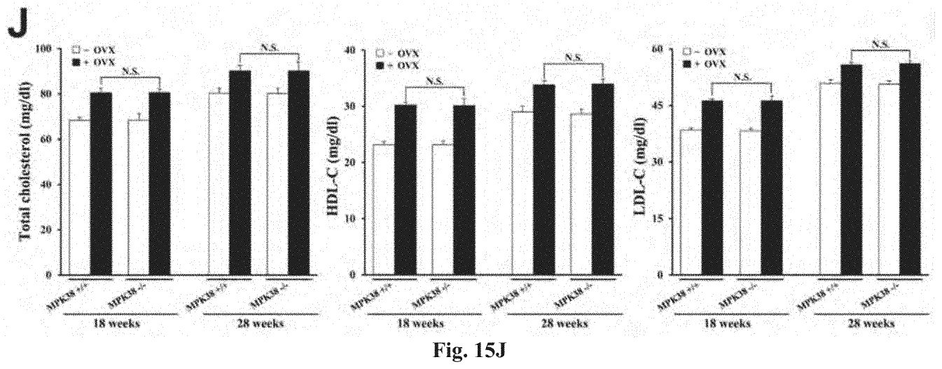

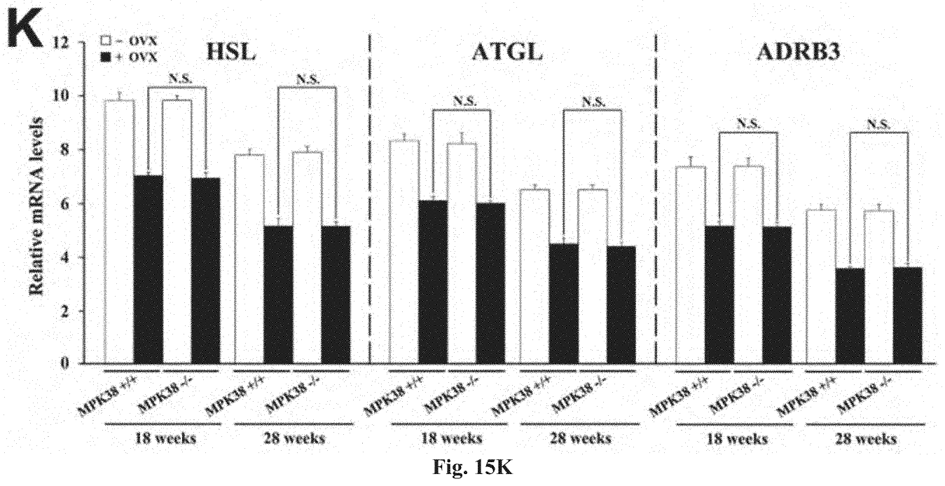

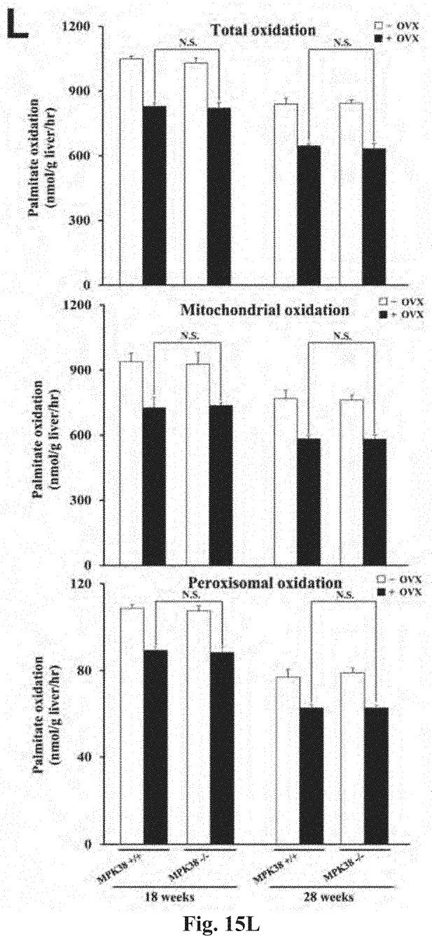

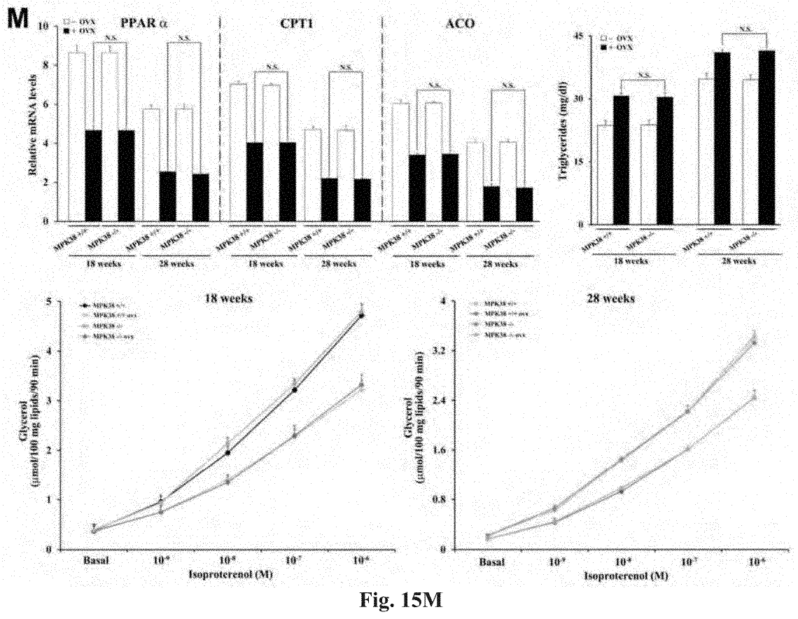

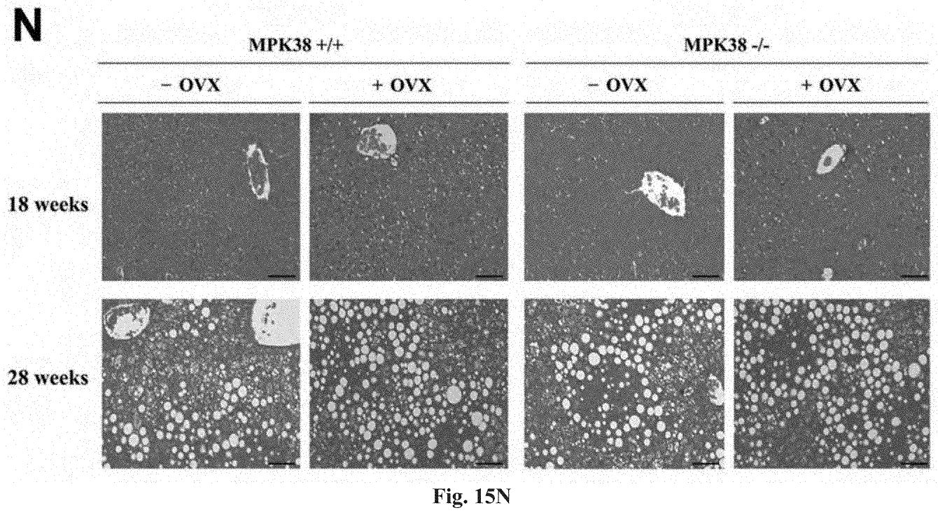

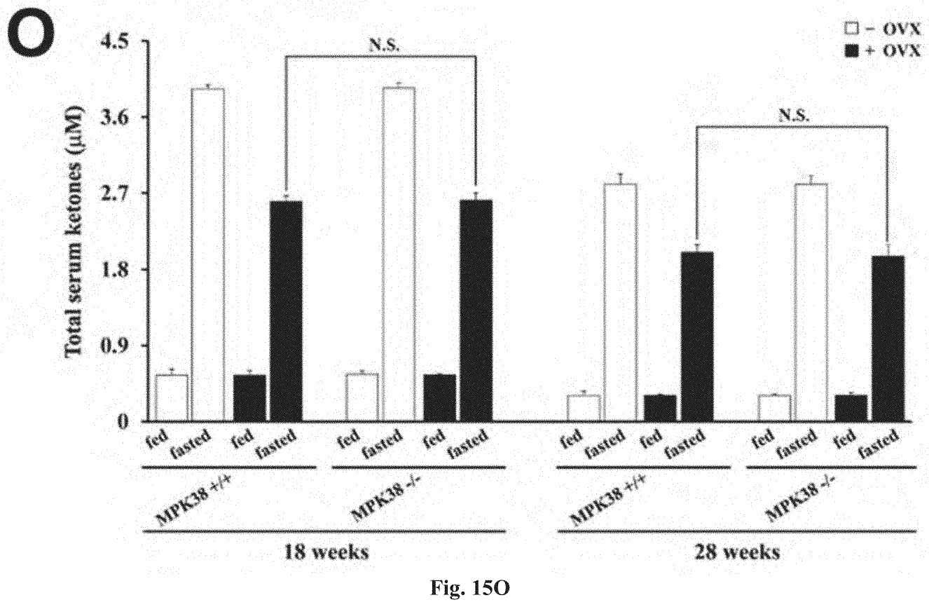

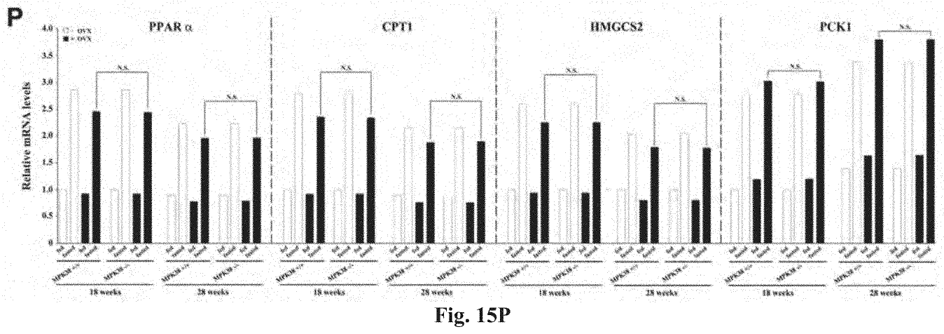

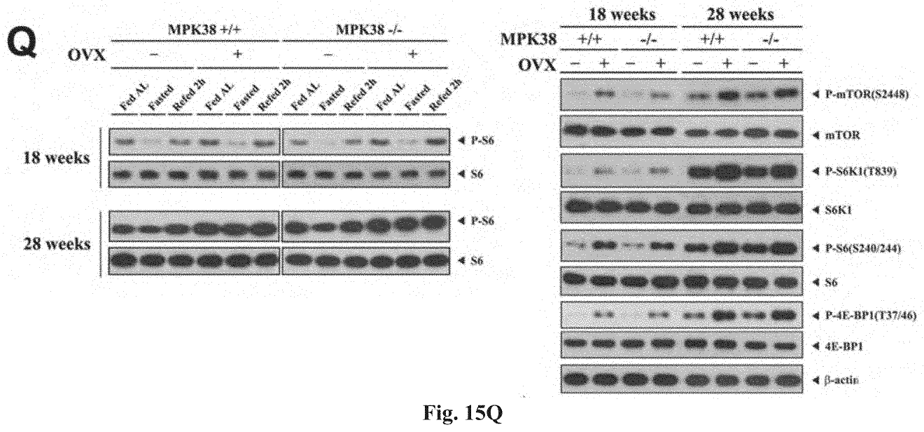

In addition, castration of normal male mice was found to greatly increase the production of ketone bodies and the expression of ketogenic genes under fasting conditions compared to the sham castration control group, but this effect was not observed in 7-month-old middle-aged castrated MPK38^{-/-} male mice (FIGS. 13E and 13F). In addition, castrated MPK38^{-/-} male mice did not exhibit a change in phosphorylation at the Ser240/244 residue of S6 in response to fasting or mTORC1 signaling-pathway activation (FIG. 12F). This was further ascertained by the results of testosterone replacement (T replacement), showing effects opposite to all of these results (FIG. 14). Meanwhile, the phenotype of castrated 7-month-old MPK38^{-/-} male mice was not observed in ovariectomized MPK38^{-/-} female mice of the same age (FIG. 15).

These results showed that the present inventors found that the effects of MPK38 on sugar and lipid metabolisms are specific to males and specific to age (middle-aged and elderly).

### Example 5

Analysis of Effects of MPK38 on Male Testosterone Production

Since AMPK regulates the synthesis of steroid hormones, the contents of testosterone and estrogen in the serum in an AMPK-like protein, MPK38 were assayed through LC-MS/MS analysis. At this time, hormone assay was performed in sham-castrated normal male mice and MPK38^{-/-} male mice, and castrated normal male mice and MPK38^{-/-} male mice, respectively.

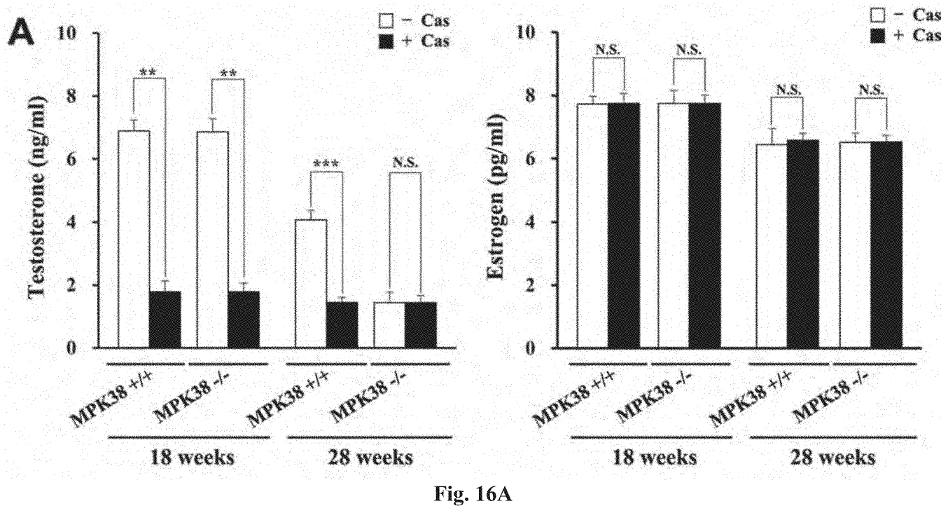

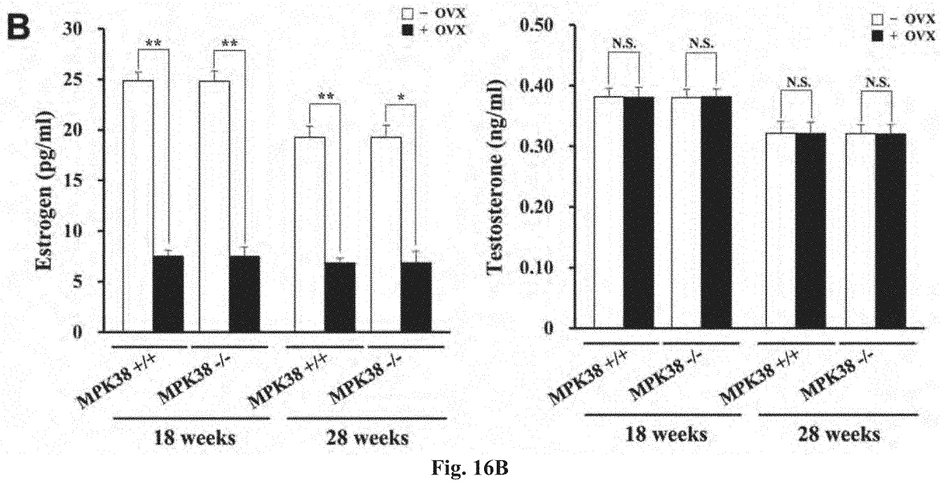

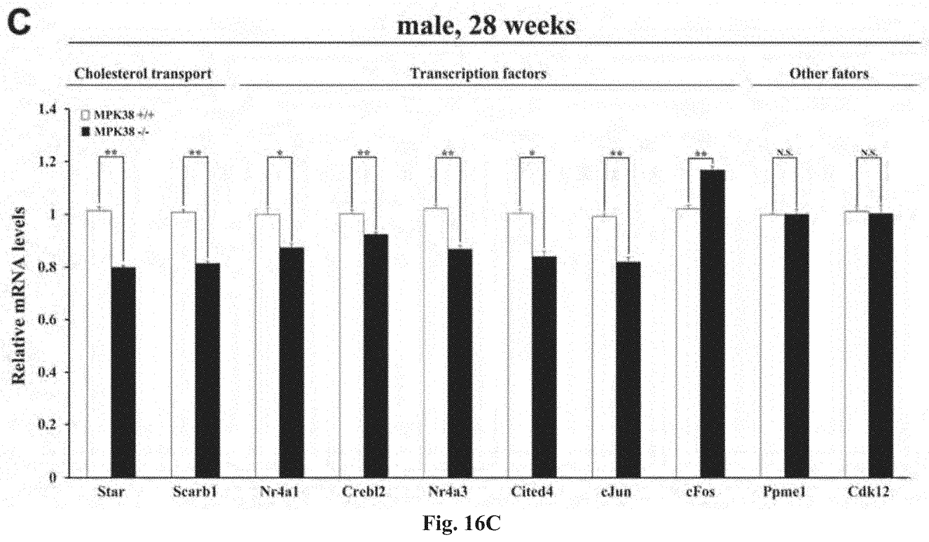

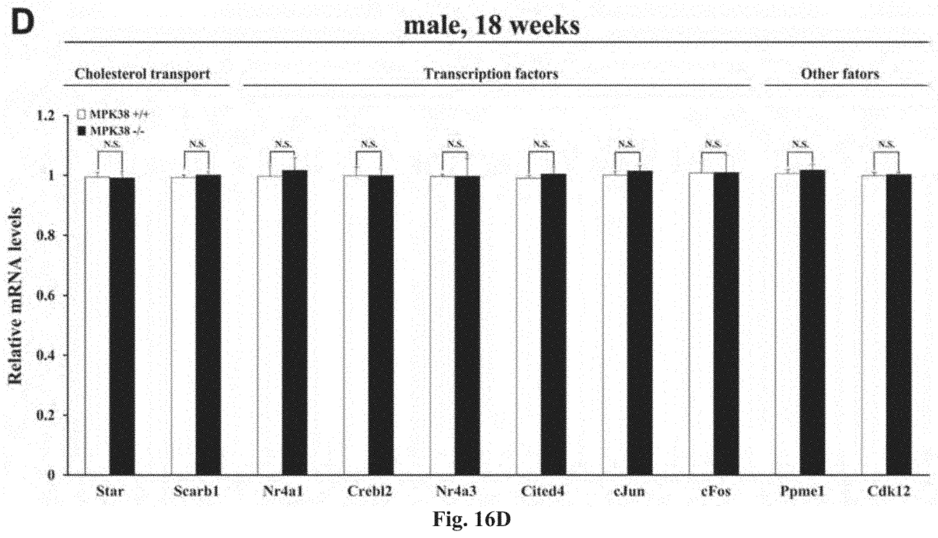

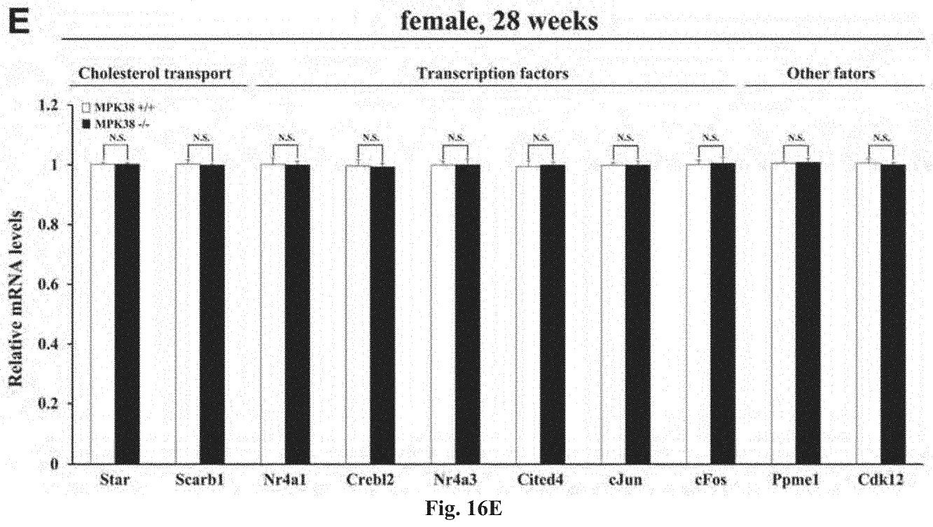

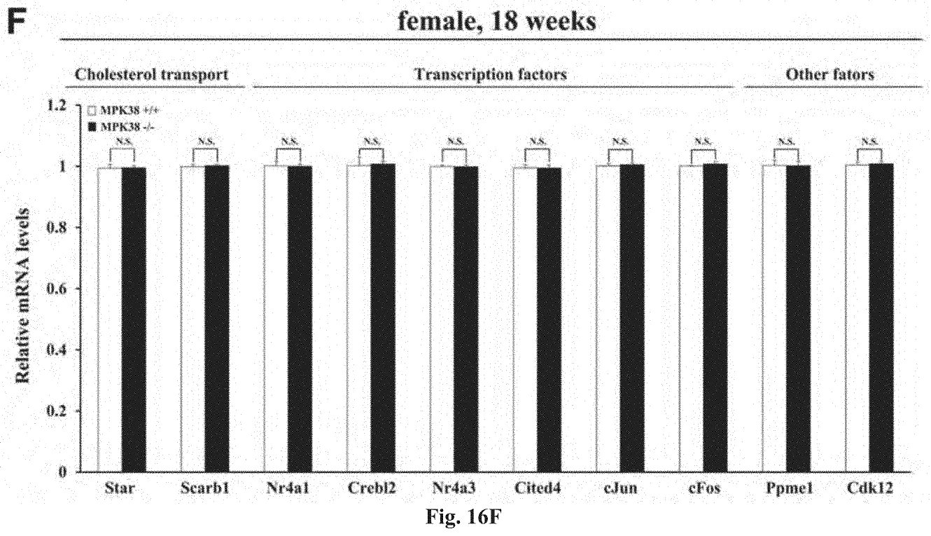

The result showed that the content of testosterone in the serum of 7-month-old uncastrated MPK38^{-/-} male mice was similar to that of normal castrated male mice of the same age (FIG. 16A, left). However, there was no significant difference in estrogen content (FIG. 16A, right).

Meanwhile, this effect could not be observed in ovariectomized MPK38^{-/-} female mice of the same age (FIG. 16B left). As expected, ovariectomy was found to have no effect on the change of testosterone content in female mice (FIG. 16B, right).

Based on these results, the present inventors found that MPK38 specifically affects the production of testosterone, a male hormone, in middle-aged or elderly adult men.

Next, the present inventors conducted an experiment to determine whether or not the change in the expression of the steroid-producing genes is related to the regulation of testosterone production mediated by MPK38. For this purpose, RNA was extracted from the testes of each of normal-diet-fed 7-month-old normal male mice and MPK38^{-/-} male mice, gene expression levels were analyzed through quantitative PCR, and the analyzed genes were Star (steroidogenic acute regulatory protein) and Scarb1 (scavenger receptor b1) as cholesterol transporters, Nr4a1, Nr4a3, Creb12, Cited4 and cJun as transcription activators, and Ppme1 (protein phosphatase methylesterase 1) and Cdk12 (cyclin-dependent kinase 12).

The result showed that the expression of Star and Scarb1, and Nr4a1, Nr4a3, Creb12, Cited4 and cJun5 as transcription activators was lower in MPK38^{-/-} male mice than in normal male mice, whereas the expression of c-Fos, a steroid-producing inhibitor, was higher in MPK38^{-/-} male mice than in normal male mice, and the expression of Ppme1 and Cdk12 used as controls was not affected by genotype (FIG. 16C). On the other hand, this difference in gene expression was not observed in 4.5-month-old MPK38^{-/-} male mice or normal male mice (FIG. 16D).

In addition, similar results to those as described above were not observed regardless of age in the ovaries of normal and MPK38^{-/-} female mice. This indicates that MPK38 had no effect on estrogen production in women (FIGS. 16E and 16F).

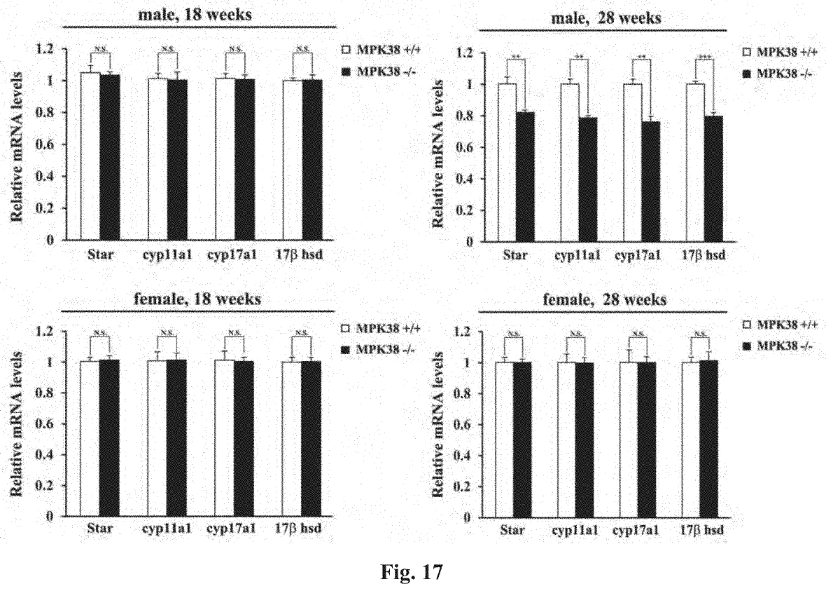

Meanwhile, results similar to those described above were observed in analysis of steroid genes including Cyp11a1 (cytochrome P450 family 11 subfamily A member 1), Cyp17a1 (cytochrome P450 family 17 subfamily A member 1), and 17β-hsd (17β-hydroxysteroid dehydrogenase), which are factors involved in steroid synthesis (FIG. 17).

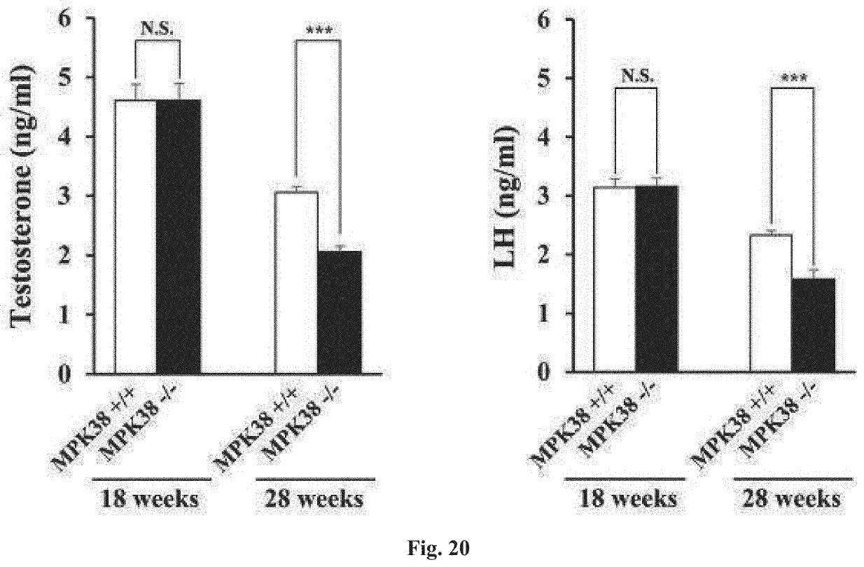

In addition, the content of testosterone and luteinizing hormone (LH) was analyzed in each of MPK38-knockout 4.5-month-old male mice, MPK38-knockout 7-month-old male mice, MPK38-expressing 4.5-month-old male mice, and MPK38-expressing 7-month-old male mice. As a result, as can be seen from FIG. 20, the 7-month-old MPK38^{-/-} male mice exhibited the lowest testosterone production and decreased luteinizing hormone production. This means that MPK38 deficiency affects the hypothalamus-pituitary-gonadotropin axis and leads to obesity and diseases such as central hypogonadism, a metabolic disease related to obesity.

These results showed that the present inventors found that MPK38 specifically contributes to the production of testosterone in mature adult males (middle-aged males), while MPK38 does not contribute to estrogen production in females of the same age.

### Example 6

Analysis of Stability of MPK38 by Testosterone and Estrogen

In order to determine whether or not sex hormones such as testosterone and estrogen affect the kinase activity and expression of MPK38, immunoblot analysis was performed on adipocytes derived from 4.5-month-old normal male and female mice treated with DHT and E2.

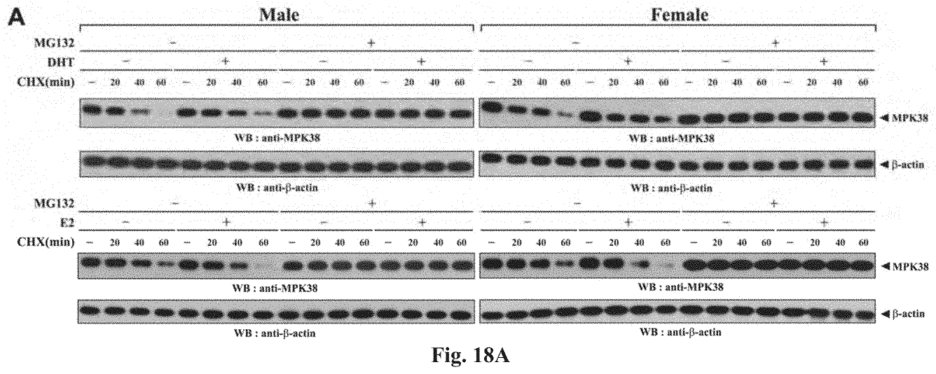

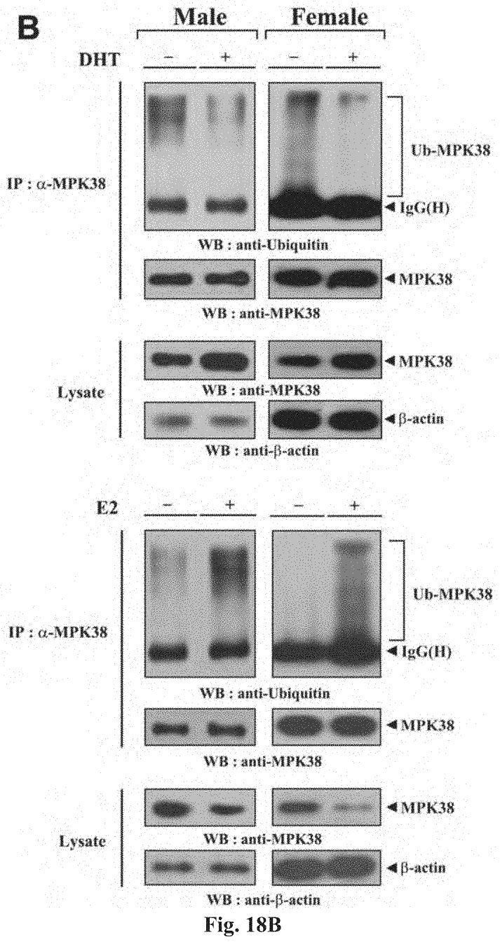

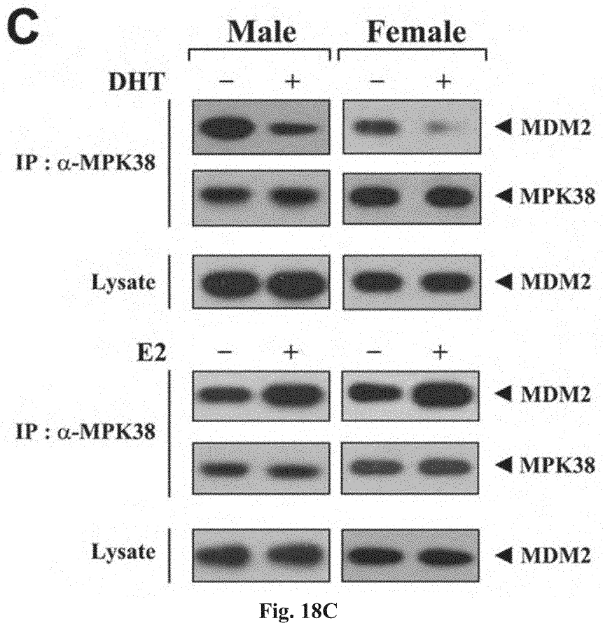

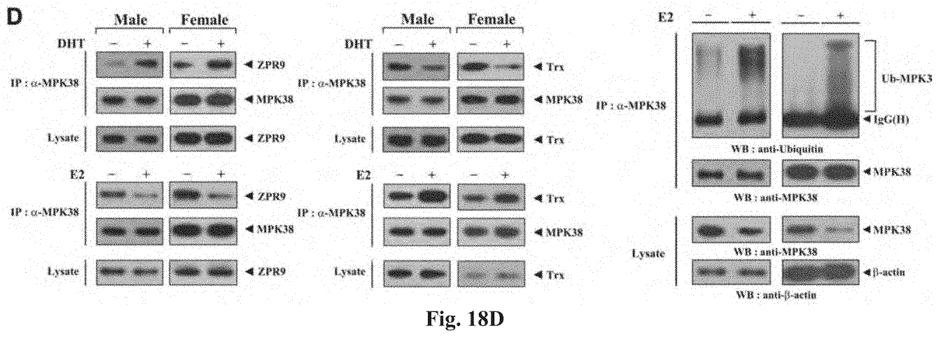

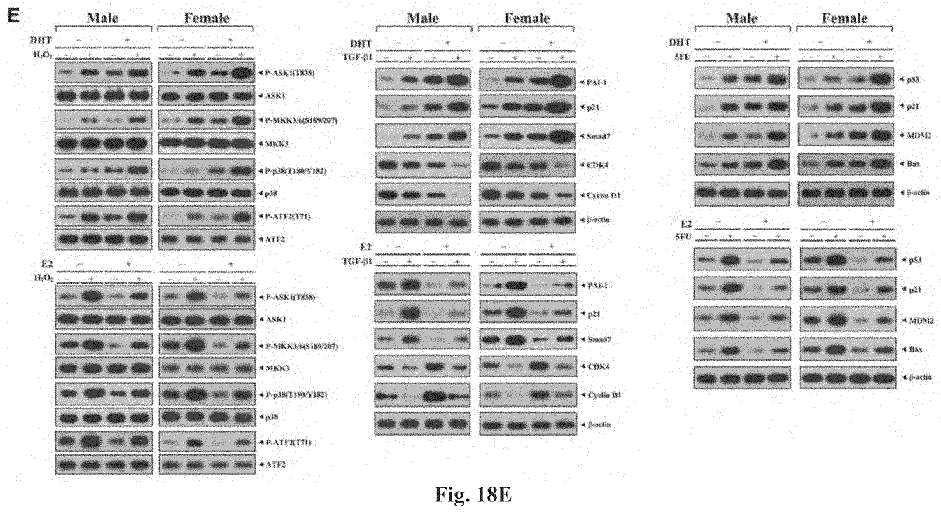

The result showed that the stability of MPK38 was increased in the group treated with DHT, while the stability of MPK38 was decreased in the group treated with E2 (FIG. 18A). However, when the cells were exposed to MG132 as a proteasome inhibitor, the stability of MPK38 was found to increase in the treated group compared to the untreated group (FIG. 8A). These results mean that the proteasome signaling pathway is involved in the degradation of MPK38. Consistent therewith, the endogenous ubiquitination of MPK38 decreased in the DHT-treated group and increased in the E2-treated group, and this occurs regardless of gender (FIG. 18B).

Furthermore, since MPK38 physically interacts with Mdm2, whether or not DHT and E2 are involved in the decomposition of MPK38 mediated by Mdm2 was determined.

The result showed that the extent of formation of an endogenous MPK38-Mdm2 complex was decreased in the DHT-treated group compared to the control group not treated with DHT or E2, whereas the formation of the complex was increased in the E2-treated group (FIG. 18C).

In order to further determine whether or not testosterone increases the stability of MPK38, the effect of DHT on the formation of a complex of MPK38 with either ZPR9 or Trx was analyzed. The reason for this is that ZPR9 or Trx acts as a stabilizer or destabilizer of MPK38.

The result of analysis showed that the formation of an endogenous MPK38-ZPR9 complex was increased in the DHT-treated group, whereas the formation of the MPK38-Trx complex was decreased in the DHT-treated group, and this was the same for both females and males (FIG. 18D). However, treatment with E2 yields results opposite those of treatment with DHT.

In addition, the present inventors determined whether or not DHT or E2 had an effect on ASK1/TGF-β/p53 signaling induced MPK38. Compared to the control group not treated with DHT or E2, the group treated with DHT exhibited increased ASK1/TGF-β/p53 signaling, whereas the group treated with E2 exhibited decreased ASK1/TGF-β/p53 signaling (FIG. 18E).

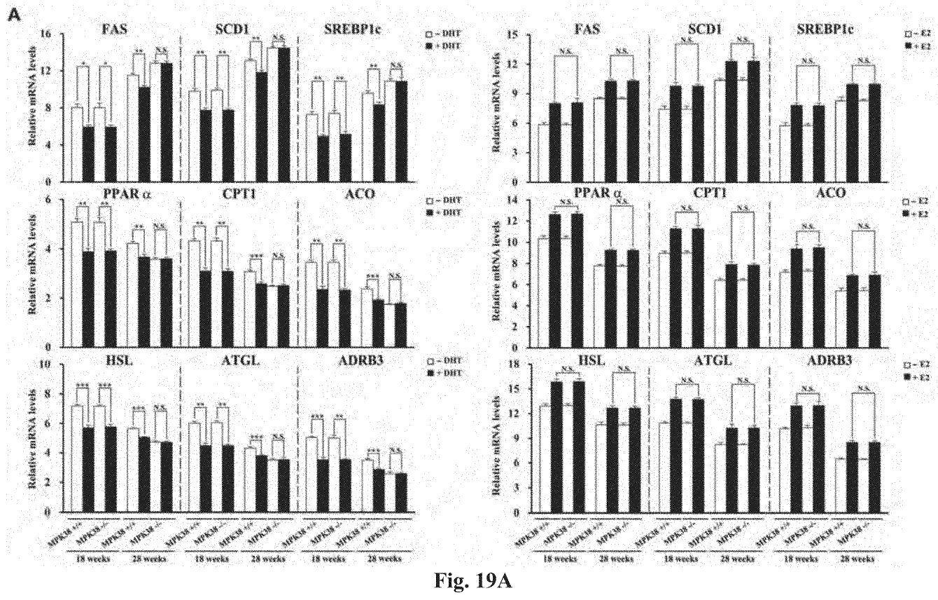

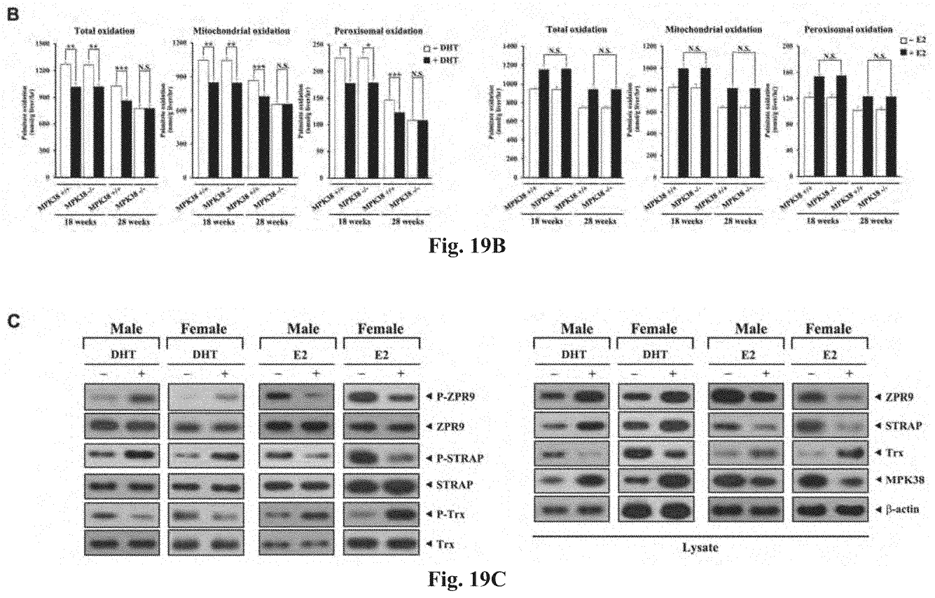

The effects of DHT and E2 on lipid metabolism and MPK38 kinase activity were also determined to be similar in liver cells, which means that the deficiency of MPK38 affects the WAT and liver equally (FIG. 19).

These results indicate that testosterone upregulates the MPK38-dependent signaling pathways of ASK1, TGF-β, and p53, whereas estrogen downregulates the signaling pathway, and that the stability of MPK38 is regulated differently in males and females.

Overall, based on these results, the present inventors found that, when the expression or activity of MPK38 is promoted or facilitated, obesity or metabolic disorders specific to middle-aged men can be effectively prevented, ameliorated or treated.

As is apparent from the foregoing, in the present invention, obesity and metabolic disorders were found to occur in MPK38-knockout middle-aged male mice. As a result of inducing the expression of MPK38 in MPK38-knockout middle-aged male mice, MPK38 was found to have effects of decreasing the size of adipocytes, suppressing the expression of adipogenic genes, reducing glucose and insulin in the blood, enhancing insulin sensitivity, reducing triglycerides and total cholesterol in the blood, promoting ketone formation, and promoting the production of testosterone, a male hormone, in middle-aged male mice. In particular, it was found that the effects of the MPK38 of the present invention are specific to middle-aged men and that MPK38 is useful as a therapeutic agent and health functional food for treating obesity or metabolic diseases specific to middle-aged men.

Although the preferred embodiments of the present invention have been disclosed, those skilled in the art will appreciate that various modifications, additions and substitutions are possible, without departing from the scope and spirit of the invention as disclosed in the accompanying claims. Therefore, the disclosed embodiments should be considered from an illustrative point of view rather than a limiting point of view. The scope of the present invention is defined by the claims rather than the aforementioned description, and all differences falling within the scope of equivalents thereto should be construed as falling within the scope of the present invention.

What is claimed is:

1. A pharmaceutical composition for preventing or treating obesity or a metabolic disease specific to middle-aged men, comprising a murine protein serine-threonine kinase 38 (MPK38) protein or a gene encoding the same as an active ingredient.

2. The pharmaceutical composition according to claim 1, wherein the protein has an amino acid sequence of SEQ ID NO: 1.

3. The pharmaceutical composition according to claim 1, wherein the gene has a nucleotide sequence of SEQ ID NO: 2.

4. The pharmaceutical composition according to claim 3, wherein the MPK38 gene having the nucleotide sequence of SEQ ID NO: 2 is inserted into an expression vector.

5. The pharmaceutical composition according to claim 1, wherein the MPK38 gene is specific to middle-aged men and reduces a size of adipocytes, inhibits expression of C/EBPα (CCAAT-enhancer-binding protein α) and PPARγ (peroxisome proliferator-activated receptor gamma and FABP4) as adipogenic genes, improves insulin sensitivity, reduces blood glucose and insulin, reduces blood triglyceride, total cholesterol, HDL-C and LDL-C levels, and increases ketone body formation.

6. The pharmaceutical composition according to claim 1, wherein the MPK38 enhances testosterone production.

7. The pharmaceutical composition according to claim 1, wherein the metabolic disease is selected from the group consisting of diabetes, hyperlipidemia, arteriosclerosis, high blood pressure, cardiovascular diseases, fatty liver, obesity-derived inflammatory diseases, obesity-derived autoimmune diseases, and obesity-derived cancer.

8. A method for preventing or treating obesity or a metabolic disease specific to middle-aged men, the method comprising administering to a subject in need thereof a pharmaceutical composition comprising a murine protein serine-threonine kinase 38 (MPK38) protein or a gene encoding the same as an active ingredient.

9. The method of claim 8, wherein the protein has an amino acid sequence of SEQ ID NO: 1.

10. The method of claim 8, wherein the gene has a nucleotide sequence of SEQ ID NO: 2.

11. The method of claim 8, wherein the MPK38 gene having the nucleotide sequence of SEQ ID NO: 2 is inserted into an expression vector.

12. The method of claim 8, wherein the MPK38 gene is specific to middle-aged men and reduces a size of adipocytes, inhibits expression of C/EBPα (CCAAT-enhancer-binding protein α) and PPARγ (peroxisome proliferator-activated receptor gamma and FABP4) as adipogenic genes, improves insulin sensitivity, reduces blood glucose and insulin, reduces blood triglyceride, total cholesterol, HDL-C and LDL-C levels, and increases ketone body formation.

13. The method of claim 8, wherein the MPK38 enhances testosterone production.

14. The method of claim 8, wherein the metabolic disease is selected from the group consisting of diabetes, hyperlipidemia, arteriosclerosis, high blood pressure, cardiovascular diseases, fatty liver, obesity-derived inflammatory diseases, obesity-derived autoimmune diseases, and obesity-derived cancer.
